(19) United States
(12) Patent Application Publication
(10) Pub. No.: US 2025/0214990 A1
(43) Pub. Date: Jul. 3, 2025

(54) DIACYLGLYCEROL KINASE (DGK) ALPHA INHIBITORS AND USES THEREOF

(71) Applicant: Insilico Medicine IP Limited, Hong Kong (HK)

(72) Inventors: Hongfu LU, Shanghai (CN); Huaxing YU, Shanghai (CN); Xiao DING, Shanghai (CN); Feng REN, Shanghai (CN)

(21) Appl. No.: 18/841,639

(22) PCT Filed: Mar. 1, 2023
(86) PCT No.: PCT/CN2023/078973

§ 371 Date: Aug. 26, 2024

(30) Foreign Application Priority Data
Mar. 1, 2022 (WO) PCT/CN2022/078655
May 6, 2022 (WO) PCT/CN2022/091215
Oct. 28, 2022 (WO) PCT/CN2022/128282
Jan. 12, 2023 (WO) PCT/CN2023/071962

(51) Int. Cl. C07D 471/04; A61K 31/5513; A61K 31/553; C07D 403/04; C07D 413/04; C07D 413/14; C07D 487/04; C07D 495/04; C07D 519/00

(52) U.S. Cl. CPC C07D 471/04; A61K 31/5513; A61K 31/553; C07D 403/04; C07D 413/04; C07D 413/14; C07D 487/04; C07D 495/04; C07D 519/00

(57) ABSTRACT

Described herein are DGKalpha inhibitors and pharmaceutical compositions comprising said inhibitors. The subject compounds and compositions are useful for the treatment of a disease or disorder associated with DGKalpha.

### CROSS-REFERENCE

This patent application claims the benefit of International Application No. PCT/CN2022/078655, filed Mar. 1, 2022; International Application No. PCT/CN2022/091215, filed May 6, 2022; International Application No. PCT/CN2022/128282, filed Oct. 28, 2022; and International Application No. PCT/CN2023/071962, filed Jan. 12, 2023; which are incorporated herein by reference in their entirety.

### BACKGROUND

Diacylglycerol kinases (DGKs) represent a family of enzymes that catalyze phosphorylation of the membrane lipid sn-1,2 diacylglycerol (DAG) to form phosphatidic acid (PA). In T cells, DAG is formed downstream of the T cell receptor (TCR) after activation of the gamma 1 isoform of phospholipase C (PLCyl) and cleavage of phosphatidylinositol 4,5-biphosphate (PIP2) into DAG and an additional second messenger, inositol 1,4,5-triphosphate (IP3). Whereas IP3 is important in facilitating release of calcium from the endoplasmic reticulum, DAG interacts with other proteins important in TCR signal transduction, such as Protein kinase CO and the Ras activating protein RasGRPI. Although, three isoforms of DGK are known to be present within T cells (DGKalpha, DGKdelta, and DGKzeta), only two, DGKalpha and DGKzeta, are thought to play an important role in facilitating DAG metabolism downstream of the TCR.

Supporting evidence include knock-out mouse models of either DGKalpha or DGKzeta which show a hyper-responsive T cell phenotype and improved anti-tumor immune activity (Riese M. J. et al., Journal of Biological Chemistry, (2011) 7: 5254-5265; Zha Y et al., Nature Immunology, (2006) 12:1343; Olenchock B. A. et al., (2006) 11: 1174-81). Furthermore tumor infiltrating lymphocytes isolated from human renal cell carcinoma patients were observed to overexpress DGKalpha which resulted in inhibited T cell function (Prinz, P. U. et al., J Immunology (2012) 12:5990-6000). Thus, DGKalpha and DGKzeta are viewed as targets for cancer immunotherapy (Riese M. J. et al., Front Cell Dev Biol. (2016) 4: 108; Chen, S. S. et al., Front Cell Dev Biol. (2016) 4: 130; Avila-Flores, A. et al., Immunology and Cell Biology (2017) 95: 549-563; Noessner, E., Front Cell Dev Biol. (2017) 5: 16; Krishna, S., et al., Front Immunology (2013) 4:178; Jing, W. et al., Cancer Research (2017) 77: 5676-5686. There remains a need for compounds useful as inhibitors of one or both of DGKalpha and DGKzeta, especially compounds that have selectivity over other diacylglycerol kinases, protein kinases, and/or other lipid kinases. There remains a need for compounds that are safe and effective in restoring T cell activation, lowering antigen threshold, enhancing antitumor functionality, and/or overcoming the suppressive effects of one or more endogenous immune checkpoints, such as PD-1, PD-L1, and CTLA-4, would be an important addition for the treatment of patients with proliferative disorders, such as cancer, as well as a viral infections.

### SUMMARY

Disclosed herein is a compound of Formula (A), or a pharmaceutically acceptable salt thereof:

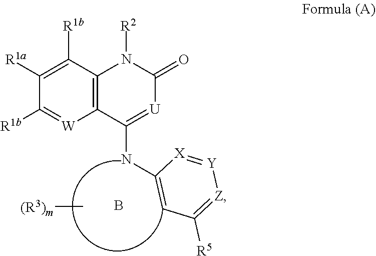

wherein:
R^{1a} is halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
W is N or CR^{1b};
each R^{1b} is independently hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R; R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
U is N or CR^{U};
R^{U} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Ring B is a heterocycloalkyl;
each R³ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R³ on the same atom are taken together to form an oxo;
or two R³ on the same or different carbons are taken together to form a cycloalkyl or heterocycloalkyl, each optionally substituted with one or more R;
m is 0-6;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
R⁵ is hydrogen, halogen, —CN, —NO₂, —OH, —OR^{a}, —OC(═O)R^{a}, —OC(═O)OR^{b}, —OC(═O)NR^{c}R^{d}, —SH, —SR, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl,

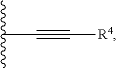

cycloalkyl, heterocycloalkyl, aryl, or heteroaryl; wherein each alkyl, alkenyl, cycloalkyl, heterocycloalkyl, aryl, and heteroaryl is optionally and independently substituted with one or more R;
R⁴ is hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{4a};
each R^{4a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R^{4a} on the same atom are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

In some embodiments of a compound of Formula (A), or a pharmaceutically acceptable salt thereof, the compound is of Formula (Aa):

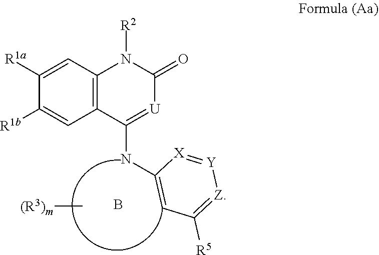

Also disclosed herein is a pharmaceutical composition comprising a compound disclosed herein, or a pharmaceutically acceptable salt thereof, and a pharmaceutically acceptable excipient.

Also disclosed herein is a method of inhibiting the activity of at least one of diacylglycerol kinase selected from diacylglycerol kinase alpha (DGKalpha) and diacylglycerol kinase zeta (DGKzeta), in a subject in need thereof, the method comprising administering to the subject a compound disclosed herein, or a pharmaceutically acceptable salt thereof.

Also disclosed herein is a method of inhibiting the activity of diacylglycerol kinase alpha (DGKalpha), in a subject in need thereof, the method comprising administering to the subject a compound disclosed herein, or a pharmaceutically acceptable salt thereof.

Also disclosed herein is a method of a disease associated with aberrant diacylglycerol kinase signaling, in a subject in need thereof, the method comprising administering to the subject a compound disclosed herein, or a pharmaceutically acceptable salt thereof.

In some embodiments, the diacylglycerol kinase is diacylglycerol kinase alpha. In some embodiments, the disease is cancer or a viral infection. In some embodiments, the method further comprises administering an additional therapeutic agent. In some embodiments, the additional therapeutic agent is an anti-cancer agent or an anti-viral agent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₆alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ fully saturated cycloalkyl or C₃-C₁₅ cycloalkenyl), from three to ten carbon atoms (C₃-C₁₀ fully saturated cycloalkyl or C₃-C₁₀ cycloalkenyl), from three to eight carbon atoms (C₃-C₈ fully saturated cycloalkyl or C₃-C₈ cycloalkenyl), from three to six carbon atoms (C₃-C₆ fully saturated cycloalkyl or C₃-C₆ cycloalkenyl), from three to five carbon atoms (C₃-C₅ fully saturated cycloalkyl or C₃-C₅ cycloalkenyl), or three to four carbon atoms (C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ fully saturated heterocycloalkyl or C₂-C₁₅ heterocycloalkenyl), from two to ten carbon atoms (C₂-C₁₀ fully saturated heterocycloalkyl or C₂-C₁₀ heterocycloalkenyl), from two to eight carbon atoms (C₂-C₈ fully saturated heterocycloalkyl or C₂-C₅ heterocycloalkenyl), from two to seven carbon atoms (C₂-C₇ fully saturated heterocycloalkyl or C₂-C₇ heterocycloalkenyl), from two to six carbon atoms (C₂-C₆ fully saturated heterocycloalkyl or C₂-C₆ heterocycloalkenyl), from two to five carbon atoms (C₂-C₅ fully saturated heterocycloalkyl or C₂-C₅ heterocycloalkenyl), or two to four carbon atoms (C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

As used herein, a “disease or disorder associated with DGK” or, alternatively, “a DGK-mediated disease or disorder” means any disease or other deleterious condition in which DGK, or a mutant thereof, is known or suspected to play a role.

As used herein, a “disease or disorder associated with DGKalpha” or, alternatively, “a DGKalpha-mediated disease or disorder” means any disease or other deleterious condition in which DGKalpha, or a mutant thereof, is known or suspected to play a role.

As used herein, a “disease or disorder associated with DGKzeta” or, alternatively, “a DGKzeta-mediated disease or disorder” means any disease or other deleterious condition in which DGKzeta, or a mutant thereof, is known or suspected to play a role.

### Compounds

Described herein are compounds, or a pharmaceutically acceptable salt thereof useful in the treatment of a disease or disorder associated with DGK, especially DGKalpha.

Disclosed herein is a compound of Formula (I), or a pharmaceutically acceptable salt thereof:

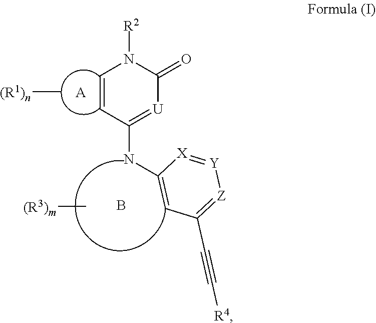

wherein:
Ring A is cycloalkyl, heterocycloalkyl, aryl, or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R¹ on the same atom are taken together to form an oxo;
n is 0-4;
R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
U is N or CR^{U};
R^{U} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Ring B is a heterocycloalkyl;
each R³ is independently halogen, —CN, —NO₂, —OH, —OR^{a}, —OC(═O)R^{a}, —OC(═O)OR^{b}, —OC(═O)NR^{c}R^{d}, —SH, —SR, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R³ on the same atom are taken together to form an oxo;
or two R³ on the same or different carbons are taken together to form a cycloalkyl or heterocycloalkyl, each optionally substituted with one or more R;
m is 0-6;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
R⁴ is hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{a};
each R^{4a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R^{4a} on the same atom are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

Disclosed herein is a compound of Formula (I), or a pharmaceutically acceptable salt thereof:

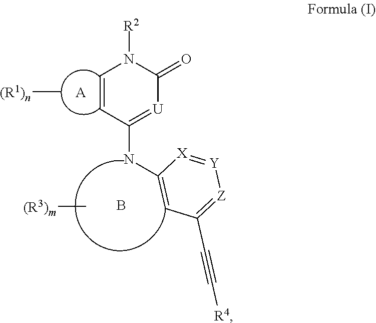

wherein:
Ring A is cycloalkyl, heterocycloalkyl, aryl, or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R¹ on the same atom are taken together to form an oxo;
n is 0-4;
R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
U is N or CR^{U};
R^{U} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Ring B is a heterocycloalkyl;
each R³ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R³ on the same atom are taken together to form an oxo;
or two R³ on the same or different carbons are taken together to form a cycloalkyl or heterocycloalkyl, each optionally substituted with one or more R;
m is 0-6;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Z is N or CR^{Z};
R^{Z} is hydrogen, halogen, —CN, —OH, —OR, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
R⁴ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{4a};
each R^{4a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R^{4a} on the same atom are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

Disclosed herein is a compound of Formula (II), or a pharmaceutically acceptable salt thereof:

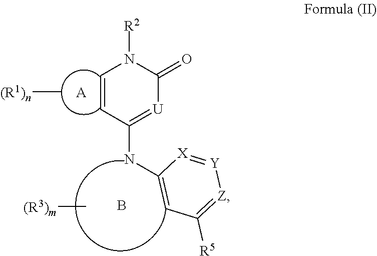

wherein:
Ring A is cycloalkyl, heterocycloalkyl, aryl, or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R¹ on the same atom are taken together to form an oxo;
n is 0-4;
R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
U is N or CR^{U};
R^{U} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Ring B is a heterocycloalkyl;
each R³ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R³ on the same atom are taken together to form an oxo;
or two R³ on the same or different carbons are taken together to form a cycloalkyl or heterocycloalkyl, each optionally substituted with one or more R;
m is 0-6;
X is N or CR^{X};
R^{X} is hydrogen, halogen, —CN, —OH, —OR^{a}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
R⁵ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl,

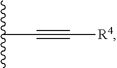

cycloalkyl, heterocycloalkyl, aryl, or heteroaryl; wherein each alkyl, alkenyl, cycloalkyl, heterocycloalkyl, aryl, and heteroaryl is optionally and independently substituted with one or more R;
R⁴ is hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R;
each R^{4a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R^{4a} on the same atom are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

In some embodiments, a compound of Formula (II) has a structure of Formula (I).

In some embodiments of a compound of Formula (II), R⁵ is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl,

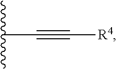

cycloalkyl, heterocycloalkyl, aryl, or heteroaryl; wherein each alkyl, cycloalkyl, heterocycloalkyl, aryl, and heteroaryl is optionally and independently substituted with one or more R.

In some embodiments of a compound of Formula (II), R⁵ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl,

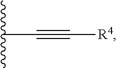

cycloalkyl, heterocycloalkyl, aryl, or heteroaryl; wherein each alkyl, cycloalkyl, heterocycloalkyl, aryl, and heteroaryl is optionally and independently substituted with one or more R.

In some embodiments of a compound of Formula (II), R⁵ is hydrogen, halogen, —OR, —NR^{c}R^{d}, C₁-C₆alkyl, C₁-C₆haloalkyl,

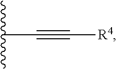

cycloalkyl, or heterocycloalkyl; wherein each alkyl, cycloalkyl, and heterocycloalkyl is optionally and independently substituted with one or more R.

In some embodiments of a compound of Formula (II), R⁵ is hydrogen, —OR^{a}, C₁-C₆alkyl,

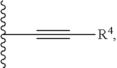

cycloalkyl, or heterocycloalkyl; wherein each alkyl, cycloalkyl, and heterocycloalkyl is optionally and independently substituted with one or more R.

In some embodiments of a compound of Formula (II), R⁵ is hydrogen.

In some embodiments of a compound of Formula (II), R⁵ is —OR^{a}. In some embodiments of a compound of Formula (II), R⁵ is —O—C₁-C₆alkyl, wherein the alkyl is optionally substituted. In some embodiments of a compound of Formula (II), R⁵ is —O—C₁-C₆halolkyl. In some embodiments of a compound of Formula (II), R⁵ is —O-cycloalkyl. In some embodiments of a compound of Formula (II), R⁵ is —O-cycloalkyl, wherein the cycloalkyl is 3-6 membered ring and is optionally substituted with one or more R. In some embodiments of a compound of Formula (II), R⁵ is —O-heterocycloalkyl, wherein the heterocycloalkyl is 5-6 membered ring and is optionally substituted with one or more R.

In some embodiments of a compound of Formula (II), R⁵ is C₁-C₆alkyl.

In some embodiments of a compound of Formula (II), R⁵ is

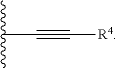

In some embodiments of a compound of Formula (II), R⁵ is cycloalkyl or heterocycloalkyl; wherein each cycloalkyl and heterocycloalkyl is optionally and independently substituted with one or more R.

In some embodiments of a compound of Formula (I) or (II), Ring A is aryl or heteroaryl. In some embodiments of a compound of Formula (I) or (II), Ring A is phenyl. In some embodiments of a compound of Formula (I) or (II), Ring A is 6-membered heteroaryl. In some embodiments of a compound of Formula (I) or (II), Ring A is pyridinyl. In some embodiments of a compound of Formula (I) or (II), Ring A is 5-membered heteroaryl. In some embodiments of a compound of Formula (I) or (II), Ring A is thiophenyl, furanyl, pyrrolyl, imidazolyl, pyrazolyl, thiazolyl, oxazolyl, or triazolyl. In some embodiments of a compound of Formula (I) or (II), Ring A is thiophenyl, furanyl, or pyrrolyl. In some embodiments of a compound of Formula (I) or (II), Ring A is thiophenyl.

In some embodiments of a compound of Formula (I) or (II), the compound if of Formula (Ia):

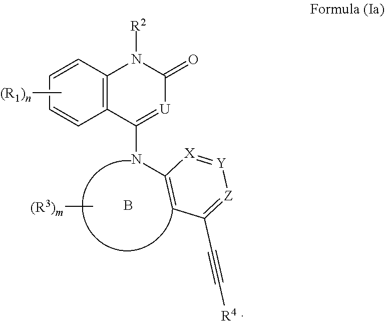

In some embodiments of a compound of Formula (I) or (II), the compound if of Formula (Ib):

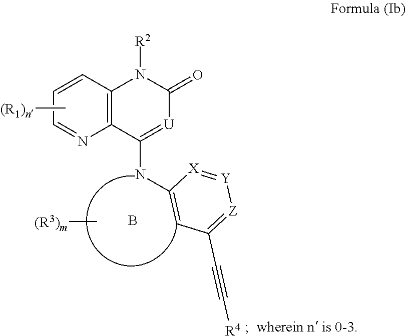

In some embodiments of a compound of Formula (I) or (II), the compound if of Formula (Ic):

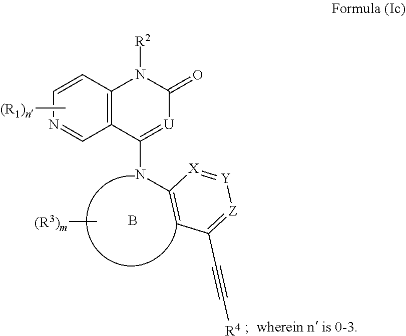

In some embodiments of a compound of Formula (I) or (II), the compound if of Formula (Id):

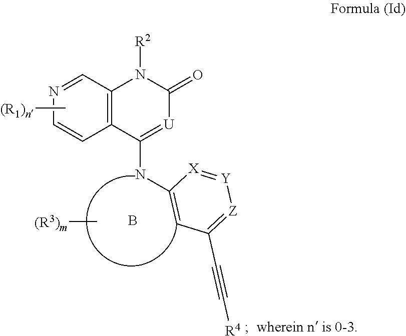

In some embodiments of a compound of Formula (I) or (II), the compound if of Formula (Ie):

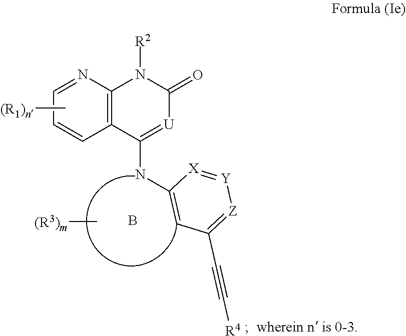

In some embodiments of a compound of Formula (I) or (II), the compound if of Formula (If):

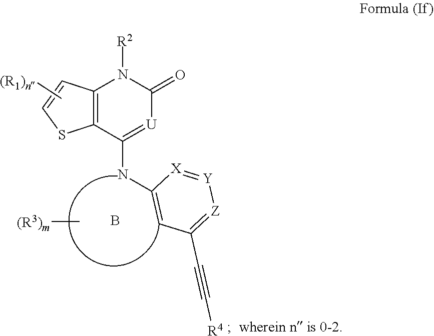

In some embodiments of a compound of Formula (I) or (II), the compound if of Formula (Ig):

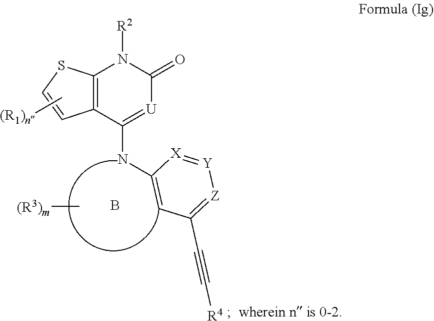

In some embodiments of a compound of Formula (I), (Ia)-(Ig), or (II), each R¹ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (I), (Ia)-(Ig), or (IT), each R¹ is independently halogen, —CN, —OH, —OR^{a}, —NR^{c}R^{d}, cycloalkyl, or heterocycloalkyl. In some embodiments of a compound of Formula (I), (la)-(Ig), or (II), each R¹ is independently halogen, —CN, —OR^{a}, —NR^{c}R^{d}, or heterocycloalkyl. In some embodiments, the heterocycloalkyl is 5- or 6-membered heterocycloalkyl. In some embodiments, the heterocycloalkyl is 6-membered heterocycloalkyl. In some embodiments, the heterocycloalkyl comprises 0-2 nitrogen atoms and 0-1 oxygen atom. In some embodiments, the heterocycloalkyl comprises 1 or 2 nitrogen atoms. In some embodiments, the heterocycloalkyl comprises 1 oxygen atom. In some embodiments, the heterocycloalkyl comprises 1 nitrogen atom and 1 oxygen atom. In some embodiments of a compound of Formula (I), (Ia)-(Ig), or (II), each R¹ is independently halogen, —CN, —OR^{a}, or —NR^{c}R^{d}. In some embodiments of a compound of Formula (I), (Ia)-(Ig), or (II), each R¹ is independently halogen, —CN, —OH, —OR^{a}, —NR^{c}R^{d}, C₁-C₆alkyl, or C₁-C₆haloalkyl. In some embodiments of a compound of Formula (I), (Ia)-(Ig), or (II), each R¹ is independently halogen, —CN, C₁-C₆alkyl, or C₁-C₆haloalkyl. In some embodiments of a compound of Formula (I), (Ia)-(Ig), or (II), each R¹ is independently halogen or —CN. In some embodiments of a compound of Formula (I), (Ia)-(Ig), or (II), each R¹ is independently halogen. In some embodiments of a compound of Formula (I), (Ia)-(Ig), or (II), each R¹ is independently fluoro or chloro. In some embodiments of a compound of Formula (I), (Ia)-(Ig), or (II), each R¹ is —CN. In some embodiments of a compound of Formula (I), (Ia)-(Ig), or (II), —OR is —O-heterocycloalkyl, —O—C₁-C₆alkyl, —O—C₁-C₆haloalkyl, —O—C₁-C₆hydroxylalkyl, —O—C₁-C₆aminoalkyl. In some embodiments of a compound of Formula (I) or (Ia)-(Ig) Formula (I), (Ia)-(Ig), or (II), —OR^{a} is —O-heterocycloalkyl, wherein the heterocycloalkyl is a 5 or 6 membered ring. In some embodiments of a compound of Formula (I), (Ia)-(Ig), or (II), —OR^{a} is

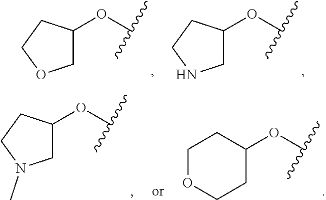

In some embodiments of a compound of Formula (I), (Ia)-(Ig), or (II), —OR^{a} is —O—C₁-C₆alkyl. In some embodiments of a compound of Formula (I), (Ia)-(Ig), or (II), —OR^{a} is —O—C₁-C₆haloalkyl. In some embodiments of a compound of Formula (I), (Ia)-(Ig), or (II), —OR^{a} is —O—C₁-C₆hydroxylalkyl. In some embodiments of a compound of Formula (I), (Ia)-(Ig), or (II), —OR^{a} is —O—C₁-C₆aminoalkyl.

In some embodiments of a compound of Formula (I), (Ia) or (II), n is 0-3. In some embodiments of a compound of Formula (I), (Ia) or (II), n is 0-2. In some embodiments of a compound of Formula (I), (Ia) or (II), n is 0 or 1. In some embodiments of a compound of Formula (I), (Ia) or (II), n is 0. In some embodiments of a compound of Formula (I), (Ia) or (II), n is 1. In some embodiments of a compound of Formula (I), (Ia) or (II), n is 2.

In some embodiments of a compound of Formula (Ib)-(Ie), n′ is 0-2. In some embodiments of a compound of Formula (Ib)-(Ie), n′ is 0 or 1. In some embodiments of a compound of Formula (Ib)-(Ie), n′ is 0. In some embodiments of a compound of Formula (Ib)-(Ie), n′ is 1. In some embodiments of a compound of Formula (Ib)-(Ie), n′ is 2.

In some embodiments of a compound of Formula (If) or (Ig), n″ is 0 or 1. In some embodiments of a compound of Formula (If) or (Ig), n″ is 0. In some embodiments of a compound of Formula (If) or (Ig), n″ is 1. In some embodiments of a compound of Formula (If) or (Ig), n″ is 2.

Disclosed herein is a compound of Formula (A), or a pharmaceutically acceptable salt thereof:

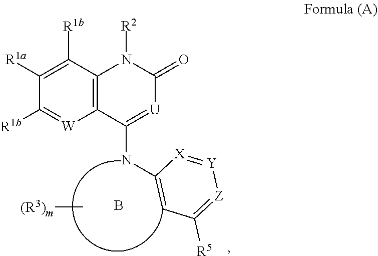

wherein:
R^{1a} is halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
W is N or CR^{1b};
each R^{1b} is independently hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
U is N or CR^{U};
R^{U} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Ring B is a heterocycloalkyl;
each R³ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R³ on the same atom are taken together to form an oxo;
or two R³ on the same or different carbons are taken together to form a cycloalkyl or heterocycloalkyl, each optionally substituted with one or more R;
m is 0-6;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
R⁵ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl,

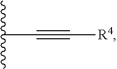

cycloalkyl, heterocycloalkyl, aryl, or heteroaryl; wherein each alkyl, alkenyl, cycloalkyl, heterocycloalkyl, aryl, and heteroaryl is optionally and independently substituted with one or more R;
R⁴ is hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{a};
each R^{4a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R^{4a} on the same atom are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

In some embodiments of a compound of Formula (A), R^{1a} is halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (A), R^{1a} is halogen, —CN, —OH, —OR^{a}, —NR^{c}R^{d}, C₁-C₆alkyl, or C₁-C₆haloalkyl. In some embodiments of a compound of Formula (A), R^{1a} is halogen, —CN, C₁-C₆alkyl, or C₁-C₆haloalkyl. In some embodiments of a compound of Formula (A), R^{1a} is independently halogen, —CN, —OR^{a}, or —NR^{C}R^{d}. In some embodiments of a compound of Formula (A), R^{1a} is —CN or —OR^{a}. In some embodiments of a compound of Formula (A), R^{1a} is —CN, —O-heterocycloalkyl, —O—C₁-C₆alkyl, —O—C₁-C₆haloalkyl, —O—C₁-C₆hydroxylalkyl, or —O—C₁-C₆aminoalkyl. In some embodiments of a compound of Formula (A), R^{1a} is —CN, —O-heterocycloalkyl, —O—C₁-C₃alkyl, —O—C₁-C₃haloalkyl, —O—C₁-C₃hydroxylalkyl, or —O—C₁-C₃aminoalkyl. In some embodiments of a compound of Formula (A), R^{1a} is independently —CN or —NR^{c}R^{d}. In some embodiments of a compound of Formula (A), —OR^{a} is —O-heterocycloalkyl, —O—C₁-C₆alkyl, —O—C₁-C₆haloalkyl, —O—C₁-C₆hydroxylalkyl, or —O—C₁-C₆aminoalkyl. In some embodiments of a compound of Formula (A), —OR^{a} is —O-heterocycloalkyl, wherein the heterocycloalkyl is a 5 or 6 membered ring. In some embodiments of a compound of Formula (A), —OR^{a} is

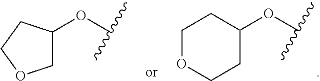 [SMILES: *OC1CCOC1 ; *OC1CCOCC1]

In some embodiments of a compound of Formula (Aa), —OR^{a} is —O—C₁-C₆alkyl. In some embodiments of a compound of Formula (A), —OR^{a} is —O—C₁-C₆haloalkyl. In some embodiments of a compound of Formula (A), —OR^{a} is —O—C₁-C₆hydroxylalkyl. In some embodiments of a compound of Formula (A), —OR^{a} is —O—C₁-C₃hydroxylalkyl. In some embodiments of a compound of Formula (A), —OR^{a} is —O—CH₂CH₂OH. In some embodiments of a compound of Formula (A), —OR^{a} is —O—C₁-C₆aminoalkyl.

In some embodiments of a compound of Formula (A), R^{1a} is —OR^{a}, —NR^{c}R^{d}, or heterocycloalkyl. In some embodiments of a compound of Formula (A), R^{1a} is —OR^{a}. In some embodiments of a compound of Formula (A), R^{1a} is —O-heterocycloalkyl, —O—C₁-C₆hydroxylalkyl, —NH—C₁-C₆hydroxylalkyl, or —O—C₁-C₆aminoalkyl. In some embodiments of a compound of Formula (A), Ria is —O-heterocycloalkyl, —O—C₁-C₆hydroxylalkyl, or —O—C₁-C₆aminoalkyl. In some embodiments of a compound of Formula (A), R^{1a} is —O-heterocycloalkyl. In some embodiments of a compound of Formula (A), R^{1a} is —O-5-6 membered heterocycloalkyl. In some embodiments of a compound of Formula (A), R^{1a} is —O—C₁-C₆hydroxylalkyl. In some embodiments of a compound of Formula (A), R^{1a} is —O—C₁-C₃hydroxylalkyl. In some embodiments of a compound of Formula (A), R^{1a} is —O—CH₂CH₂OH. In some embodiments of a compound of Formula (A), R^{1a} is —O—C₁-C₆aminoalkyl.

In some embodiments of a compound of Formula (A), W is N. In some embodiments of a compound of Formula (A), W is CR^{1b}.

In some embodiments of a compound of Formula (A), each R^{1b} is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (A), each R^{1b} is independently hydrogen, halogen, —CN, C₁-C₆alkyl, C₁-C₆haloalkyl, C₁-C₆hydroxyalkyl, or C₁-C₆aminoalkyl. In some embodiments of a compound of Formula (A), each R^{1b} is independently hydrogen, halogen, or —CN. In some embodiments of a compound of Formula (A), each R^{1b} is independently hydrogen or halogen. In some embodiments of a compound of Formula (A), each R^{1b} is independently hydrogen or —CN.

In some embodiments of a compound of Formula (A), or a pharmaceutically acceptable salt thereof, the compound is of Formula (Aa):

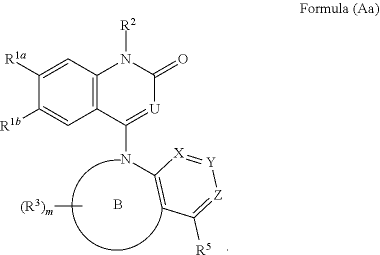

In some embodiments of a compound of Formula (A), or a pharmaceutically acceptable salt thereof, the compound is of Formula (Ab):

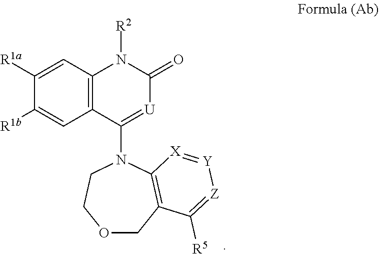

In some embodiments of a compound of Formula (A), or a pharmaceutically acceptable salt thereof, the compound is of Formula (Ac):

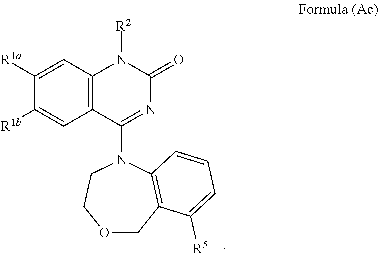

In some embodiments of a compound of Formula (Aa), (Ab), or (Ac), R^{1a} is halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (Aa), (Ab), or (Ac), R^{1a} is halogen, —CN, —OH, —OR^{a}, —NR^{c}R^{d}, C₁-C₆alkyl, or C₁-C₆haloalkyl. In some embodiments of a compound of Formula (Aa), (Ab), or (Ac), R^{1a} is halogen, —CN, C₁-C₆alkyl, or C₁-C₆haloalkyl. In some embodiments of a compound of Formula (Aa), (Ab), or (Ac), R^{1a} is independently halogen, —CN, —OR^{a}, or —NR^{c}R^{d}. In some embodiments of a compound of Formula (Aa), (Ab), or (Ac), R^{1a} is —CN or —OR^{a}. In some embodiments of a compound of Formula (Aa), (Ab), or (Ac), R^{1a} is —CN, —O-heterocycloalkyl, —O—C₁-C₆alkyl, —O—C₁-C₆haloalkyl, —O—C₁-C₆hydroxylalkyl, or —O—C₁-C₆aminoalkyl. In some embodiments of a compound of Formula (Aa), (Ab), or (Ac), R^{a} is —CN, —O-heterocycloalkyl, —O—C₁-C₃alkyl, —O—C₁-C₃haloalkyl, —O—C₁-C₃hydroxylalkyl, or —O—C₁-C₃aminoalkyl. In some embodiments of a compound of Formula (Aa), (Ab), or (Ac), R^{1a} is independently —CN or —NR^{c}R^{d}. In some embodiments of a compound of Formula (Aa), (Ab), or (Ac), —OR^{a} is —O-heterocycloalkyl, —O—C₁-C₆alkyl, —O—C₁-C₆haloalkyl, —O—C₁-C₆hydroxylalkyl, or —O—C₁-C₆aminoalkyl. In some embodiments of a compound of Formula (Aa), (Ab), or (Ac), —OR^{a} is —O-heterocycloalkyl, wherein the heterocycloalkyl is a 5 or 6 membered ring. In some embodiments of a compound of Formula (Aa), (Ab), or (Ac), —OR^{a} is

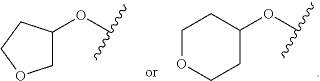 [SMILES: *OC1CCOC1 ; *OC1CCOCC1]

In some embodiments of a compound of Formula (Aa), (Ab), or (Ac), —OR^{a} is —O—C₁-C₆alkyl. In some embodiments of a compound of Formula (Aa), (Ab), or (Ac), —OR^{a} is —O—C₁-C₆haloalkyl. In some embodiments of a compound of Formula (Aa), (Ab), or (Ac), —OR^{a} is —O—C₁-C₆hydroxylalkyl. In some embodiments of a compound of Formula (Aa), (Ab), or (Ac), —OR^{a} is —O—C₁-C₆aminoalkyl.

In some embodiments of a compound of Formula (Aa), (Ab), or (Ac), R^{1a} is —OR^{a}, —NR^{c}R^{d}, or heterocycloalkyl. In some embodiments of a compound of Formula (Aa), (Ab), or (Ac), R^{1a} is —OR^{a}. In some embodiments of a compound of Formula (Aa), (Ab), or (Ac), R^{1a} is —O-heterocycloalkyl, —O—C₁-C₆hydroxylalkyl, —NH—C₁-C₆hydroxylalkyl, or —O—C₁-C₆aminoalkyl. In some embodiments of a compound of Formula (Aa), (Ab), or (Ac), R^{1a} is —O-heterocycloalkyl, —O—C₁-C₆hydroxylalkyl, or —O—C₁-C₆aminoalkyl. In some embodiments of a compound of Formula (Aa), (Ab), or (Ac), R^{1a} is —O— heterocycloalkyl. In some embodiments of a compound of Formula (Aa), (Ab), or (Ac), R^{1a} is —O—C₁-C₆hydroxylalkyl. In some embodiments of a compound of Formula (Aa), (Ab), or (Ac), R^{1a} is —O—C₁-C₃hydroxylalkyl. In some embodiments of a compound of Formula (Aa), (Ab), or (Ac), R^{1a} is —O—CH₂CH₂OH. In some embodiments of a compound of Formula (Aa), (Ab), or (Ac), R^{1a} is —O—C₁-C₆aminoalkyl.

In some embodiments of a compound of Formula (Aa), (Ab), or (Ac), R^{1b} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (Aa), (Ab), or (Ac), R^{1b} is hydrogen, halogen, —CN, C₁-C₆alkyl, C₁-C₆haloalkyl, C₁-C₆hydroxyalkyl, or C₁-C₆aminoalkyl. In some embodiments of a compound of Formula (Aa), (Ab), or (Ac), R^{1b} is halogen, —CN, C₁-C₆alkyl, C₁-C₆haloalkyl, C₁-C₆hydroxyalkyl, or C₁-C₆aminoalkyl. In some embodiments of a compound of Formula (Aa), (Ab), or (Ac), R^{1b} is hydrogen, halogen, or —CN. In some embodiments of a compound of Formula (Aa), (Ab), or (Ac), R^{1b} is halogen or —CN. In some embodiments of a compound of Formula (Aa), (Ab), or (Ac), R^{1b} is hydrogen or —CN. In some embodiments of a compound of Formula (Aa), (Ab), or (Ac), R^{1b} is —CN. In some embodiments of a compound of Formula (Aa), (Ab), or (Ac), R^{1b} is hydrogen or halogen. In some embodiments of a compound of Formula (Aa), (Ab), or (Ac), R^{1b} is halogen. In some embodiments of a compound of Formula (Aa), (Ab), or (Ac), R^{1b} is Cl.

In some embodiments of a compound of Formula (Aa), (Ab), or (Ac), R^{1b} is halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (Aa), (Ab), or (Ac), R^{1b} is halogen, —CN, —OH, —OR^{a}, —NR^{c}R^{d}, C₁-C₆alkyl, or C₁-C₆haloalkyl. In some embodiments of a compound of Formula (Aa), (Ab), or (Ac), R^{1b} is halogen, —CN, C₁-C₆alkyl, or C₁-C₆haloalkyl. In some embodiments of a compound of Formula (Aa), R^{1b} is halogen or —CN. In some embodiments of a compound of Formula (Aa), (Ab), or (Ac), R^{1b} is halogen. In some embodiments of a compound of Formula (Aa), (Ab), or (Ac), R^{1b} is fluoro or chloro. In some embodiments of a compound of Formula (Aa), (Ab), or (Ac), R^{1b} is —CN. In some embodiments of a compound of Formula (Aa), (Ab), or (Ac), R^{Ib} is independently halogen, —CN, —OR^{a}, or —NR^{c}R^{d}. In some embodiments of a compound of Formula (Aa), (Ab), or (Ac), R^{1b} is —CN or —OR^{a}. In some embodiments of a compound of Formula (Aa), (Ab), or (Ac), R^{1b} is —CN, —O-heterocycloalkyl, —O—C₁-C₆alkyl, —O—C₁-C₆haloalkyl, —O—C₁-C₆hydroxylalkyl, or —O—C₁-C₆aminoalkyl. In some embodiments of a compound of Formula (Aa), (Ab), or (Ac), R^{Ib} is —CN, —O-heterocycloalkyl, —O—C₁-C₃alkyl, —O—C₁-C₃haloalkyl, —O—C₁-C₃hydroxylalkyl, or —O—C₁-C₃aminoalkyl. In some embodiments of a compound of Formula (Aa), (Ab), or (Ac), R^{1b} is independently —CN or —NR^{c}R^{d}. In some embodiments of a compound of Formula (Aa), (Ab), or (Ac), —OR^{a} is —O-heterocycloalkyl, —O—C₁-C₆alkyl, —O—C₁-C₆haloalkyl, —O—C₁-C₆hydroxylalkyl, or —O—C₁-C₆aminoalkyl. In some embodiments of a compound of Formula (Aa), (Ab), or (Ac), —OR^{a} is —O-heterocycloalkyl, wherein the heterocycloalkyl is a 5 or 6 membered ring. In some embodiments of a compound of Formula (Aa), (Ab), or (Ac), —OR^{a} is

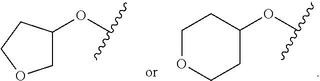 [SMILES: *OC1CCOC1 ; *OC1CCOCC1]

In some embodiments of a compound of Formula (Aa), (Ab), or (Ac), —OR^{a} is —O—C₁-C₆alkyl. In some embodiments of a compound of Formula (Aa), (Ab), or (Ac), —OR^{a} is —O—C₁-C₆haloalkyl. In some embodiments of a compound of Formula (Aa), (Ab), or (Ac), —OR^{a} is —O—C₁-C₆hydroxylalkyl. In some embodiments of a compound of Formula (Aa), (Ab), or (Ac), —OR^{a} is —O—C₁-C₆aminoalkyl.

In some embodiments of a compound of Formula (Aa), (Ab), or (Ac), R^{1a} is —O—C₁-C₆hydroxylalkyl and R^{1b} is CN. In some embodiments of a compound of Formula (Aa), (Ab), or (Ac), R^{1a} is —O—C₁-C₃hydroxylalkyl and R^{1b} is CN. In some embodiments of a compound of Formula (Aa), (Ab), or (Ac), R^{1a} is —O—CH₂CH₂OH and R^{1b} is CN.

In some embodiments of a compound of Formula (Aa), (Ab), or (Ac), R^{1a} is —O—C₁-C₆hydroxylalkyl and R^{1b} is halogen. In some embodiments of a compound of Formula (Aa), (Ab), or (Ac), R^{1a} is —O—C₁-C₃hydroxylalkyl and R^{1b} is halogen. In some embodiments of a compound of Formula (Aa), (Ab), or (Ac), R^{1a} is —O—CH₂CH₂OH and R^{1b} is halogen.

In some embodiments of a compound of Formula (Aa), (Ab), or (Ac), R^{1a} is —O—C₁-C₆hydroxylalkyl and R^{1b} is chloro. In some embodiments of a compound of Formula (Aa), (Ab), or (Ac), R^{1a} is —O—C₁-C₃hydroxylalkyl and R^{1b} is chloro. In some embodiments of a compound of Formula (Aa), (Ab), or (Ac), R^{1a} is —O—CH₂CH₂OH and R^{1b} is chloro.

In some embodiments of a compound of Formula (A), (Aa), (Ab), or (Ac), R⁵ is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl,

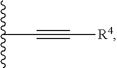

cycloalkyl, heterocycloalkyl, aryl, or heteroaryl; wherein each alkyl, cycloalkyl, heterocycloalkyl, aryl, and heteroaryl is optionally and independently substituted with one or more R.

In some embodiments of a compound of Formula (A), (Aa), (Ab), or (Ac), R⁵ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl,

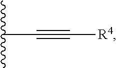

cycloalkyl, heterocycloalkyl, aryl, or heteroaryl; wherein each alkyl, cycloalkyl, heterocycloalkyl, aryl, and heteroaryl is optionally and independently substituted with one or more R.

In some embodiments of a compound of Formula (A), (Aa), (Ab), or (Ac), R⁵ is hydrogen, halogen, —OR^{a}, —NR^{c}R^{d}, C₁-C₆alkyl, C₁-C₆haloalkyl,

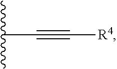

cycloalkyl, or heterocycloalkyl; wherein each alkyl, cycloalkyl, and heterocycloalkyl is optionally and independently substituted with one or more R.

In some embodiments of a compound of Formula (A), (Aa), (Ab), or (Ac), R⁵ is hydrogen, —OR^{a}, C₁-C₆alkyl,

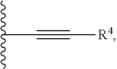

3-6 membered cycloalkyl, or 5-6 membered heterocycloalkyl; wherein each alkyl, cycloalkyl, and heterocycloalkyl is optionally and independently substituted with one or more R.

In some embodiments of a compound of Formula (A), (Aa), (Ab), or (Ac), R⁵ is hydrogen.

In some embodiments of a compound of Formula (A), (Aa), (Ab), or (Ac), R⁵ is —OR. In some embodiments of a compound of Formula (A), (Aa), (Ab), or (Ac), R⁵ is —O—C₁-C₆alkyl, wherein the alkyl is optionally substituted. In some embodiments of a compound of Formula (A), (Aa), (Ab), or (Ac), R⁵ is —O—C₁-C₆halolkyl. In some embodiments of a compound of Formula (A), (Aa), (Ab), or (Ac), R⁵ is —O— cycloalkyl. In some embodiments of a compound of Formula (A), (Aa), (Ab), or (Ac), R⁵ is —O— cycloalkyl, wherein the cycloalkyl is 3-6 membered ring and is optionally substituted with one or more R. In some embodiments of a compound of Formula (A), (Aa), (Ab), or (Ac), R⁵ is —O-heterocycloalkyl, wherein the heterocycloalkyl is 5-6 membered ring and is optionally substituted with one or more R.

In some embodiments of a compound of Formula (A), (Aa), (Ab), or (Ac), R⁵ is C₁-C₆alkyl.

In some embodiments of a compound of Formula (A), (Aa), (Ab), or (Ac), R⁵ is

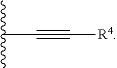

In some embodiments of a compound of Formula (A), (Aa), (Ab), or (Ac), R⁵ is 3-6 membered cycloalkyl or 5-6 membered heterocycloalkyl; wherein each cycloalkyl and heterocycloalkyl is optionally and independently substituted with one or more R.

In some embodiments of a compound of Formula (A), (Aa), (Ab), (Ac) (I), (Ia)-(Ig), or (II), R² is hydrogen or C₁-C₆alkyl. In some embodiments of a compound of Formula (A), (Aa), (Ab), (Ac) (I), (Ia)-(Ig), or (II), R² is C₁-C₆alkyl. In some embodiments of a compound of Formula (A), (Aa), (Ab), (Ac) (I), (Ia)-(Ig), or (II), R² is C₁-C₃alkyl. In some embodiments of a compound of Formula (A), (Aa), (Ab), (Ac) (I), (Ia)-(Ig), or (II), R² is methyl.

In some embodiments of a compound of Formula (A), (Aa), (Ab), (I), (Ia)-(Ig), or (II), U is N. In some embodiments of a compound of Formula (A), (Aa), (Ab), (I), (Ia)-(Ig), or (II), U is CR^{U}.

In some embodiments of a compound of Formula (A), (Aa), (Ab), (I), (Ia)-(Ig), or (II), R^{U} is hydrogen, halogen, —CN, —OH, —OR^{a}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (A), (Aa), (Ab), (I), (Ia)-(Ig), or (II), R^{U} is hydrogen, halogen, —CN, —C(═O)NR^{c}R^{d}, C₁-C₆alkyl, or C₁-C₆haloalkyl. In some embodiments of a compound of Formula (A), (Aa), (Ab), (I), (Ia)-(Ig), or (II), R^{U} is hydrogen, —CN, or —C(═O)NR^{c}R^{d}. In some embodiments of a compound of Formula (A), (Aa), (Ab), (I), (Ia)-(Ig), or (II), R^{U} is hydrogen or —CN. In some embodiments of a compound of Formula (A), (Aa), (Ab), (I), (la)-(Ig), or (II), R^{U} is —CN. In some embodiments of a compound of Formula (A), (Aa), (Ab), (I), (Ia)-(Ig), or (II), R^{U} is hydrogen.

In some embodiments of a compound of Formula (I), (II), (A), (Aa), or (Ab),

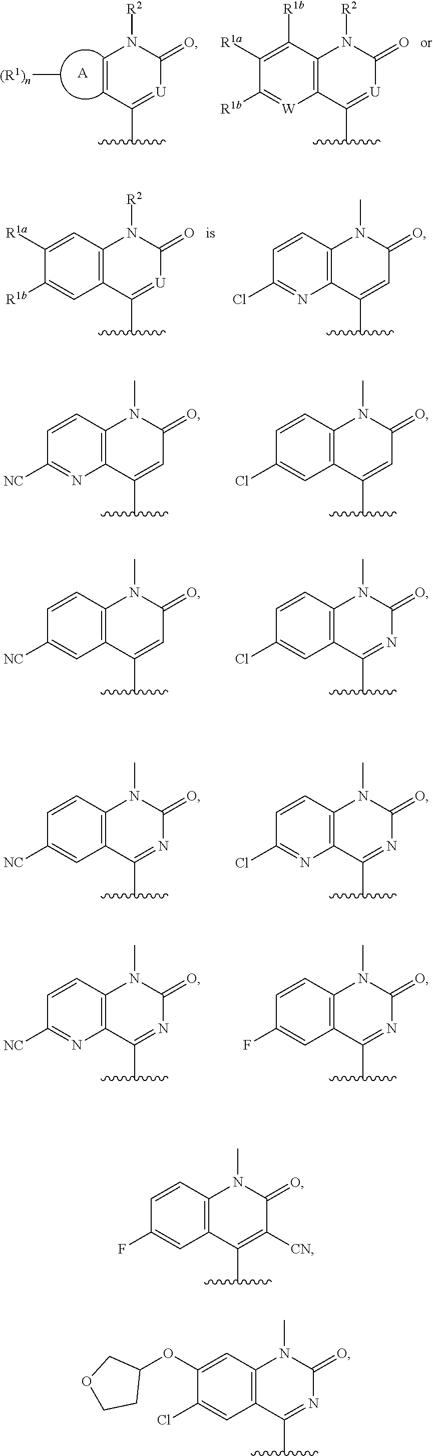

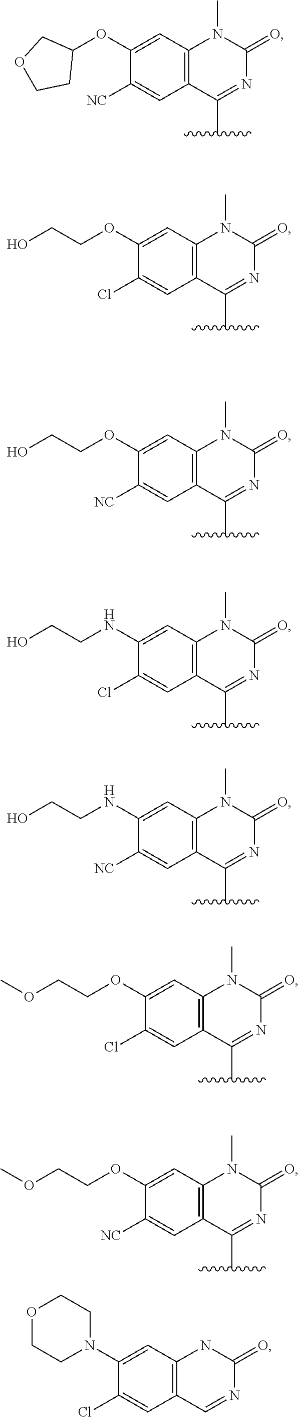

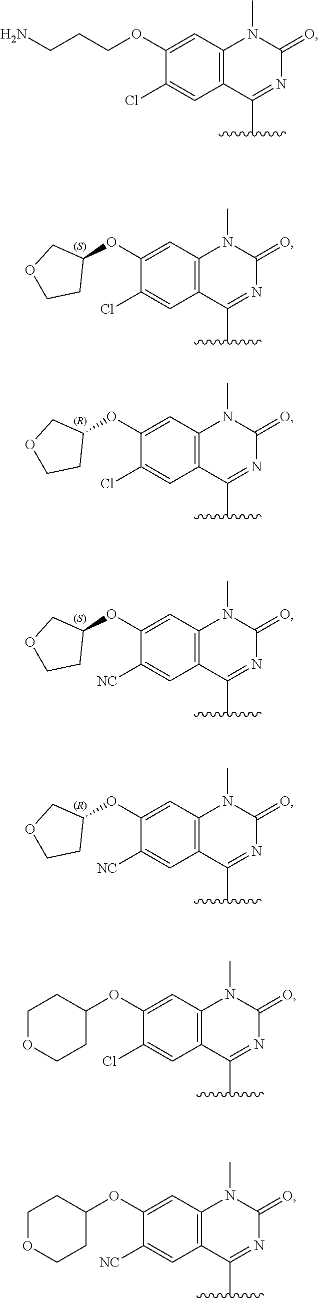

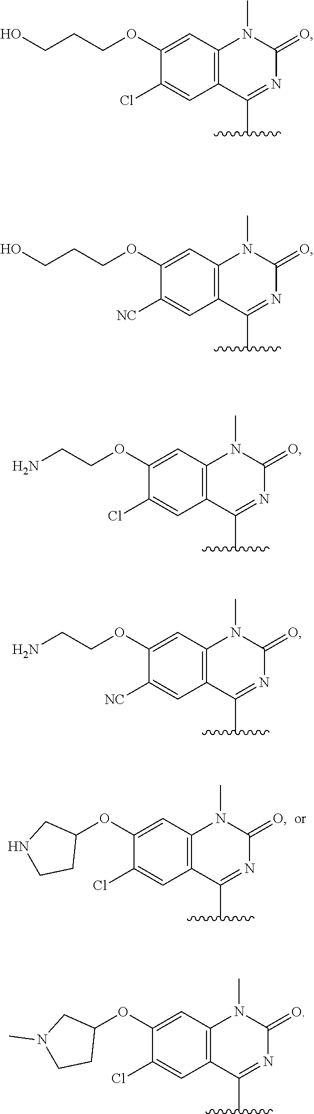

In some embodiments of a compound of Formula (I), (II), (A), (Aa) or (Ab),

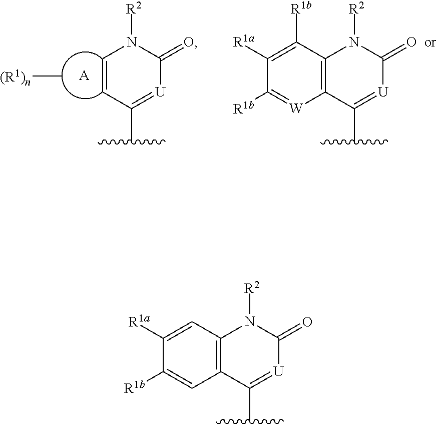

is

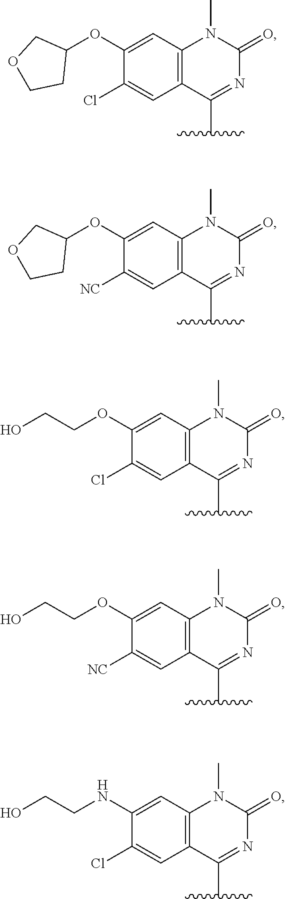 [SMILES: *c1nc(=O)n(C)c2cc(OC3CCOC3)c(Cl)cc12 ; *c1nc(=O)n(C)c2cc(OC3CCOC3)c(C#N)cc12 ; *c1nc(=O)n(C)c2cc(OCCO)c(Cl)cc12 ; *c1nc(=O)n(C)c2cc(OCCO)c(C#N)cc12 ; *c1nc(=O)n(C)c2cc(NCCO)c(Cl)cc12]

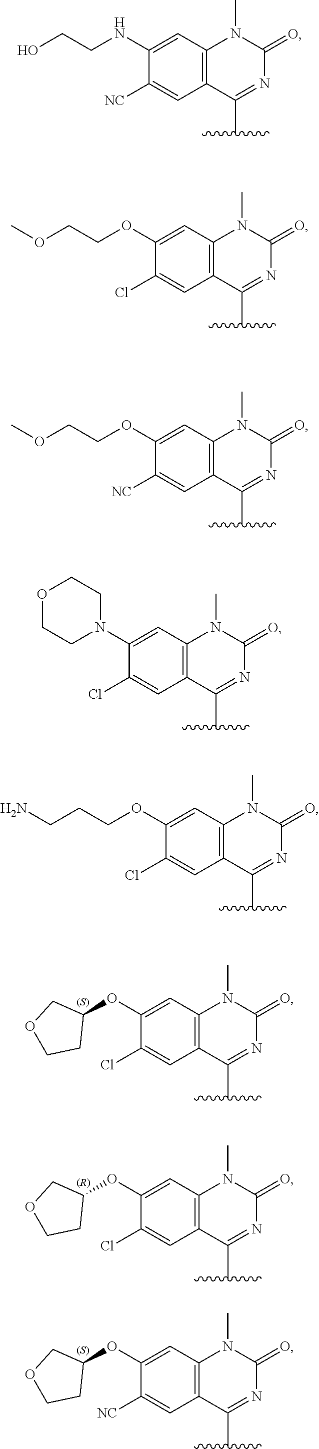

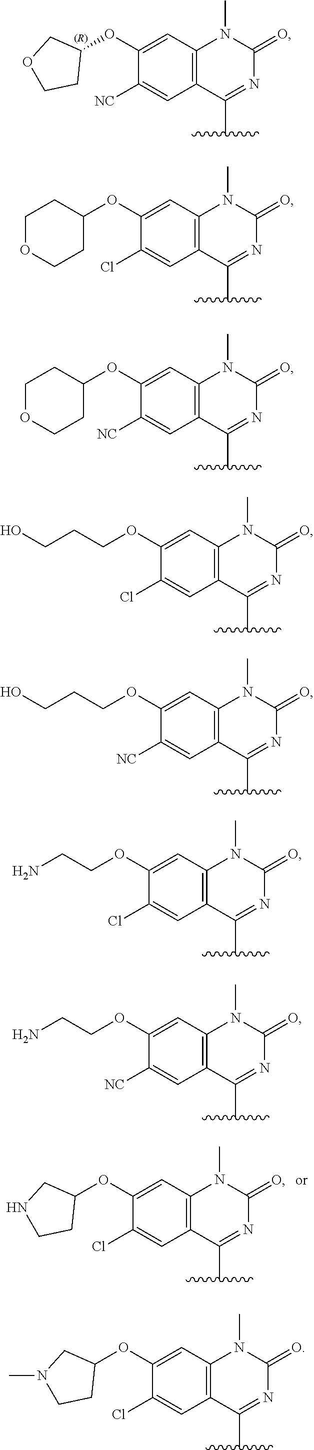

In some embodiments of a compound of Formula (I), (II), (A), (Aa) or (Ab),

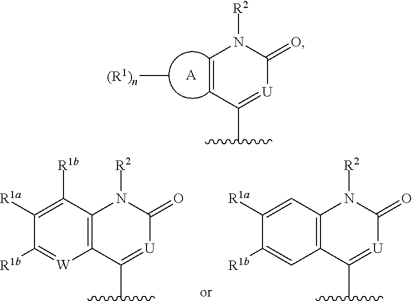

is

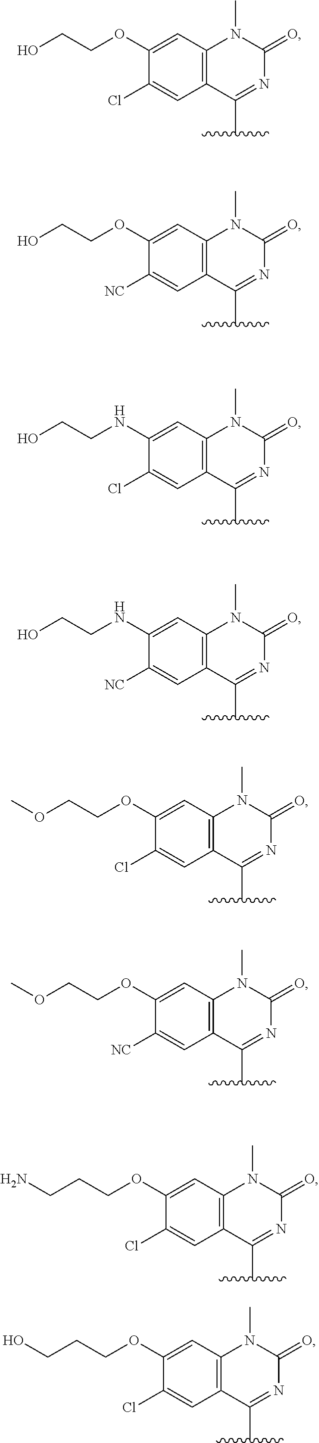

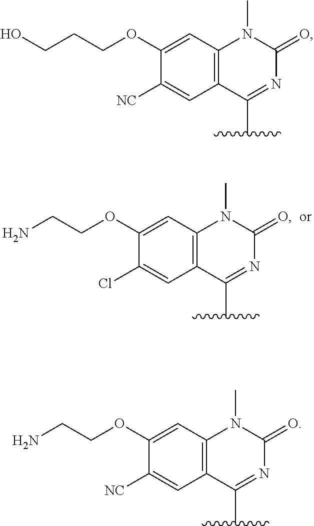

In some embodiments of a compound of Formula (I), (II), (A), (Aa) or (Ab),

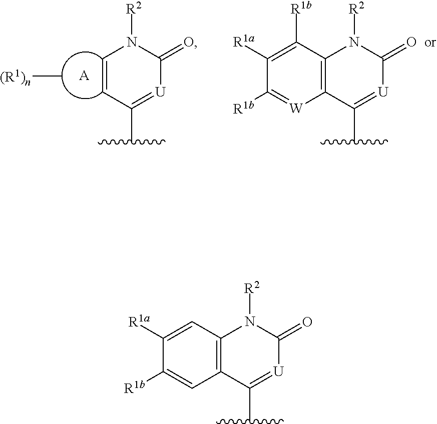

is

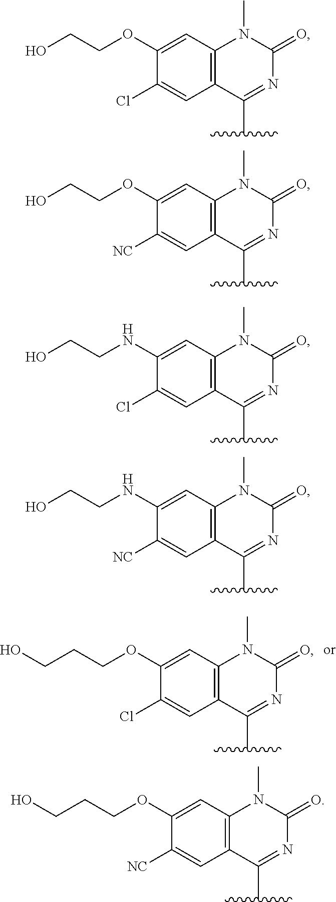

In some embodiments of a compound of Formula (I), (II), (A), (Aa) or (Ab),

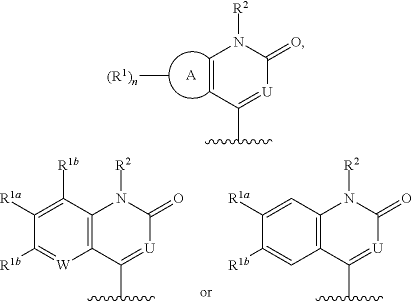

is

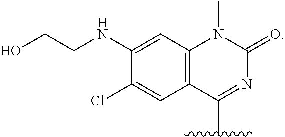 [SMILES: *c1nc(=O)n(C)c2cc(NCCO)c(Cl)cc12]

In some embodiments of a compound of Formula (I), (II), (A) (Aa) or (Ab),

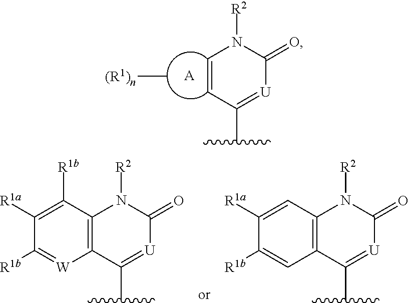

is

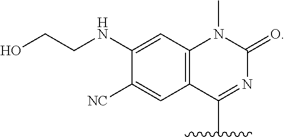 [SMILES: *c1nc(=O)n(C)c2cc(NCCO)c(C#N)cc12]

In some embodiments of a compound of Formula (I), (II), (A), (Aa) or (Ab),

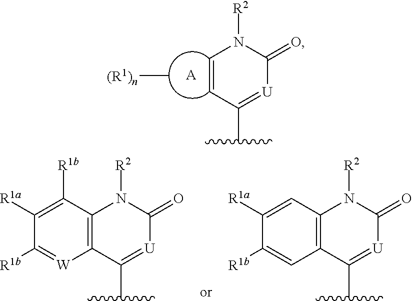

is

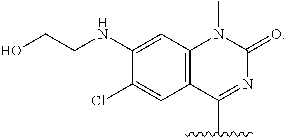 [SMILES: *c1nc(=O)n(C)c2cc(NCCO)c(Cl)cc12]

In some embodiments of a compound of Formula (I), (II), (A), (Aa) or (Ab),

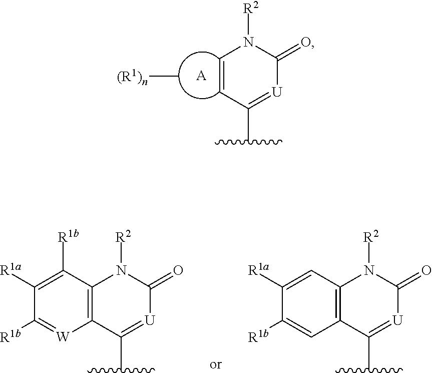

is

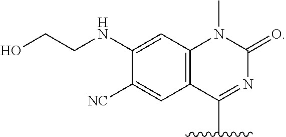 [SMILES: *c1nc(=O)n(C)c2cc(NCCO)c(C#N)cc12]

In some embodiments of a compound of Formula (I), (II), (A), (Aa) or (Ab),

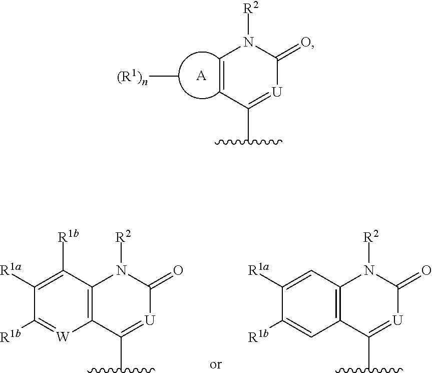

is

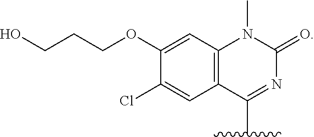 [SMILES: *c1nc(=O)n(C)c2cc(OCCCO)c(Cl)cc12]

In some embodiments of a compound of Formula (I), (II), (A), (Aa), or (Ab),

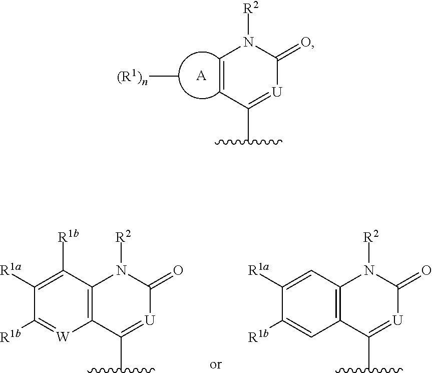

is

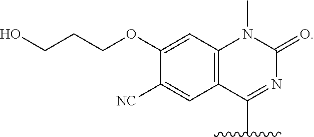 [SMILES: *c1nc(=O)n(C)c2cc(OCCCO)c(C#N)cc12]

In some embodiments of a compound of Formula (A), (Aa), (I), (Ia)-(Ig), or (II), Ring B is a 6- to 8-membered heterocycloalkyl. In some embodiments of a compound of Formula (A), (Aa), (I), (Ia)-(Ig), or (II), Ring B is a 6- to 8-membered heterocycloalkyl; comprising 1 to 3 heteroatoms selected from the group consisting of O, N, and S. In some embodiments of a compound of Formula (A), (Aa), (I), (Ia)-(Ig), or (II), Ring B is a 6- to 8-membered heterocycloalkyl; comprising 1 to 3 heteroatoms selected from the group consisting of O and N.

In some embodiments of a compound of Formula (A), (Aa), (I), (Ia)-(Ig), or (II), Ring B is a 6- to 7-membered heterocycloalkyl. In some embodiments of a compound of Formula (A), (Aa), (I), (Ia)-(Ig), or (II), Ring B is a 6- to 7-membered heterocycloalkyl comprising 1 to 3 heteroatoms selected from the group consisting of O, N, and S. In some embodiments of a compound of Formula (A), (Aa), (I), (Ia)-(Ig), or (II), Ring B is a 6- to 7-membered heterocycloalkyl comprising 1 to 3 heteroatoms selected from the group consisting of O and N.

In some embodiments of a compound of Formula (A), (Aa), (I), (Ia)-(Ig), or (II), Ring B is a 6-membered heterocycloalkyl. In some embodiments of a compound of Formula (A), (Aa), (1), (1a)-(Ig), or (II), Ring B is a 6-membered heterocycloalkyl comprising 1 to 3 heteroatoms selected from the group consisting of O, N, and S. In some embodiments of a compound of Formula (A), (Aa), (I), (Ia)-(Ig), or (II), Ring B is a 6-membered heterocycloalkyl comprising 1 to 3 heteroatoms selected from the group consisting of O and N.

In some embodiments of a compound of Formula (A), (Aa), (I), (Ia)-(Ig), or (II), Ring B is a 7-membered heterocycloalkyl. In some embodiments of a compound of Formula (A), (Aa), (1), (1a)-(Ig), or (II), Ring B is a 7-membered heterocycloalkyl comprising 1 to 3 heteroatoms selected from the group consisting of O, N, and S. In some embodiments of a compound of Formula (A), (Aa), (I), (Ia)-(Ig), or (II), Ring B is a 7-membered heterocycloalkyl comprising 1 to 3 heteroatoms selected from the group consisting of O and N.

In some embodiments of a compound of Formula (A), (Aa), (I), (Ia)-(Ig), or (II),

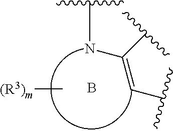

is

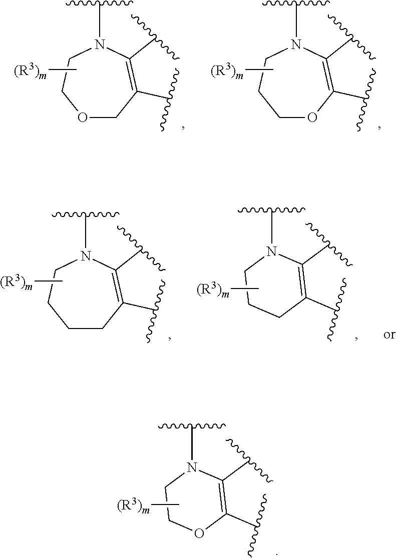

In some embodiments of a compound of Formula (A), (Aa), (I), (Ia)-(Ig), or (II),

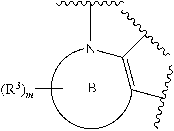

is

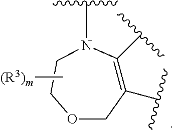

In some embodiments of a compound of Formula (A), (Aa), (I), (Ia)-(Ig), or (II), each R³ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (A), (Aa), (I), (Ia)-(Ig), or (II), each R³ is independently halogen, —CN, —OH, —OR^{a}, —NR^{c}R^{d}, C₁-C₆alkyl, or C₁-C₆haloalkyl. In some embodiments of a compound of Formula (A), (Aa), (I), (Ia)-(Ig), or (II), each R³ is independently C₁-C₆alkyl or C₁-C₆haloalkyl.

In some embodiments of a compound of Formula (A), (Aa), (I), (Ia)-(Ig), or (II), m is 0-6. In some embodiments of a compound of Formula (A), (Aa), (I), (Ia)-(Ig), or (II), m is 0-5. In some embodiments of a compound of Formula (A), (Aa), (I), (Ia)-(Ig), or (II), m is 0-4. In some embodiments of a compound of Formula (A), (Aa), (I), (Ia)-(Ig), or (II), m is 0-3. In some embodiments of a compound of Formula (A), (Aa), (I), (Ia)-(Ig), or (II), m is 0-2. In some embodiments of a compound of Formula (A), (Aa), (I), (Ia)-(Ig), or (II), m is 0 or 1. In some embodiments of a compound of Formula (A), (Aa), (I), (Ia)-(Ig), or (II), m is 1-6. In some embodiments of a compound of Formula (A), (Aa), (I), (Ia)-(Ig), or (II), m is 1-5. In some embodiments of a compound of Formula (A), (Aa), (I), (Ia)-(Ig), or (II), m is 1-4. In some embodiments of a compound of Formula (A), (Aa), (I), (Ia)-(Ig), or (IT), m is 1-3. In some embodiments of a compound of Formula (A), (Aa), (I), (Ia)-(Ig), or (II), m is 1 or 2. In some embodiments of a compound of Formula (A), (Aa), (I), (Ia)-(Ig), or (II), m is 0. In some embodiments of a compound of Formula (A), (Aa), (I), (Ia)-(Ig), or (II), m is 1. In some embodiments of a compound of Formula (A), (Aa), (I), (1a)-(Ig), or (II), m is 2. In some embodiments of a compound of Formula (A), (Aa), (I), (la)-(Ig), or (II), m is 3. In some embodiments of a compound of Formula (A), (Aa), (I), (Ia)-(Ig), or (II), m is 4. In some embodiments of a compound of Formula (A), (Aa), (I), (la)-(Ig), or (II), m is 5. In some embodiments of a compound of Formula (A), (Aa), (I), (la)-(Ig), or (IT), m is 6. In some embodiments of a compound of Formula (A), (Aa), (I), (la)-(Ig), or (11), m is 7. In some embodiments of a compound of Formula (A), (Aa), (I), (la)-(Ig), or (II), m is 5-8.

In some embodiments of a compound of Formula (A), (Aa), (I), (Ia)-(Ig), or (II),

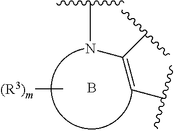

is

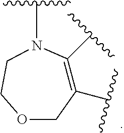 [SMILES: *C1=C(*)N(*)CCOC1]

In some embodiments of a compound of Formula (A), (Aa), (Ab), (I), (Ia)-(Ig), or (II), X is N. In some embodiments of a compound of Formula (A), (Aa), (Ab), (I), (Ia)-(Ig), or (II), X is CR^{X}.

In some embodiments of a compound of Formula (A), (Aa), (Ab), (I), (Ia)-(Ig), or (II), R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (A), (Aa), (Ab), (I), (Ia)-(Ig), or (II), R^{X} is hydrogen, halogen, —CN, C₁-C₆alkyl, or C₁-C₆haloalkyl. In some embodiments of a compound of Formula (A), (Aa), (Ab), (I), (Ia)-(Ig), or (II), R^{X} is hydrogen, halogen, or C₁-C₆alkyl. In some embodiments of a compound of Formula (A), (Aa), (Ab), (I), (Ia)-(Ig), or (II), R^{X} is hydrogen.

In some embodiments of a compound of Formula (A), (Aa), (Ab), (I), (Ia)-(Ig), or (II), Y is N. In some embodiments of a compound of Formula (A), (Aa), (Ab), (I), (Ia)-(Ig), or (II), Y is CR^{Y}.

In some embodiments of a compound of Formula (A), (Aa), (Ab), (I), (Ia)-(Ig), or (II), R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (A), (Aa), (Ab), (I), (Ia)-(Ig), or (II), R^{Y} is hydrogen, halogen, —CN, C₁-C₆alkyl, or C₁-C₆haloalkyl. In some embodiments of a compound of Formula (A), (Aa), (Ab), (I), (Ia)-(Ig), or (II), R^{Y} is hydrogen, halogen, or C₁-C₆alkyl. In some embodiments of a compound of Formula (A), (Aa), (Ab), (I), (Ia)-(Ig), or (II), R^{Y} is hydrogen.

In some embodiments of a compound of Formula (A), (Aa), (Ab), (I), (Ia)-(Ig), or (II), Z is N. In some embodiments of a compound of Formula (A), (Aa), (Ab), (I), (Ia)-(Ig), or (II), Z is CR^{Z}.

In some embodiments of a compound of Formula (A), (Aa), (Ab), (I), (Ia)-(Ig), or (II), R^{Z} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (A), (Aa), (Ab), (I), (Ia)-(Ig), or (II), R^{Z} is hydrogen, halogen, —CN, C₁-C₆alkyl, or C₁-C₆haloalkyl. In some embodiments of a compound of Formula (A), (Aa), (Ab), (I), (Ia)-(Ig), or (II), R^{Z} is hydrogen, halogen, or C₁-C₆alkyl. In some embodiments of a compound of Formula (A), (Aa), (Ab), (1), (1a)-(Ig), or (II), R^{Z} is hydrogen.

In some embodiments of a compound of Formula (A), (Aa), (I), (Ia)-(Ig), or (II),

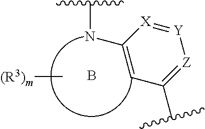

is

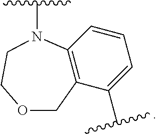 [SMILES: *c1cccc2c1COCCN2*]

In some embodiments of a compound of Formula (A), (Aa), (I), (Ia)-(Ig), or (II),

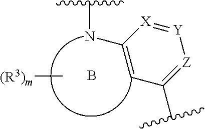

is

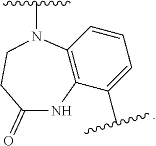 [SMILES: *c1cccc2c1NC(=O)CCN2*]

In some embodiments of a compound of Formula (A), (Aa), (I), (Ia)-(Ig), or (II),

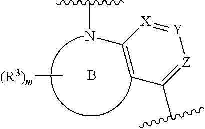

is

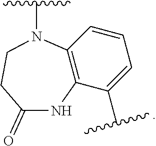 [SMILES: *c1cccc2c1NC(=O)CCN2*]

In some embodiments of a compound of Formula (A), (Aa), (I), (Ia)-(Ig), or (II),

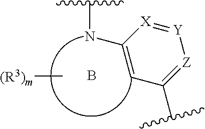

is

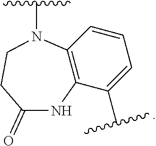 [SMILES: *c1cccc2c1NC(=O)CCN2*]

In some embodiments of a compound of Formula (A), (Aa), (Ab), (Ac), (I), (Ia)-(Ig), or (II), R⁴ is cycloalkyl, heterocycloalkyl, aryl, or heteroaryl; wherein the cycloalkyl, heterocycloalkyl, aryl, and heteroaryl is optionally substituted with one or more R^{4a}. In some embodiments of a compound of Formula (A), (Aa), (Ab), (Ac), (I), (Ia)-(Ig), or (II), R⁴ is cycloalkyl or heterocycloalkyl; wherein the cycloalkyl and heterocycloalkyl is optionally substituted with one or more R^{4a}. In some embodiments of a compound of Formula (A), (Aa), (Ab), (Ac), (I), (Ia)-(Ig), or (II), R⁴ is 3-6 membered cycloalkyl optionally substituted with one or more R^{4a}. In some embodiments of a compound of Formula (A), (Aa), (Ab), (Ac), (I), (Ia)-(Ig), or (II), R⁴ is cyclopropyl, cyclobutyl, cyclopentyl, or cyclohexyl; each optionally substituted with one or more R^{4a}. In some embodiments of a compound of Formula (A), (Aa), (Ab), (Ac), (I), (Ia)-(Ig), or (II), R⁴ is cyclopropyl optionally substituted with one or more R^{4a}. In some embodiments of a compound of Formula (A), (Aa), (Ab), (Ac), (I), (Ia)-(Ig), or (II), R⁴ is

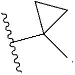 [SMILES: *C1(C)CC1]

In some embodiments of a compound of Formula (A), (Aa), (Ab), (Ac), (I), (Ia)-(Ig), or (II), R⁴ is

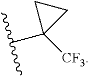 [SMILES: *C1(C(F)(F)F)CC1]

In some embodiments of a compound of Formula (A), (Aa), (Ab), (Ac), (I), (Ia)-(Ig), or (II), R⁴ is hydrogen. In some embodiments of a compound of Formula (A), (Aa), (Ab), (Ac), (I), (Ia)-(Ig), or (II), R⁴ is hydrogen, or C₁-C₆alkyl.

In some embodiments of a compound of Formula (A), (Aa), (Ab), (Ac), (I), (Ia)-(Ig), or (II), R⁴ is C₁-C₆alkyl, C₁-C₆haloalkyl, C₁-C₆hydroxyalkyl, C₁-C₆aminoalkyl, C₁-C₆heteroalkyl, 3-6 membered 3- to 6-membered cycloalkyl, 5- to 6-membered heterocycloalkyl, phenyl, or 5- to 6-membered heteroaryl; wherein the alkyl, cycloalkyl, heterocycloalkyl, aryl, and heteroaryl is optionally substituted with one or more R^{4a}.

In some embodiments of a compound of Formula (A), (Aa), (Ab), (Ac), (I), (Ia)-(Ig), or (II), each R^{4a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, alkylene, cycloalkyl, and heterocycloalkyl is optionally and independently substituted with one or more R.

In some embodiments of a compound of Formula (A), (Aa), (Ab), (Ac), (I), (Ia)-(Ig), or (II), each R^{4a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, 3- to 6-membered cycloalkyl, or 5- to 6-membered heterocycloalkyl; wherein each alkyl, cycloalkyl, and heterocycloalkyl is optionally and independently substituted with one or more R.

In some embodiments of a compound of Formula (A), (Aa), (Ab), (Ac), (I), (Ia)-(Ig), or (II), each R^{4a} is independently halogen, —CN, —OH, —OR^{a}, —NR^{c}R^{d}, C₁-C₆alkyl, or C₁-C₆haloalkyl; wherein each alkyl is optionally and independently substituted with one or more R. In some embodiments of a compound of Formula (A), (Aa), (Ab), (Ac), (I), (Ia)-(Ig), or (II), each R^{4a} is independently halogen, —CN, —OH, —OR^{a}, —NR^{c}R^{d}, C₁-C₆alkyl, or C₁-C₆haloalkyl.

In some embodiments of a compound of Formula (A), (Aa), (Ab), (Ac), (I), (Ia)-(Ig), or (II), each R^{4a} is independently C₁-C₆alkyl or C₁-C₆haloalkyl. In some embodiments of a compound of Formula (A), (Aa), (Ab), (Ac), (I), (Ia)-(Ig), or (II), each R^{4a} is independently C₁-C₆haloalkyl. In some embodiments of a compound of Formula (A), (Aa), (Ab), (Ac), (I), (Ia)-(Ig), or (II), each R^{4a} is independently C₁-C₃haloalkyl. In some embodiments of a compound of Formula (A), (Aa), (Ab), (Ac), (I), (Ia)-(Ig), or (II), each R^{4a} is independently CF₃.

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, cycloalkyl, heterocycloalkyl, aryl, and heteroaryl is independently optionally substituted with one or more R. In some embodiments of a compound disclosed herein, each R^{a} is independently C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{a} is independently C₁-C₆alkyl, C₁-C₆haloalkyl, or cycloalkyl, heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, cycloalkyl, heterocycloalkyl, aryl, and heteroaryl is independently optionally substituted with one or more R. In some embodiments of a compound disclosed herein, each R^{b} is independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{b} is independently hydrogen, C₁-C₆alkyl, C₁-C₆haloalkyl, or cycloalkyl, heterocycloalkyl. In some embodiments of a compound disclosed herein, each R^{b} is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, cycloalkyl, heterocycloalkyl, aryl, and heteroaryl is independently optionally substituted with one or more R. In some embodiments of a compound disclosed herein, each R^{c} and R^{d} are independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{c} and R^{d} are independently hydrogen, C₁-C₆alkyl, C₁-C₆haloalkyl, or cycloalkyl, heterocycloalkyl. In some embodiments of a compound disclosed herein, each R^{c} and R^{d} are independently hydrogen, C₁-C₆alkyl or C₁-C₆haloalkyl. In some embodiments of a compound disclosed herein, each R^{C} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R on the same atom form an oxo. In some embodiments of a compound disclosed herein, each R is independently halogen, —CN, —OH, —NH₂, —NHCH₃, —N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R on the same atom form an oxo. In some embodiments of a compound disclosed herein, each R is independently halogen, —CN, —OH, C₁-C₆alkyl, C₁-C₆alkoxy, or C₁-C₆haloalkyl; or two R on the same atom form an oxo. In some embodiments of a compound disclosed herein, each R is independently halogen, —CN, —OH, or C₁-C₆alkyl; or two R on the same atom form an oxo. In some embodiments of a compound disclosed herein, each R is independently halogen, —OH, or C₁-C₆alkyl. In some embodiments of a compound disclosed herein, each R is independently halogen or C₁-C₆alkyl. In some embodiments of a compound disclosed herein, each R is independently halogen.

In some embodiments of a compound disclosed herein, one or more of R, R¹, R², R³, R⁴, R^{4a}, R⁵, R^{X}, R^{Y}, R^{Z}, R^{a}, R^{b}, R^{c}, and R^{d} groups comprise deuterium at a percentage higher than the natural abundance of deuterium.

In some embodiments of a compound disclosed herein, one or more ¹H are replaced with one or more deuteriums in one or more of the following groups R, R¹, R², R³, R⁴, R^{4a}, R⁵, R^{X}, R^{Y}, R^{Z}, R^{a}, R^{b}, R^{c}, and R^{d}.

In some embodiments of a compound disclosed herein, the abundance of deuterium in each of R, R¹, R², R³, R⁴, R^{4a}, R⁵, R^{X}, R^{Y}, R^{Z}, R^{a}, R^{b}, R^{c}, and R^{d} is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more ¹H of Ring A or Ring B are replaced with one or more deuteriums.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt thereof, is one of the compounds in Table 1.

TABLE 1

| Example | Structure |
|---|---|
| 1 | 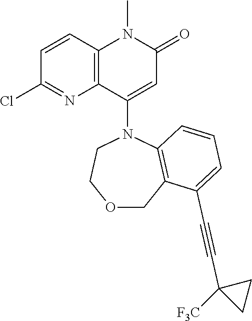 [SMILES: Cn1c(=O)cc(N2CCOCc3c(C#CC4(C(F)(F)F)CC4)cccc32)c2nc(Cl)ccc21] |
| 2 | 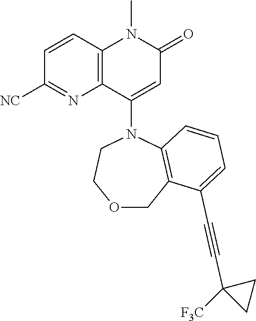 [SMILES: Cn1c(=O)cc(N2CCOCc3c(C#CC4(C(F)(F)F)CC4)cccc32)c2nc(C#N)ccc21] |
| 3 | 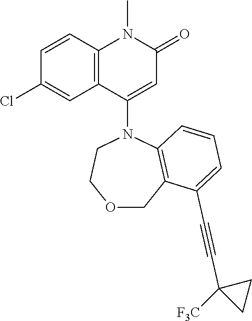 [SMILES: Cn1c(=O)cc(N2CCOCc3c(C#CC4(C(F)(F)F)CC4)cccc32)c2cc(Cl)ccc21] |
| 4 | 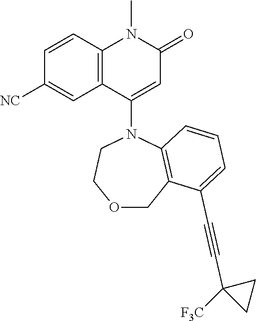 [SMILES: Cn1c(=O)cc(N2CCOCc3c(C#CC4(C(F)(F)F)CC4)cccc32)c2cc(C#N)ccc21] |
| 5 | 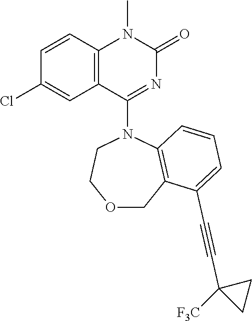 [SMILES: Cn1c(=O)nc(N2CCOCc3c(C#CC4(C(F)(F)F)CC4)cccc32)c2cc(Cl)ccc21] |
| 6 | 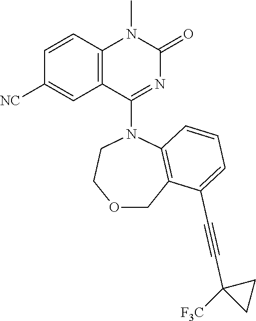 [SMILES: Cn1c(=O)nc(N2CCOCc3c(C#CC4(C(F)(F)F)CC4)cccc32)c2cc(C#N)ccc21] |
| 7 | 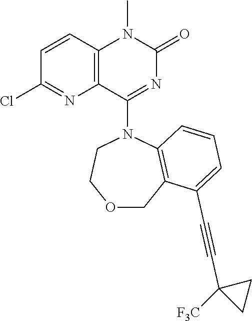 [SMILES: Cn1c(=O)nc(N2CCOCc3c(C#CC4(C(F)(F)F)CC4)cccc32)c2nc(Cl)ccc21] |
| 8 | 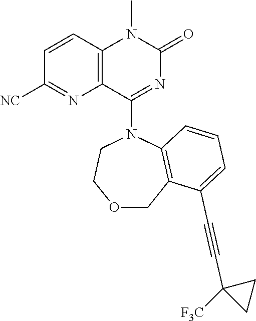 [SMILES: Cn1c(=O)nc(N2CCOCc3c(C#CC4(C(F)(F)F)CC4)cccc32)c2nc(C#N)ccc21] |
| 9 | 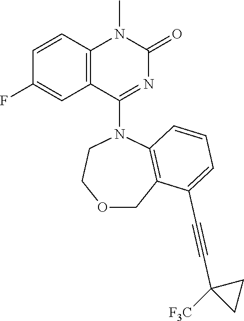 [SMILES: Cn1c(=O)nc(N2CCOCc3c(C#CC4(C(F)(F)F)CC4)cccc32)c2cc(F)ccc21] |
| 10 | 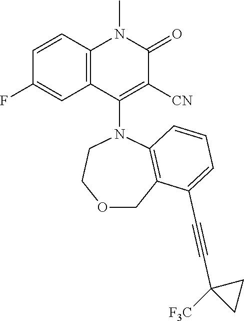 [SMILES: Cn1c(=O)c(C#N)c(N2CCOCc3c(C#CC4(C(F)(F)F)CC4)cccc32)c2cc(F)ccc21] |
| 11 | 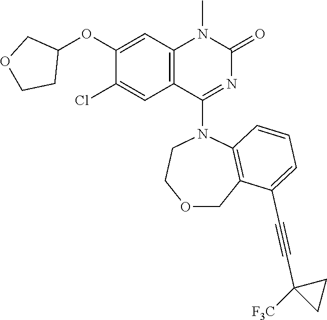 [SMILES: Cn1c(=O)nc(N2CCOCc3c(C#CC4(C(F)(F)F)CC4)cccc32)c2cc(Cl)c(OC3CCOC3)cc21] |
| 12 | 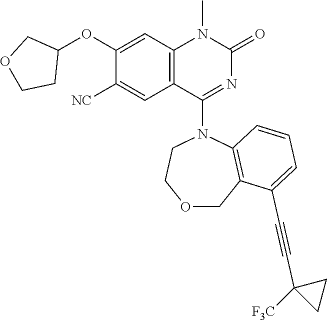 [SMILES: Cn1c(=O)nc(N2CCOCc3c(C#CC4(C(F)(F)F)CC4)cccc32)c2cc(C#N)c(OC3CCOC3)cc21] |
| 13 | 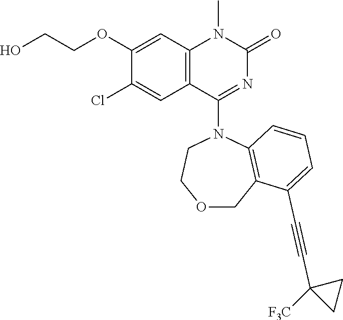 [SMILES: Cn1c(=O)nc(N2CCOCc3c(C#CC4(C(F)(F)F)CC4)cccc32)c2cc(Cl)c(OCCO)cc21] |
| 14 | 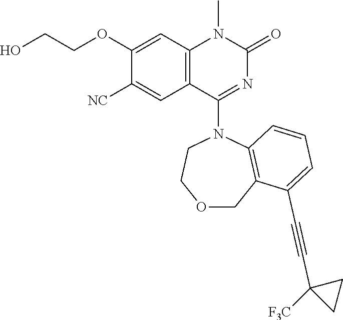 [SMILES: Cn1c(=O)nc(N2CCOCc3c(C#CC4(C(F)(F)F)CC4)cccc32)c2cc(C#N)c(OCCO)cc21] |
| 15 | 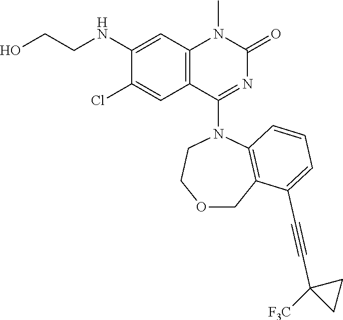 [SMILES: Cn1c(=O)nc(N2CCOCc3c(C#CC4(C(F)(F)F)CC4)cccc32)c2cc(Cl)c(NCCO)cc21] |
| 16 | 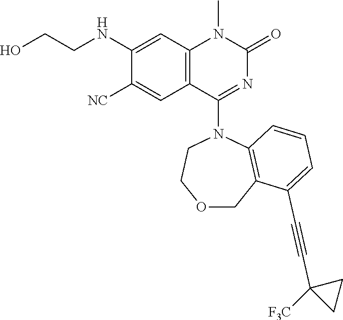 [SMILES: Cn1c(=O)nc(N2CCOCc3c(C#CC4(C(F)(F)F)CC4)cccc32)c2cc(C#N)c(NCCO)cc21] |
| 17 | 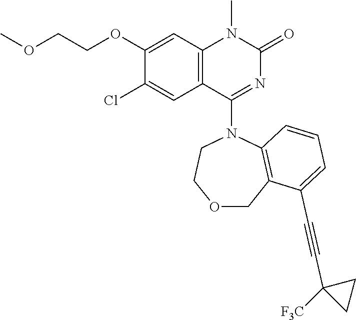 [SMILES: COCCOc1cc2c(cc1Cl)c(N1CCOCc3c(C#CC4(C(F)(F)F)CC4)cccc31)nc(=O)n2C] |
| 18 | 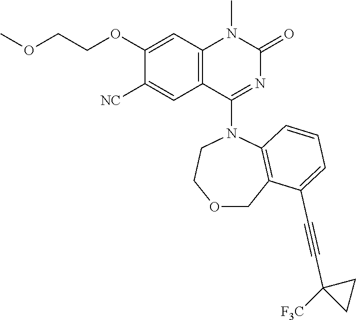 [SMILES: COCCOc1cc2c(cc1C#N)c(N1CCOCc3c(C#CC4(C(F)(F)F)CC4)cccc31)nc(=O)n2C] |
| 19 | 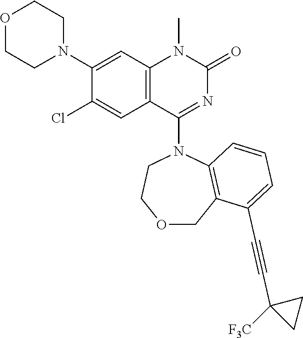 [SMILES: Cn1c(=O)nc(N2CCOCc3c(C#CC4(C(F)(F)F)CC4)cccc32)c2cc(Cl)c(N3CCOCC3)cc21] |
| 20 | 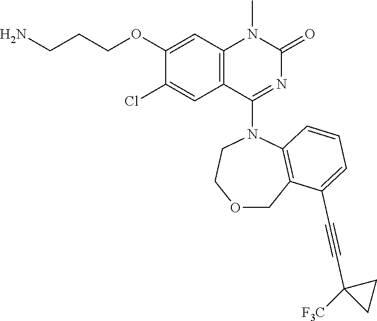 [SMILES: Cn1c(=O)nc(N2CCOCc3c(C#CC4(C(F)(F)F)CC4)cccc32)c2cc(Cl)c(OCCCN)cc21] |
| 21 | 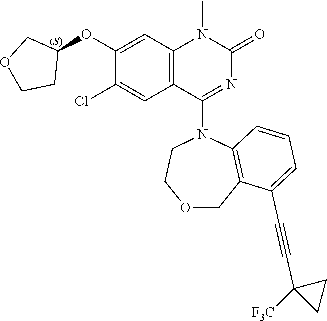 |
| 22 | 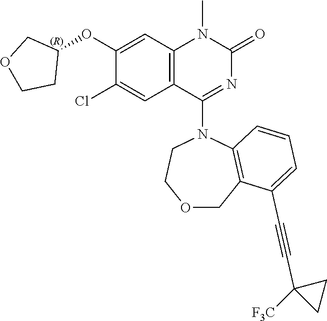 |
| 23 | 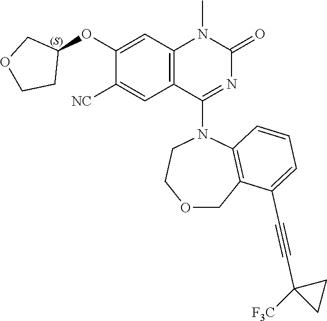 |
| 24 | 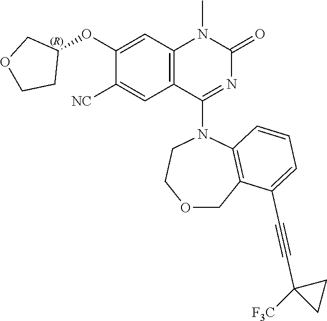 |
| 25 | 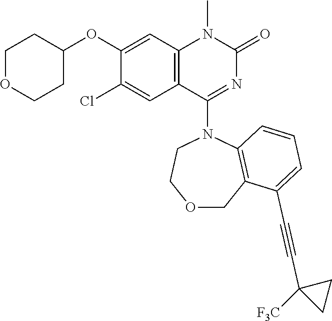 [SMILES: Cn1c(=O)nc(N2CCOCc3c(C#CC4(C(F)(F)F)CC4)cccc32)c2cc(Cl)c(OC3CCOCC3)cc21] |
| 26 | 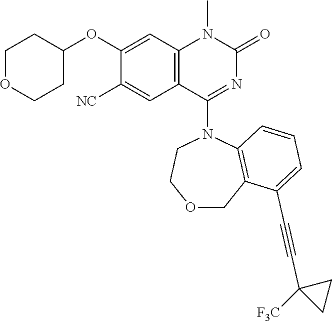 [SMILES: Cn1c(=O)nc(N2CCOCc3c(C#CC4(C(F)(F)F)CC4)cccc32)c2cc(C#N)c(OC3CCOCC3)cc21] |
| 27 | 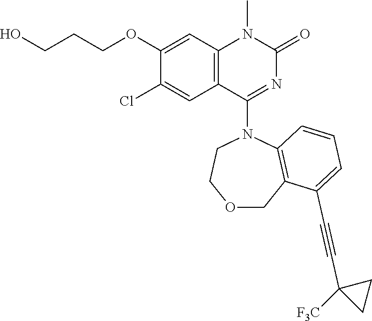 [SMILES: Cn1c(=O)nc(N2CCOCc3c(C#CC4(C(F)(F)F)CC4)cccc32)c2cc(Cl)c(OCCCO)cc21] |
| 28 | 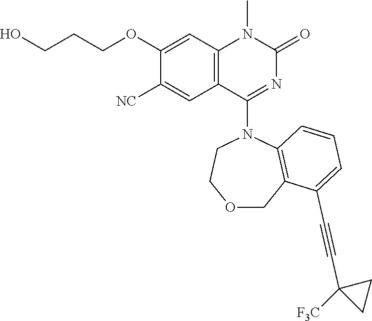 [SMILES: Cn1c(=O)nc(N2CCOCc3c(C#CC4(C(F)(F)F)CC4)cccc32)c2cc(C#N)c(OCCCO)cc21] |
| 29 | 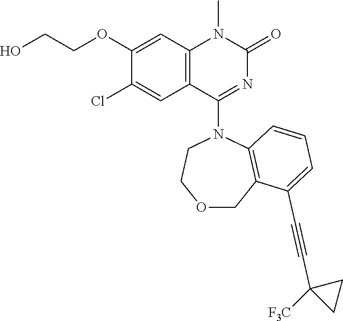 [SMILES: Cn1c(=O)nc(N2CCOCc3c(C#CC4(C(F)(F)F)CC4)cccc32)c2cc(Cl)c(OCCO)cc21] |
| 30 | 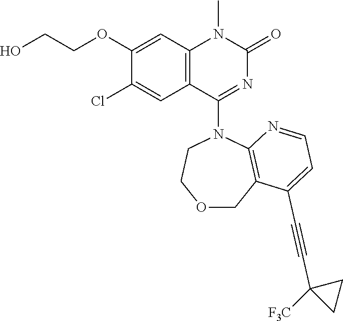 [SMILES: Cn1c(=O)nc(N2CCOCc3c(C#CC4(C(F)(F)F)CC4)ccnc32)c2cc(Cl)c(OCCO)cc21] |
| 31 | 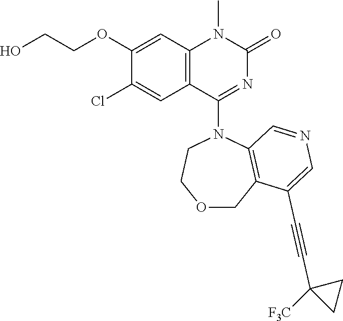 [SMILES: Cn1c(=O)nc(N2CCOCc3c(C#CC4(C(F)(F)F)CC4)cncc32)c2cc(Cl)c(OCCO)cc21] |
| 32 | 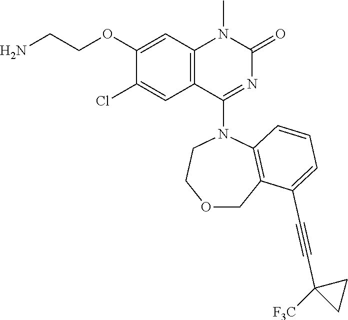 [SMILES: Cn1c(=O)nc(N2CCOCc3c(C#CC4(C(F)(F)F)CC4)cccc32)c2cc(Cl)c(OCCN)cc21] |
| 33 | 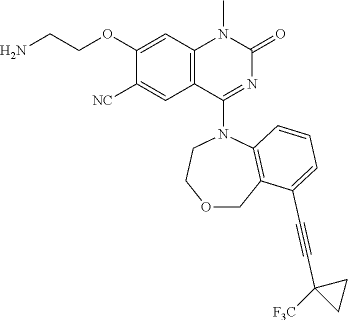 [SMILES: Cn1c(=O)nc(N2CCOCc3c(C#CC4(C(F)(F)F)CC4)cccc32)c2cc(C#N)c(OCCN)cc21] |
| 34 | 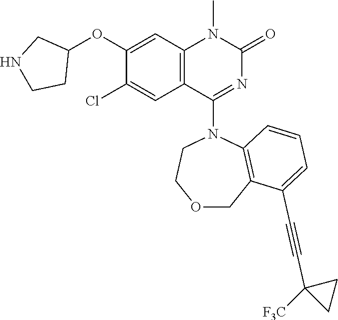 [SMILES: Cn1c(=O)nc(N2CCOCc3c(C#CC4(C(F)(F)F)CC4)cccc32)c2cc(Cl)c(OC3CCNC3)cc21] |
| 35 | 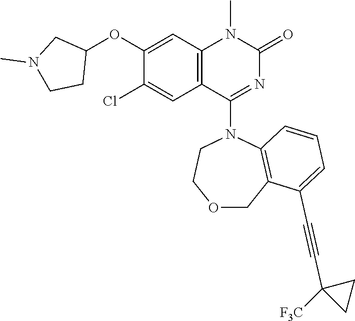 [SMILES: CN1CCC(Oc2cc3c(cc2Cl)c(N2CCOCc4c(C#CC5(C(F)(F)F)CC5)cccc42)nc(=O)n3C)C1] |
| 36 | 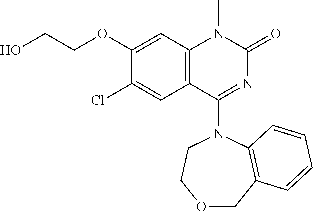 [SMILES: Cn1c(=O)nc(N2CCOCc3ccccc32)c2cc(Cl)c(OCCO)cc21] |
| 37 | 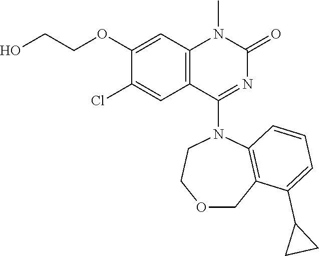 [SMILES: Cn1c(=O)nc(N2CCOCc3c(C4CC4)cccc32)c2cc(Cl)c(OCCO)cc21] |
| 38 | 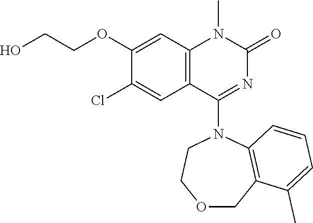 [SMILES: Cc1cccc2c1COCCN2c1nc(=O)n(C)c2cc(OCCO)c(Cl)cc12] |
| 39 | 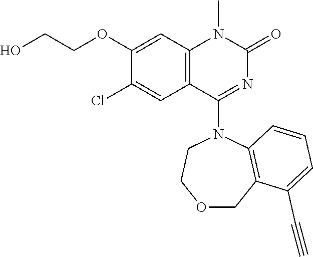 [SMILES: C#Cc1cccc2c1COCCN2c1nc(=O)n(C)c2cc(OCCO)c(Cl)cc12] |
| 40 | 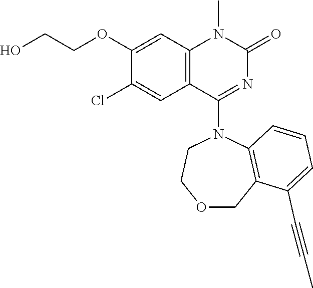 [SMILES: CC#Cc1cccc2c1COCCN2c1nc(=O)n(C)c2cc(OCCO)c(Cl)cc12] |
| 41 | 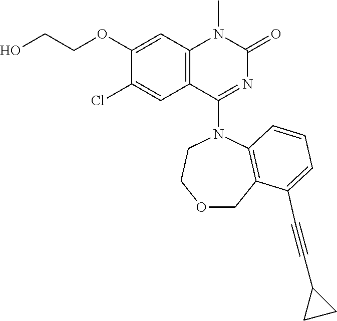 [SMILES: Cn1c(=O)nc(N2CCOCc3c(C#CC4CC4)cccc32)c2cc(Cl)c(OCCO)cc21] |
| 42 | 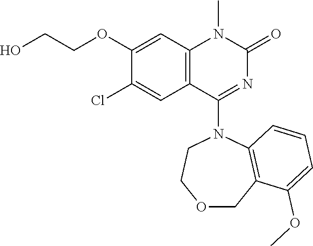 [SMILES: COc1cccc2c1COCCN2c1nc(=O)n(C)c2cc(OCCO)c(Cl)cc12] |
| 43 | 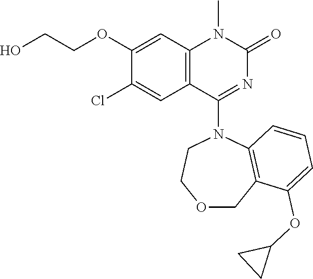 [SMILES: Cn1c(=O)nc(N2CCOCc3c(OC4CC4)cccc32)c2cc(Cl)c(OCCO)cc21] |
| 44 | 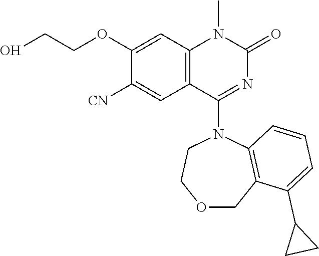 |
| 45 | 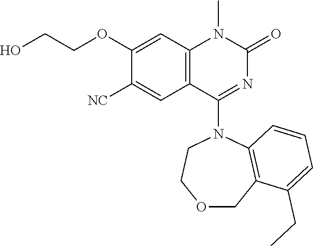 [SMILES: CCc1cccc2c1COCCN2c1nc(=O)n(C)c2cc(OCCO)c(C#N)cc12] |
| 46 | 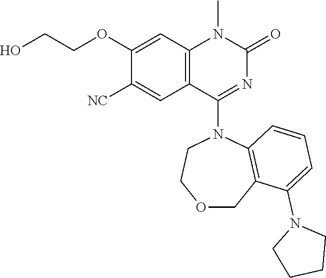 [SMILES: Cn1c(=O)nc(N2CCOCc3c(N4CCCC4)cccc32)c2cc(C#N)c(OCCO)cc21] |
| 47 | 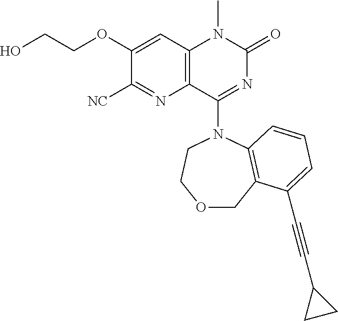 [SMILES: Cn1c(=O)nc(N2CCOCc3c(C#CC4CC4)cccc32)c2nc(C#N)c(OCCO)cc21] |

In some embodiments the compound disclosed herein, or a pharmaceutically acceptable salt thereof, is one of the compounds in Table 2.

TABLE 2
Structure

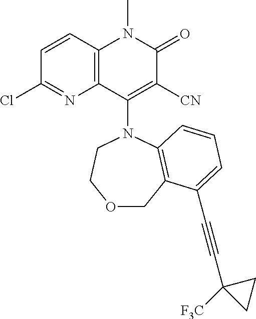 [SMILES: Cn1c(=O)c(C#N)c(N2CCOCc3c(C#CC4(C(F)(F)F)CC4)cccc32)c2nc(Cl)ccc21]

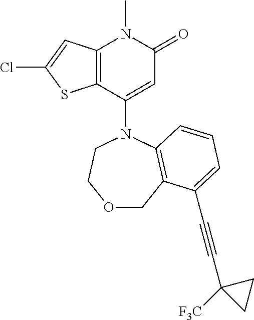 [SMILES: Cn1c(=O)cc(N2CCOCc3c(C#CC4(C(F)(F)F)CC4)cccc32)c2sc(Cl)cc21]

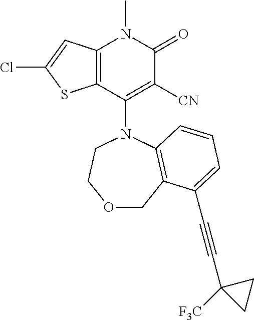 [SMILES: Cn1c(=O)c(C#N)c(N2CCOCc3c(C#CC4(C(F)(F)F)CC4)cccc32)c2sc(Cl)cc21]

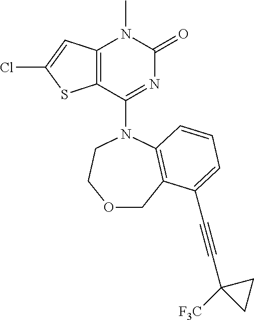 [SMILES: Cn1c(=O)nc(N2CCOCc3c(C#CC4(C(F)(F)F)CC4)cccc32)c2sc(Cl)cc21]

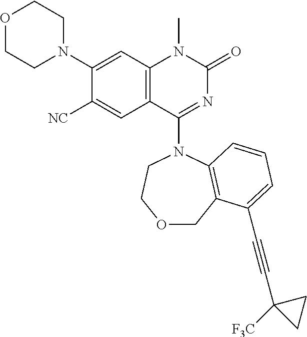 [SMILES: Cn1c(=O)nc(N2CCOCc3c(C#CC4(C(F)(F)F)CC4)cccc32)c2cc(C#N)c(N3CCOCC3)cc21]

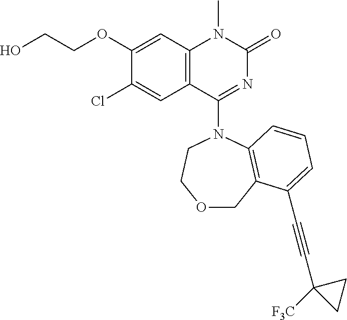 [SMILES: Cn1c(=O)nc(N2CCOCc3c(C#CC4(C(F)(F)F)CC4)cccc32)c2cc(Cl)c(OCCO)cc21]

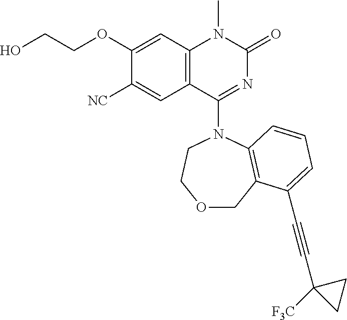 [SMILES: Cn1c(=O)nc(N2CCOCc3c(C#CC4(C(F)(F)F)CC4)cccc32)c2cc(C#N)c(OCCO)cc21]

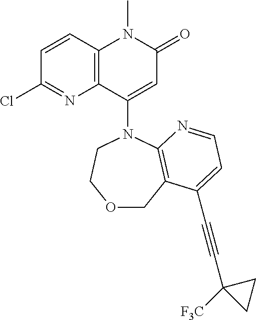 [SMILES: Cn1c(=O)cc(N2CCOCc3c(C#CC4(C(F)(F)F)CC4)ccnc32)c2nc(Cl)ccc21]

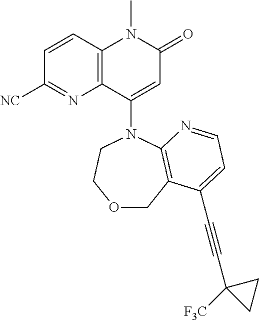 [SMILES: Cn1c(=O)cc(N2CCOCc3c(C#CC4(C(F)(F)F)CC4)ccnc32)c2nc(C#N)ccc21]

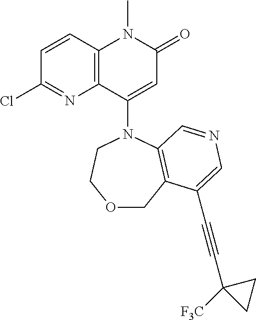 [SMILES: Cn1c(=O)cc(N2CCOCc3c(C#CC4(C(F)(F)F)CC4)cncc32)c2nc(Cl)ccc21]

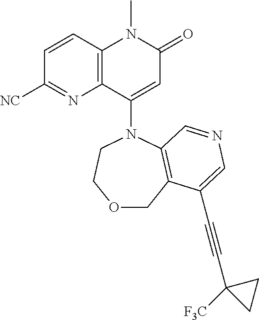 [SMILES: Cn1c(=O)cc(N2CCOCc3c(C#CC4(C(F)(F)F)CC4)cncc32)c2nc(C#N)ccc21]

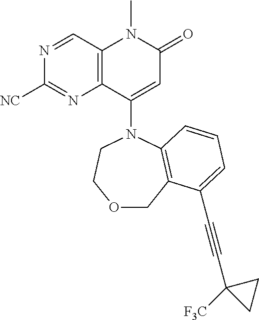 [SMILES: Cn1c(=O)cc(N2CCOCc3c(C#CC4(C(F)(F)F)CC4)cccc32)c2nc(C#N)ncc21]

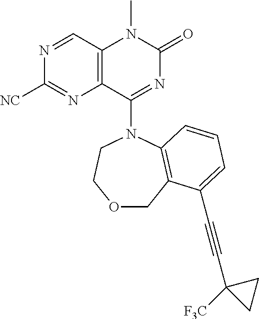 [SMILES: Cn1c(=O)nc(N2CCOCc3c(C#CC4(C(F)(F)F)CC4)cccc32)c2nc(C#N)ncc21]

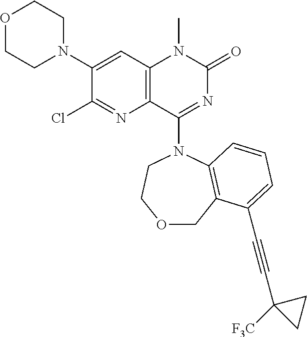 [SMILES: Cn1c(=O)nc(N2CCOCc3c(C#CC4(C(F)(F)F)CC4)cccc32)c2nc(Cl)c(N3CCOCC3)cc21]

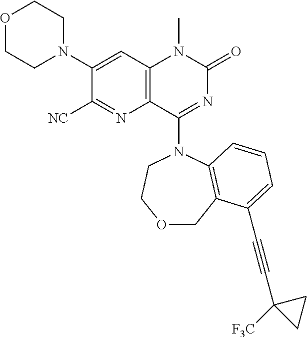 [SMILES: Cn1c(=O)nc(N2CCOCc3c(C#CC4(C(F)(F)F)CC4)cccc32)c2nc(C#N)c(N3CCOCC3)cc21]

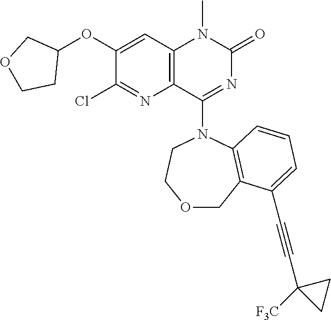 [SMILES: Cn1c(=O)nc(N2CCOCc3c(C#CC4(C(F)(F)F)CC4)cccc32)c2nc(Cl)c(OC3CCOC3)cc21]

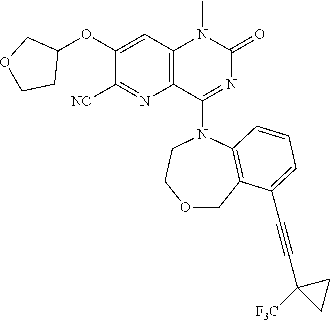 [SMILES: Cn1c(=O)nc(N2CCOCc3c(C#CC4(C(F)(F)F)CC4)cccc32)c2nc(C#N)c(OC3CCOC3)cc21]

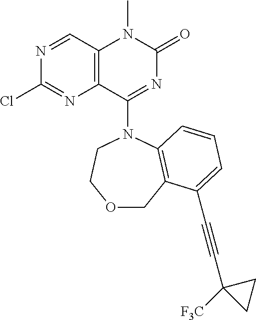 [SMILES: Cn1c(=O)nc(N2CCOCc3c(C#CC4(C(F)(F)F)CC4)cccc32)c2nc(Cl)ncc21]

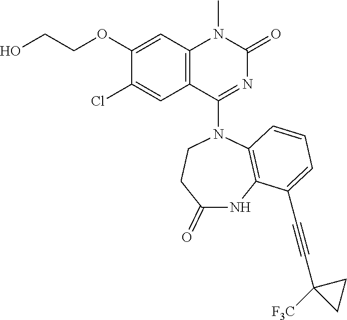 [SMILES: Cn1c(=O)nc(N2CCC(=O)Nc3c(C#CC4(C(F)(F)F)CC4)cccc32)c2cc(Cl)c(OCCO)cc21]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Isotopically Enriched Compounds

Unless otherwise stated, compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford some therapeutic advantages, such as increased in vivo half-life and/or exposure, or may provide a compound useful for investigating in vivo routes of drug elimination and metabolism.

For example, the compounds described herein may be artificially enriched in one or more particular isotopes. In some embodiments, the compounds described herein may be artificially enriched in one or more isotopes that are not predominantly found in nature. In some embodiments, the compounds described herein may be artificially enriched in one or more isotopes selected from deuterium (²H), tritium (³H), iodine-125 (¹²⁵I) or carbon-14 (¹⁴C). In some embodiments, the compounds described herein are artificially enriched in one or more isotopes selected from ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹³¹I, and ¹²⁵I. In some embodiments, the abundance of the enriched isotopes is independently at least 1%, at least 10%, at least 20%, at least 30%, at least 40%, at least 50%, at least 60%, at least 70%, at least 80%, at least 90%, or 100% by molar.

In some embodiments, the compound is deuterated in at least one position. In some embodiments, the compounds disclosed herein have some or all of the ¹H atoms replaced with ²H atoms.

The methods of synthesis for deuterium-containing compounds are known in the art and include, by way of non-limiting example only, the procedure described in U.S. Pat. Nos. 5,846,514 and 6,334,997, and the following synthetic methods. For example, deuterium substituted compounds may be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of treating a disease modulated by DGK in a subject in need thereof, comprising administering to the subject a therapeutically affective amount of a compound, or a pharmaceutically acceptable salt thereof, disclosed herein.

Disclosed herein are methods of treating a disease in a subject in need thereof, comprising administering to the subject a therapeutically affective amount of a compound, or a pharmaceutically acceptable salt thereof, disclosed herein. In some embodiments, the disease is cancer.

Disclosed herein are methods of treating a disease modulated by DGKalpha in a subject in need thereof, comprising administering to the subject a therapeutically affective amount of a compound, or a pharmaceutically acceptable salt thereof, disclosed herein. In some embodiments, the disease is cancer or a viral infection.

In some embodiments, the cancer is a solid tumor. In some embodiments, the cancer is a hematological cancer. In some embodiments, the cancer is breast cancer, cervical cancer, colon cancer, head and neck cancer, leukemia, liver cancer, lung cancer, lymphoma, melanoma, ovarian cancer, pancreatic cancer, prostate cancer, renal cancer, thyroid cancer, or urinary track cancer.

In some embodiments, the viral infection is an HIV infection, an hepatitis B virus infection, an hepatitis C virus infection, a human papilloma virus infection, a cytomegalovirus infection, herpes simplex virus infection, Epstein-Barr virus infection, or a varicella zoster virus infection.

Disclosed herein is a method of inhibiting the activity of at least one of diacylglycerol kinase comprising administering to the subject a therapeutically affective amount of a compound, or a pharmaceutically acceptable salt thereof, disclosed herein. In some embodiments, the diacylglycerol kinase is diacylglycerol kinase alpha (DGKalpha). In some embodiments, the diacylglycerol kinase is diacylglycerol kinase zeta (DGKzeta). Disclosed herein is a method of modulating the activity of at least one of diacylglycerol kinase selected from diacylglycerol kinase alpha (DGKalpha) and diacylglycerol kinase zeta (DGKzeta), in a subject in need thereof, the method comprising administering to the subject a compound, or a pharmaceutically acceptable salt thereof, disclosed herein. Disclosed herein is a method of modulating the activity of diacylglycerol kinase alpha (DGKalpha), in a subject in need thereof, the method comprising administering to the subject a compound, or a pharmaceutically acceptable salt thereof, disclosed herein. In some embodiments, the subject has a disease described herein. In some embodiments, the subject has cancer.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating a disease or disorder associated with DGKalpha using a compound disclosed herein, or a pharmaceutically acceptable salt thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an anti-cancer agent. In some embodiments, the anti-cancer agent is an immune checkpoint inhibitor. In some embodiments, the immune checkpoint inhibitor is an anti-CTLA-4 (Cytotoxic T lymphocyte antigen 4) antibody, an anti-PD-1 (Programmed death receptor 1) antibody, or an anti-PD-L1 (Programmed death ligand 1) antibody.

In some embodiments, the additional therapeutic agent is an anti-viral agent.

### EXAMPLES

### Example 1: 6-chloro-1-methyl-4-(6-((1-(trifluoromethyl)cyclopropyl)ethynyl)-2,3-dihydrobenzo[e][1,4]oxazepin-1(5H)-yl)-1,5-naphthyridin-2(1H)-one

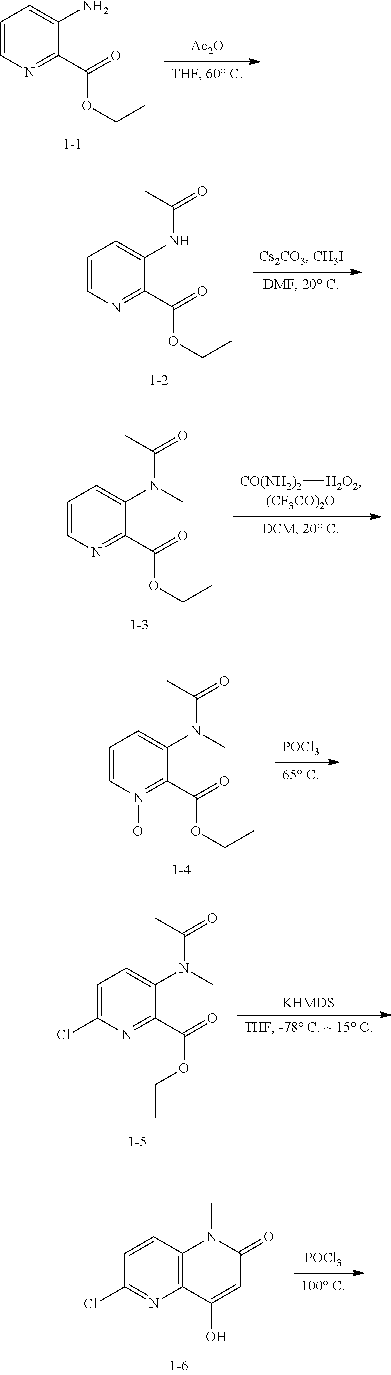

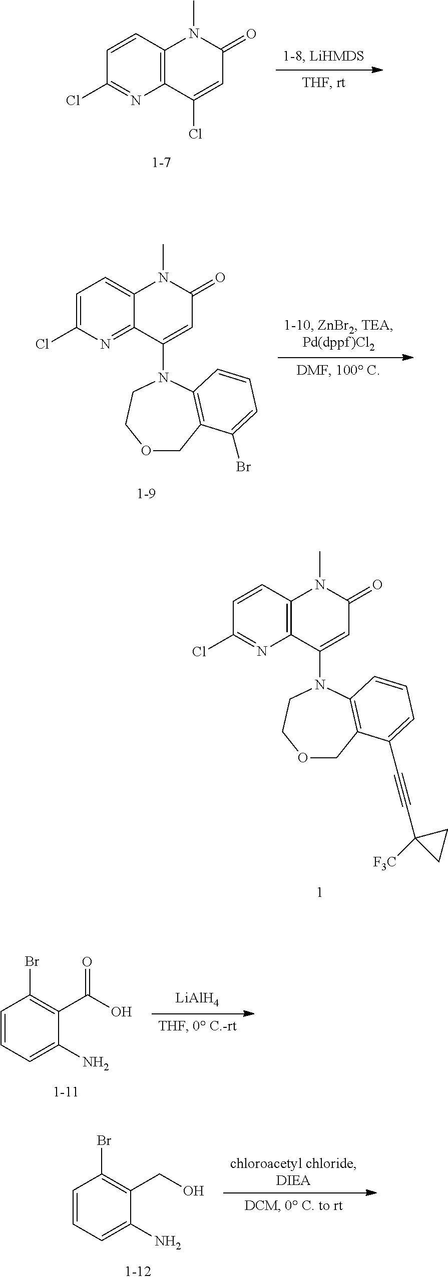

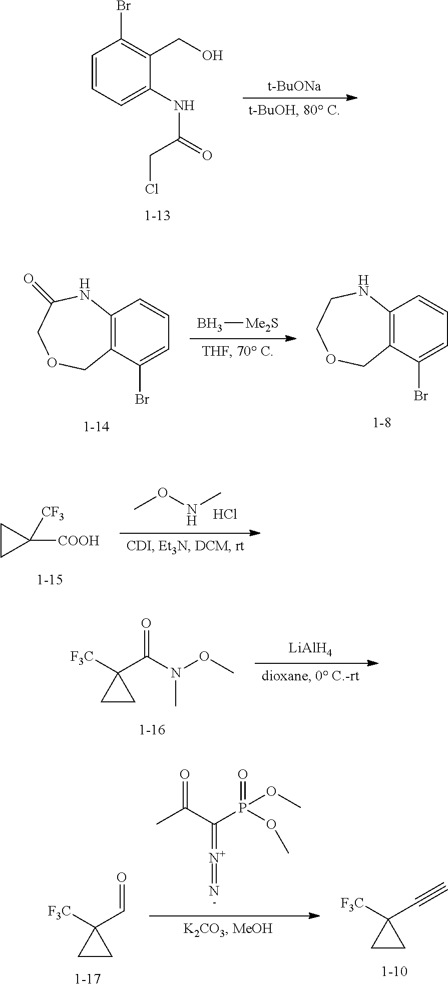

To a mixture of compound 1-1 (10 g, 60.18 mmol) in THF (100 mL) was added Ac₂O (55.29 g, 541.58 mmol) at room temperature. The reaction mixture was stirred at 60° C. under N₂ for 18 h. After cooling to room temperature, the mixture was concentrated under reduced pressure. To the resulting residue was added petroleum ether (500 mL) and the suspension was stirred for 30 min at 20° C. The mixture was filtered and the filter cake was rinsed with petroleum ether (500 mL). The filter cake was collected and dried under vacuum to give compound 1-2. LCMS: MS (ESI) m/z (M+H)⁺=209.0.

To a mixture of compound 1-2 (10.7 g, 51.39 mmol) and cesium carbonate (25.11 g, 77.08 mmol) in DMF (200 mL) was added methyl iodide (10.94 g, 77.08 mmol) at 20° C. The mixture was stirred at room temperature for 18 h. The reaction mixture was diluted with water (500 mL) and extracted with DCM (3×200 mL). The combined organic layers were washed with brine (2×100 mL), dried over Na₂SO₄, filtered, and concentrated under reduced pressure. To the resulting residue was added petroleum ether (500 mL) and the suspension was stirred for 30 min at 20° C. The mixture was filtered and the filter cake was rinsed with petroleum ether (500 mL). The filter cake was collected and dried under reduced pressure to give compound 1-3. LCMS: MS (ESI) m/z (M+H)⁺=223.1.

To a mixture of compound 1-3 (4 g, 18.00 mmol) in DCM (200 mL) at 0-5° C. was added urea hydrogen peroxide (2.54 g, 27.0 mmol), followed by trifluoroacetic anhydride (5.67 g, 27.00 mmol) slowly over 40 min. The reaction mixture solidified during the trifluoroacetic anhydride addition. After completion of the addition, the reaction mixture was stirred at 20 C for 18 h. The reaction mixture was quenched with 10% NaHCO₃ solution (100 mL) and extracted with DCM (3×100 mL). The combined organic layers were washed with brine (2×100 mL), dried over Na₂SO₄, and concentrated under reduced pressure to give compound 1-4, which was used in the next step without further purification. LCMS: MS (ESI) m/z (M+H)⁺=239.1.

A mixture of 1-4 (2 g, 8.40 mmol) in POCl₃ (15 mL) was stirred at 65° C. for 2 h. The mixture was concentrated under reduced pressure. The residue was diluted with DCM (50 mL) and adjusted to pH=8.0 with Sat. NaHCO₃ solution. The mixture was extracted with DCM (3×50 mL). The combined organic layers were dried over Na₂SO₄, filtered, and concentrated under reduced pressure to give the residue, which was purified by flash column chromatography to give 1-5, which was checked by LCMS: MS m/z (ESI) [M+H]⁺=257.2.

To a solution of KHMDS (1M in THF) (6.68 mL, 6.68 mmol) in THF (100 mL) at −78° C. was added a solution of 1-5 (1.4 g, 4.45 mmol) in THF (10 mL) dropwise under N₂. After addition, the mixture was slowly warmed up to 15° C. and stirred at the temperature for 2 h. The mixture was adjusted to pH=6.0 with 1 N HCl. The mixture was evaporated to give the crude, which was purified by flash column chromatography to give 1-6, which was checked by LCMS: MS m/z (ESI) [M+H]⁺=211.2.

A mixture of 1-6 (680 mg, 2.502 mmol) in POCl₃ (20 mL) was stirred at 100° C. for 2 h. The mixture was concentrated under reduced pressure to give the residue. The residue was diluted with DCM (50 mL) and adjusted to pH=8.0 with Sat. NaHCO₃ solution. The resulting mixture was extracted with DCM (3×50 mL). The combined organic layers were dried over Na₂SO₄, filtered, and concentrated under reduced pressure to give the crude, which was purified by flash column chromatography to give 1-7, which was checked by LCMS: MS m/z (EST) [M+H]⁺=229.1.

To a mixture of 1-11 (8.0 g, 37.03 mmol) in anhydrous THF (200 mL) at 0° C. was dropwise added lithium aluminum hydride (51.8 mL, 1.0 M in THF). After addition, the reaction mixture was allowed to warm to room temperature and stirred overnight. The mixture was quenched with water (30 mL), followed by 15% NaOH solution (7.7 mL). The mixture was stirred for another 1 hour and filtered. The filtrate was concentrated to give 1-12, which was checked by LCMS: LCMS: MS m/z (EST) [M−17]⁺=183.9.

To a mixture of chloroacetyl chloride (3.07 g, 27.22 mmol) in dry DCM (200 mL) was added 1-12 (5.0 g, 24.75 mmol) at room temperature, followed by addition of diisopropylethylamine (9.59 g, 74.24 mmol). The mixture was stirred at ambient temperature for 18 h. The mixture was quenched with saturated sodium bicarbonate. The organic layer was separated and washed with saturated NaHCO₃ solution (50 mL) and water (50 mL×2). The organic phase was dried over MgSO₄, filtered and the filtrate was evaporated to give 1-13, which was checked by LCMS: LCMS: MS m/z (ESI) [M−17]⁺=260.0. ¹H NMR (400 MHz, CDCl₃) δ 9.76 (s, 1H), 7.98 (d, J=8.1 Hz, 1H), 7.39 (dd, J=8.1, 0.9 Hz, 1H), 7.19 (t, J=8.1 Hz, 1H), 4.97 (s, 2H), 4.20 (s, 2H).

A suspension of sodium tert-butoxide (2.42 g, 25.20 mmol) in t-BuOH (80 mL) was heated at 80° C. until it turned into a clear solution. Then, 1-13 (3.90 g, 14.00 mmol) was added in one portion and the reaction was stirred at 80° C. for 2 h. The reaction mixture was cooled, poured into ice-water, and extracted with ethyl acetate. The organic layer were washed with brine, dried over anhydrous sodium sulfate, and concentrated. The resulting residue was purified by flash column chromatography to give 1-14, which was checked by LCMS: LCMS: MS m/z (ESI) [M+H]⁺=241.9.

To a mixture of 1-14 (3.3 g, 13.63 mmol) in anhydrous THF (200 mL) at 0° C., BH₃-Me₂S (20.4 mL, 2.0 M) was slowly added. The reaction was refluxed for 2 hour. The mixture was cooled to 0° C., and quenched by water (200 mL) slowly. The mixture was extracted with ethyl acetate, and the organic layer was concentrated. The resulting residue was purified by silico gel column chromatography to give 1-8 which was checked by LCMS: LCMS: MS m/z (ESI) [M+H]j=229.9.

To a mixture of 1-8 (226.31 mg, 0.992 mmol) in THF (8 mL) was added LiHMDS (2.977 mL, 1 M) at RT under N₂. The mixture was stirred at 20° C. for 10 min under N₂. Then 1-7 (250 mg, 1.091 mmol) was added to the mixture. The mixture was stirred at rt for 1 h under N₂. The mixture was quenched with aq. NH₄Cl solution (15 mL) and extracted with EtOAc (2×15 mL). The combined organic layers were dried over Na₂SO₄, filtered, and concentrated under reduced pressure to give crude product. The residue was purified by prep-TLC separation to give 1-9, which was checked by LCMS: MS m/z (ESI) [M+H+2]⁺=422.0.

To a solution of 1-15 (10 g, 64.94 mmol) in DCM (200 mL) at room temperature under N₂ was added CDI (15.78 g, 97.44 mmol). The reaction mixture was stirred for 0.5 h. Then Et₃N (19.68 g, 194.82 mmol) and N,O-dimethylhydroxylamine hydrogen chloride (7.6 g, 77.93 mmol) were added to the above solution. The reaction mixture was stirred at room temperature for 48 h. The mixture was diluted with DCM (100 mL) and washed with 1 M HCl (3×300 mL), saturated NaHCO₃ solution (300 mL). The organic layer was dried over anhydrous Na₂SO₄, filtered and concentrated under reduced pressure to give 1-16, which was used for the next step without further purification.

To a mixture of 1-16 (5 g, 25.36 mmol) in dioxane (50 mL) was added LiAlH₄ (25.36 mL, 1 M) at 0° C. under N₂. The mixture was stirred at 0-20° C. for 2 h. The mixture was quenched with H₂O (1 mL), 15% NaOH (1 mL) and H₂O (31 mL). The mixture was purified by distillation at 95-97° C. under standard atmospheric pressure to give 1-17, which was used for the next step without further purification.

To a mixture of 1-17 (10 g, 15.21 mmol) and K₂CO₃ (6.31 g, 45.62 mmol) in MeOH (25 mL) was added dimethyl (1-diazo-2-oxopropyl)phosphonate (5.84 g, 30.42 mmol) at 0° C. under N₂. The mixture was stirred at 20° C. for 18 h. The mixture was diluted with toluene (50 mL) and washed with H₂O (3×50 mL). The mixture was purified by distillation under standard atmospheric pressure to give 1-10, which was used for the next step without further purification.

To a mixture of 1-9 (260 mg, 0.309 mmol) and 1-10 (112.00 mg, 0.309 mmol) in DMF (0.5 mL) was added ZnBr₂ (347.94 mg, 1.545 mmol), Pd(dppf)Cl₂ (22.61 mg, 0.031 mmol) and TEA (0.859 mL, 6.180 mmol) under N₂. The mixture was stirred at 100° C. for 15 min in a sealed tube. The mixture was diluted with EtOAc (20 mL). The organic layer was washed with brine (3×20 mL), dried over Na₂SO₄, filtered, and concentrated under reduced pressure to give crude product. The crude product was purified by silica gel column chromatography to give the crude product.

80 mg of the crude product was further purified by prep-HPLC separation to give example 1. LCMS: MS m/z (ESI) [M+H]⁺=474.1; ¹H NMR (400 MHz, DMSO) δ 7.99 (d, J=9.0 Hz, 1H), 7.60 (d, J=8.9 Hz, 1H), 7.31-7.21 (m, 1H), 7.14 (t, J=7.8 Hz, 1H), 6.88 (d, J=8.0 Hz, 1H), 6.18 (s, 1H), 4.90 (s, 2H), 4.15-3.95 (m, 2H), 3.83-3.75 (m, 2H), 3.54 (s, 3H), 1.54-1.34 (m, 4H).

### Examples 2: 5-methyl-6-oxo-8-(6-((1-(trifluoromethyl)cyclopropyl)ethynyl)-2,3-dihydrobenzo[e][1,4]oxazepin-1(5H)-yl)-5,6-dihydro-1,5-naphthyridine-2-carbonitrile

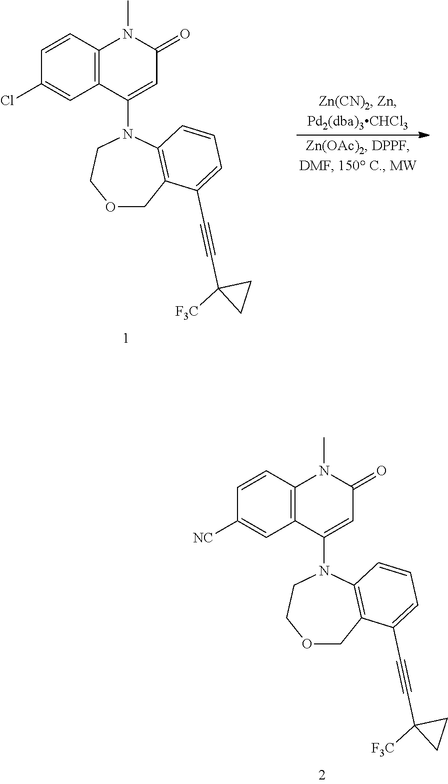 [SMILES: Cn1c(=O)cc(N2CCOCc3c(C#CC4(C(F)(F)F)CC4)cccc32)c2cc(Cl)ccc21 ; Cn1c(=O)cc(N2CCOCc3c(C#CC4(C(F)(F)F)CC4)cccc32)c2cc(C#N)ccc21]

To a mixture of compound 1 (100 mg, 0.211 mmol) in DMF (2 mL) was added Pd₂(dba)₃·CHCl₃ (21.84 mg, 0.021 mmol), Zn(CN)₂ (49.56 mg, 0.422 mmol), Zn (13.80 mg, 0.211 mmol), zinc bis(acetate) (3.87 mg, 0.021 mmol) and DPPF (23.82 mg, 0.042 mmol). The mixture was stirred at 150° C. for 1 h under microwave in a sealed tube. The mixture was diluted with EtOAc (20 mL). The organic layer was washed with brine (3×20 mL), dried over Na₂SO₄, filtered, and concentrated under reduced pressure to give crude product. The crude product was purified by silica gel column chromatography and prep-TLC separation to give example 2. LCMS: MS m/z (ESI) [M+H]⁺=465.0; ¹H NMR (400 MHz, DMSO) δ 8.07 (s, 2H), 7.29 (d, J=7.6 Hz, 1H), 7.14 (t, J=7.8 Hz, 1H), 6.89 (d, J=8.1 Hz, 1H), 6.26 (s, 1H), 4.91 (s, 2H), 4.15-4.00 (m, 2H), 3.85-3.75 (m, 2H), 3.56 (s, 3H), 1.50-1.41 (m, 4H).

### Examples 3: 6-chloro-1-methyl-4-(6-((1-(trifluoromethyl)cyclopropyl)ethynyl)-2,3-dihydrobenzo[e][1,4]oxazepin-1(5H)-yl)quinolin-2(1H)-one

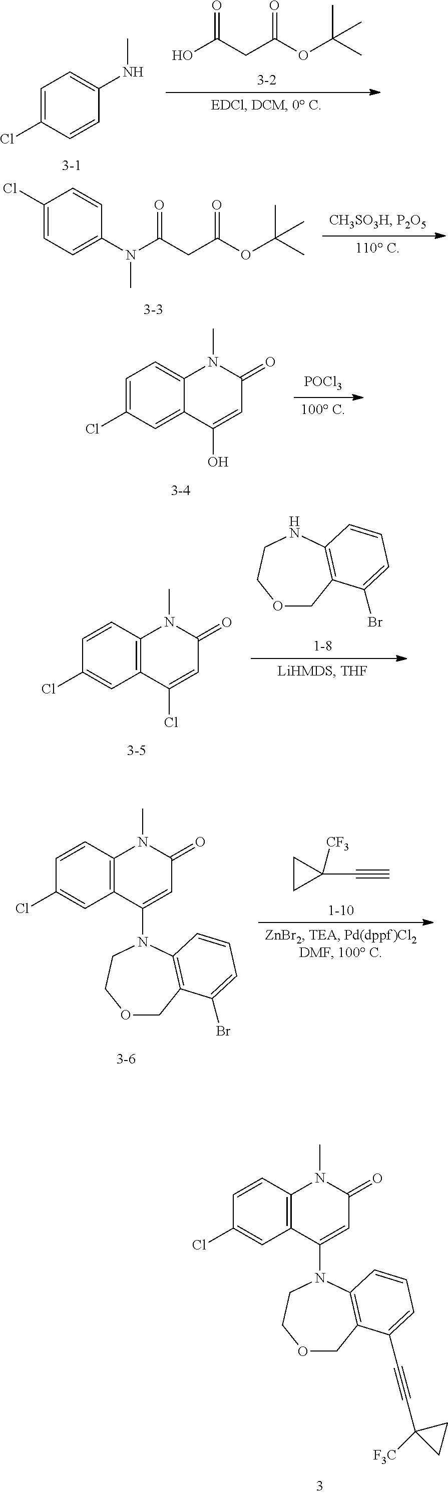

To a solution of 3-1 (5 g, 35.31 mmol) in DCM (200 mL) was added 3-2 (5.77 g, 36.02 mmol) at 0 C, then EDCI (6.90 g, 36.02 mmol) was added in separated batches. The reaction mixture was stirred at 0° C. for 1 h. The mixture was washed with 1 M NaOH (50 mL×3), then washed with 1 M HCl (50 mL×3). The organic layer was dried over Na₂SO₄, filtered, and concentrated under reduced pressure to give 3-3, which was checked by LCMS: MS m/z (ESI) [M+H−57]⁺=228.1.

To a solution of 3-3 (10 g, 32.72 mmol) in methanesulfonic acid (50 mL) was added phosphorus pentoxide (6.97 g, 49.08 mmol). The reaction mixture was stirred at 60° C. for 0.5 h and then at 110° C. for 0.5 h. The mixture was cooled to room temperature and poured into ice-water (150 mL). The mixture was stirred for 0.5 h and then filtered. The filter cake was dried under reduced pressure to give 3-4, which was checked by LCMS: MS m/z (ESI) [M+H+CH₃CN]⁺=251.1.

A mixture of 3-4 (2 g, 9.143 mmol) in POCl₃ (30 mL) was stirred at 100° C. for 3 h. The mixture was evaporated and poured into ice-water (20 mL). The mixture was adjusted to pH=8.0 with saturated sodium bicarbonate solution and extracted with DCM (100 mL×3). The combined organic layers were dried over Na₂SO₄, filtered, and concentrated under reduced pressure to give 3-5, which was checked by LCMS: MS m/z (ESI) [M+H]⁺=228.1.

To a mixture of 1-8 (150 mg, 0.658 mmol) in THF (5 mL) was added LiHMDS (1.973 mL) at 20° C. under N₂. The mixture was stirred at 20° C. for 0.5 h under N₂. Then 3-5 (180.08 mg, 0.790 mmol) was added to the mixture. The mixture was stirred at 20° C. for 1 h under N₂. The mixture was quenched with aq. NH₄Cl solution (15 mL) and extracted with EtOAc (2×15 mL). The combined organic layers were dried over Na₂SO₄, filtered, and concentrated under reduced pressure to give crude product. The residue was purified by column chromatography on silica gel to give 3-6, which was checked by LCMS: MS m/z (ESI) [M+H+2]⁺=420.8.

To a mixture of 3-6 (170 mg, 0.405 mmol) and 1-10 (146.80 mg, 0.405 mmol) in DMF (1.5 mL) was added ZnBr₂ (456.07 mg, 2.025 mmol), Pd(dppf)Cl₂ (29.64 mg, 0.041 mmol) and TEA (1.126 mL, 8.101 mmol) under N₂. The mixture was stirred at 100° C. for 15 min in a sealed tube. The mixture was diluted with EtOAc (20 mL). The organic layer was washed with brine (3×20 mL), dried over Na₂SO₄, filtered, and concentrated under reduced pressure to give crude product. The residue was purified by column chromatography on silica gel to give the crude product. 30 mg of the crude product was purified by prep-HPLC separation to give example 3. MS m/z (ESI) [M+H]⁺=473.2; ¹H NMR (400 MHz, DMSO) δ 7.56 (s, 2H), 7.20 (d, J=7.5 Hz, 1H), 7.11-6.98 (m, 2H), 6.66-6.56 (m, 2H), 5.05 (s, 2H), 3.93-3.83 (m, 2H), 3.80-3.62 (m, 2H), 3.615 (s, 3H), 1.53-1.43 (m, 4H).

### Example 4: 1-methyl-2-oxo-4-(6-((1-(trifluoromethyl)cyclopropyl)ethynyl)-2,3-dihydrobenzo[e][1,4]oxazepin-1(5H)-yl)-1,2-dihydroquinoline-6-carbonitrile

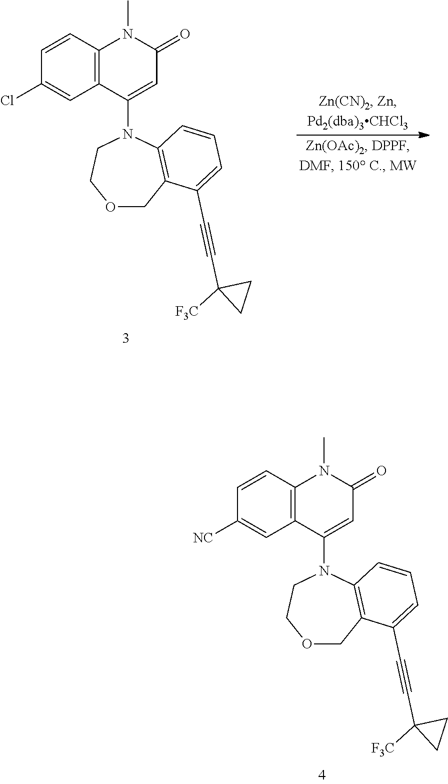 [SMILES: Cn1c(=O)cc(N2CCOCc3c(C#CC4(C(F)(F)F)CC4)cccc32)c2cc(Cl)ccc21 ; Cn1c(=O)cc(N2CCOCc3c(C#CC4(C(F)(F)F)CC4)cccc32)c2cc(C#N)ccc21]

To a mixture of compound 3 (60 mg, 0.127 mmol) in DMA (2 mL) was added Pd₂(dba)₃·CHCl₃ (13.13 mg, 0.013 mmol), Zn(CN)₂ (29.80 mg, 0.254 mmol), Zn (8.30 mg, 0.127 mmol), zinc bis(acetate) (2.33 mg, 0.013 mmol) and DPPF (14.32 mg, 0.025 mmol). The mixture was stirred at 150° C. for 1 h under microwave in a sealed tube. The mixture was filtered and the filter cake was washed with MeOH (10 mL). The filtrate was concentrated under reduced pressure. The residue was purified by prep-HPLC separation to give example 4. MS m/z (ESI) [M+H]⁺ 464.2; ¹H NMR (400 MHz, DMSO) δ 7.91 (dd, J=8.8, 1.8 Hz, 1H), 7.69 (d, J=8.9 Hz, 1H), 7.48 (d, J=1.8 Hz, 1H), 7.21 (d, J=6.9 Hz, 1H), 7.05 (t, J=7.9 Hz, 1H), 6.70-6.56 (m, 2H), 5.10 (s, 2H), 3.93-3.83 (m, 2H), 3.80-3.66 (m, 2H), 3.64 (s, 3H), 1.53-1.51 (m, 4H).

### Example 5: 6-chloro-1-methyl-4-(6-((1-(trifluoromethyl)cyclopropyl)ethynyl)-2,3-dihydrobenzo[e][1,4]oxazepin-1(5H)-yl)quinazolin-2(1H)-one

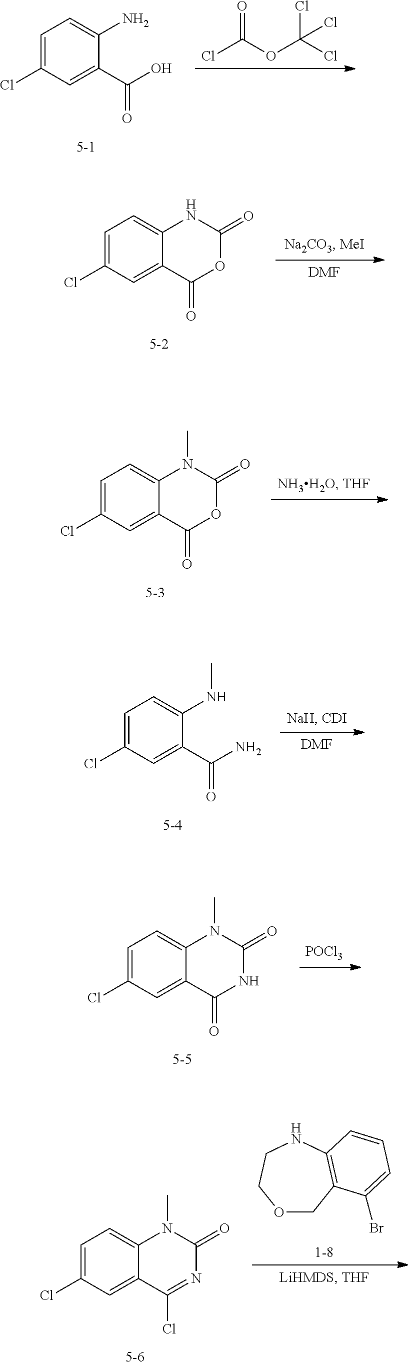

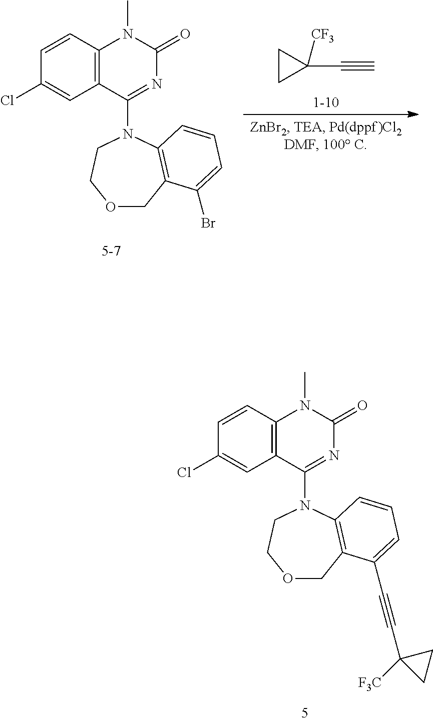

To a mixture of 5-1 (10 g, 58.282 mmol) in dioxane (100 mL) was added trichloromethyl chloromethanoate (86.47 g, 437.114 mmol). The reaction mixture was stirred at 110° C. for 4 h. The reaction mixture was filtered and the filter cake was dried under reduced pressure to give 5-2. ¹H NMR (400 MHz, DMSO) δ 11.86 (s, 1H), 7.87 (d, J=0.8 Hz, 1H), 7.78 (dd, J=8.8, 2.3 Hz, 1H), 7.16 (d, J=8.8 Hz, 1H).

To a mixture of 5-2 (10 g, 50.615 mmol) in DMF (100 mL) was added Na₂CO₃ (6.44 g, 60.738 mmol) and CH₃I (5 mL, 75.912 mmol). The reaction mixture was stirred at rt for 12 h under N₂. The mixture was quenched with water (300 mL). The reaction mixture was filtered and the filter cake was dried under reduced pressure to give 5-3. ¹H NMR (400 MHz, DMSO) δ 7.96 (d, J=1.6 Hz, 1H), 7.93-7.87 (m, 1H), 7.48 (d, J=8.8 Hz, 1H), 3.46 (s, 3H).

To a mixture of 5-3 (1.5 g, 7.09 mmol) in THE (10 mL) was added ammonium hydroxide (0.546 mL, 14.18 mmol). The mixture was stirred at 60° C. for 2 h. The reaction mixture was diluted with ethyl acetate (30 mL). The organic layer was washed with water (30 mL) and sat. NaCl (2×30 mL), dried over Na₂SO₄, filtered, and concentrated under reduced pressure to give 5-4. LCMS: MS m/z (ESI) [M+H]⁺ 185.2; 1H NMR (400 MHz, DMSO) δ 7.92 (s, 1H), 7.65 (d, J=2.4 Hz, 1H), 7.30 (dd, J=8.9, 2.4 Hz, 1H), 6.64 (d, J=9.2 Hz, 1H), 2.77 (s, 3H).

To a mixture of 5-4 (1.00 g, 5.42 mmol) in DMF (15 mL) was added NaH (1.08 g, 27.08 mmol, 60% in mineral oil) at 0° C. under N₂. The reaction mixture was warmed to room temperature and stirred for 1 h under N₂. CDI (1.32 g, 8.13 mmol) was added to the mixture. The mixture was stirred at 70° C. for 2 h under N₂. The mixture was diluted with DCM (15 ml), adjusted to pH=4-5 with 1 M HCl and precipitate formed. The mixture was filtered, and the filter cake was washed with H₂O (20 mL). The filter cake was dried under reduced pressure to give product 5-5. ¹H NMR (400 MHz, DMSO) δ 11.70 (s, 1H), 7.92 (t, J=7.6 Hz, 1H), 7.80 (dd, J=8.8, 2.4 Hz, 1H), 7.46 (d, J=9.2 Hz, 1H), 3.43 (s, 3H).

To a mixture of 5-5 (100 mg, 0.475 mmol) in toluene (5 mL) was added POCl₃ (0.221 mL, 2.374 mmol) and DIEA (0.392 mL, 2.374 mmol). The reaction mixture was stirred at 110° C. for 3 h. The mixture was concentrated under reduced pressure to give 5-6, which was used for the next step without further purification.

To a mixture of 1-8 (99.58 mg, 0.437 mmol) in THF (5 mL) was added LiHMDS (1.310 mL) at 20° C. under N₂. The mixture was stirred for 0.5 h. Then 5-6 (100 mg, 0.437 mmol) was added to the mixture. The mixture was stirred at 20° C. for 1 h under N₂. The mixture was quenched with aq. NH₄Cl solution (15 mL) and extracted with EtOAc (2×15 mL). The combined organic layers were dried over Na₂SO₄, filtered, and concentrated under reduced pressure. The resulting residue was purified by prep-TLC separation to give 5-7, which was checked by LCMS: MS m/z (ESI) [M+H]⁺ 420.1.

To a mixture of 5-7 (34 mg, 0.081 mmol) and 1-10 (29.29 mg, 0.081 mmol) in DMF (0.2 mL) was added ZnBr₂ (91.00 mg, 0.404 mmol), Pd(dppf)Cl₂ (5.91 mg, 0.008 mmol) and TEA (0.225 mL, 1.616 mmol) under N₂. The mixture was stirred at 100° C. for 15 min in a sealed tube. The mixture was diluted with EtOAc (20 mL). The organic layer was washed with brine (3×20 mL), dried over Na₂SO₄, filtered, and concentrated under reduced pressure. The resulting residue was purified by prep-HPLC separation to give example 5. LCMS: MS m/z (ESI) [M+H]⁺ 474.1; ¹H NMR (400 MHz, DMSO) δ 7.63 (dd, J=9.1, 2.3 Hz, 1H), 7.50-7.43 (m, 2H), 7.24 (t, J=7.9 Hz, 1H), 7.04 (d, J=7.5 Hz, 1H), 6.57 (d, J=2.3 Hz, 1H), 5.05 (s, 2H), 4.04-3.90 (m, 4H), 3.52 (s, 3H), 1.58-1.40 (m, 4H).

### Example 6: 1-methyl-2-oxo-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

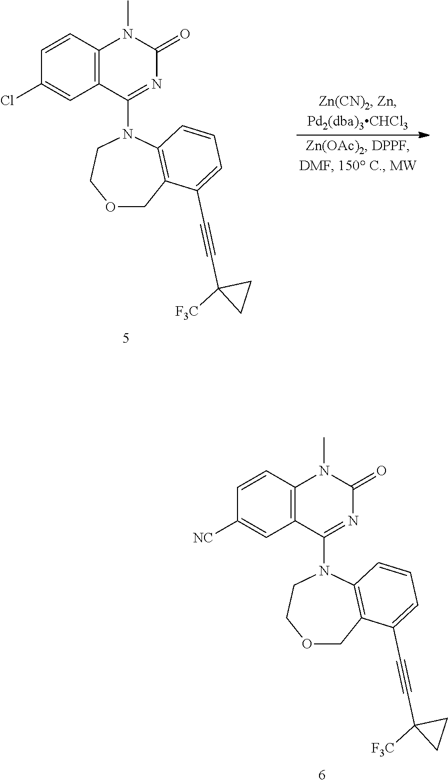 [SMILES: Cn1c(=O)nc(N2CCOCc3c(C#CC4(C(F)(F)F)CC4)cccc32)c2cc(Cl)ccc21 ; Cn1c(=O)nc(N2CCOCc3c(C#CC4(C(F)(F)F)CC4)cccc32)c2cc(C#N)ccc21]

To a mixture of compound 5 (30 mg, 0.063 mmol) in DMA (2 mL) was added Pd₂(dba)₃·CHCl₃ (6.55 mg, 0.006 mmol), Zn(CN)₂ (14.87 mg, 0.127 mmol), Zn (4.14 mg, 0.063 mmol), zinc bis(acetate) (1.16 mg, 0.006 mmol) and DPPF (7.15 mg, 0.013 mmol). The mixture was stirred at 150° C. for 1 h under microwave in a sealed tube. The mixture was diluted with EtOAc (20 mL). The organic layer was washed with brine (3×20 mL), dried over Na₂SO₄, filtered, and concentrated under reduced pressure to give crude product. The residue was purified by prep-HPLC separation to give example 6. LCMS: MS m/z (ESI) [M+H]⁺ 465.2; ¹H NMR (400 MHz, DMSO) δ 7.95 (dd, J=8.9, 1.7 Hz, 1H), 7.57 (d, J=9.0 Hz, 1H), 7.49 (d, J=7.6 Hz, 1H), 7.23 (t, J=7.9 Hz, 1H), 7.05 (d, J=7.9 Hz, 1H), 6.97 (d, J=1.7 Hz, 11H), 5.05 (s, 2H), 4.10-3.80 (m, 2H), 3.54 (s, 3H), 3.40-3.33 (m, 2H), 1.60-1.40 (m, 4H).

### Example 7: 6-chloro-1-methyl-4-(6-((1-(trifluoromethyl)cyclopropyl)ethynyl)-2,3-dihydrobenzol[e][1,4]oxazepin-1(5H)-yl)pyrido[3,2-d]pyrimidin-2(1H)-one

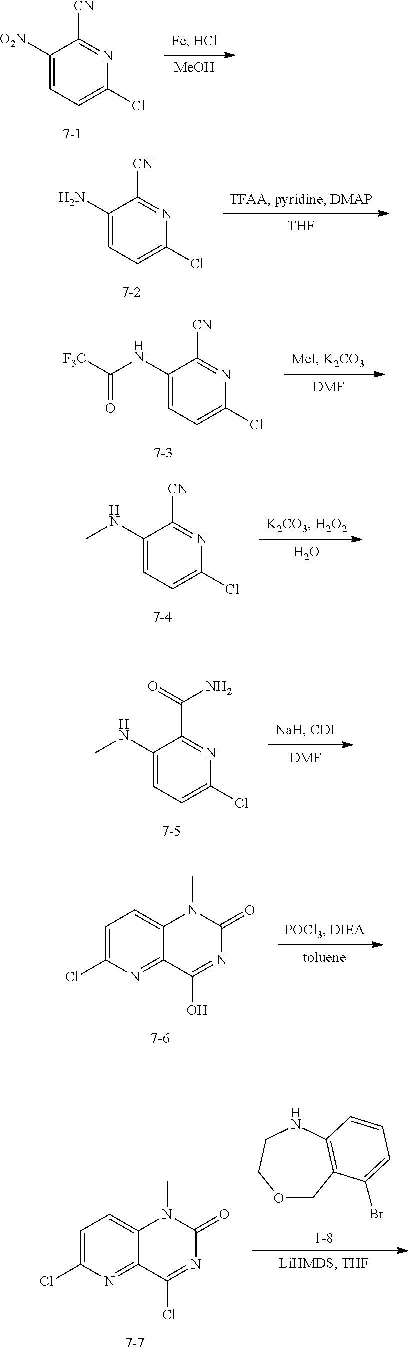

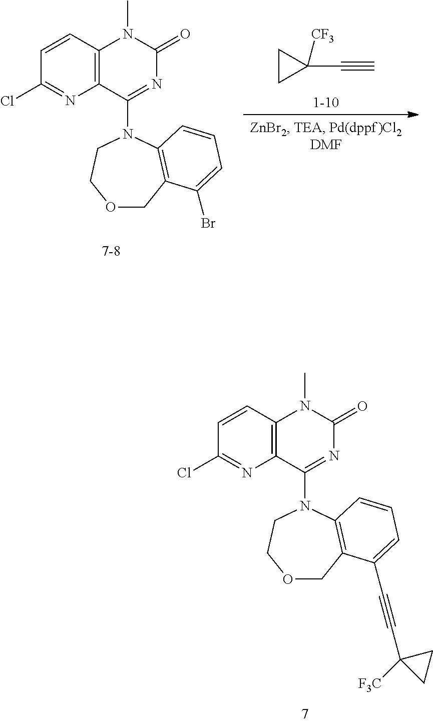

To a mixture of 7-1 (5.0 g, 27.2 mmol) in MeOH (60 mL) and HCl (25 mL, 37% w/w) was added Fe (1.07 g, 19.1 mmol) at 20° C. After stirring at 80° C. for 1 h, the reaction mixture was cooled to room temperature and poured into ice-water (100 mL). The mixture was filtered and the filter cake was washed with DCM (100 mL). The filtrate was extracted with DCM (100 mL×2). The combine organic layers were washed with brine (300 mL), dried over Na₂SO₄, filtered, and concentrated under reduced pressure to give 7-2, which was directly used in the next step without further purification.

To a suspension of 7-2 (3.3 g, 21.5 mmol) in THF (50 mL) were added DMAP (1.31 g, 10.75 mmol), pyridine (6.84 mL, 86 mmol) and TFAA (5.50 mL, 43 mmol). The mixture was stirred at 25° C. for 2 h. The reaction mixture was filtered and the filtrate was evaporated in reduced pressure. The resulting residue was dissolved in DCM (80 mL) and the mixture was washed with water (80 mL). The organic layer was dried over Na₂SO₄, filtered, and concentrated under reduced pressure to afford 7-3, which was directly used for the next step without further purification.

To a solution of 7-3 (2.5 g, 10.0 mmol) in DMF (15 mL) was added Mel (2.63 g, 18.5 mmol) and K₂CO₃ (4.15 g, 30.1 mmol). After stirring at 20° C. for 16 h, the reaction mixture was quenched with water (50 mL) and extracted with ethyl acetate (50 mL×3). The combined organic layers were dried over Na₂SO₄, filtered, and concentrated under reduced pressure to afford 7-4, which was checked by LCMS: MS m/z (ESI) [M+H]⁺=168.2. To a solution of 7-4 (1.4 g, 8.35 mmol) in DMSO (20 mL) and H₂O (5.0 mL) was added K₂CO₃ (2.31 g, 16.71 mmol) and H₂O₂(1.89 g, 16.71 mmol). After stirring at 20° C. for 16 h, the reaction mixture was diluted with water (40 mL) and the precipitate formed. The mixture was filtered and the filter cake was dried under reduced pressure to give 7-5.

To a solution of 7-5 (580 mg, 3.13 mmol) in DMF (5 mL) was added NaH (375 mg, 9.37 mmol, 60% in mineral oil) at 0° C. under N₂. After stirring at room temperature for 1 h, a solution of CDI (760.04 mg, 4.687 mmol) in DMF (3 mL) was added to the above solution. The reaction mixture was allowed to warm to 70° C. and stirred for another 2 h. After cooling to room temperature, the precipitate formed and the mixture was filtered. The filter cake was washed with water and dried under reduced pressure to afford 7-6, which was checked by LCMS: MS m/z (ESI) [M+H]⁺=212.0.

To a suspension of 7-6 (200 mg, 0.95 mmol) in dry toluene (8 mL) were added POCl₃ (0.44 mL, 4.73 mmol) and DIEA (0.78 mL, 4.73 mmol) at room temperature. The reaction mixture was heated at 110° C. for 2 h under N₂. The reaction mixture was concentrated under reduced pressure to give 7-7, which was directly used for next step without further purification.

To a mixture of 1-8 (130 mg, 0.57 mmol) in THF (5 mL) was added LiHMDS (1.71 mL) at 20° C. under N₂. After stirring at 20° C. for 5 min, DIEA (0.94 mL, 5.70 mmol) and a solution of 7-7 (150 mg, 0.65 mmol) in THE (3 mL) were added to the mixture at −10° C. After stirring at −10° C. for 1 h under N₂, the mixture was quenched with saturated NH₄Cl solution (15 mL) and extracted with DCM (15 mL×2). The combined organic layers were dried over Na₂SO₄, filtered, and concentrated under reduced pressure. The resulting residue was purified by column chromatography on silica gel to give 7-8, which was checked by LCMS: MS m/z (ESI) [M+H]⁺=422.9.

To a mixture of 7-8 (120 mg, 0.18 mmol) and 1-10 (98.13 mg, 0.18 mmol) in DMF (0.5 mL) was added ZnBr₂ (202 mg, 0.90 mmol), Pd(dppf)Cl₂ (13.1 mg, 0.018 mmol) and TEA (0.50 mL, 3.58 mmol) under N₂. After stirring at 100° C. for 15 min, the mixture was diluted with EtOAc (20 mL). The mixture was washed with brine (20 mL×3), dried over Na₂SO₄, filtered, and concentrated under reduced pressure. The resulting residue was purified by prep-HPLC separation to give example 7. LCMS: MS m/z (ESI) [M+H]⁺ 475.1. ¹H NMR (400 MHz, DMSO) δ 7.91 (d, J=9.0 Hz, 1H), 7.65 (d, J=9.0 Hz, 1H), 7.41 (d, J=7.7 Hz, 1H), 7.22 (t, J=7.8 Hz, 1H), 7.14 (d, J=7.5 Hz, 1H), 5.50-4.35 (m, 3H), 4.22-3.75 (m, 2H), 3.48 (s, 3H), 3.46-3.25 (m, 1H), 1.55-1.35 (m, 4H).

### Example 8: 2-oxo-4-(6-((1-(trifluoromethyl)cyclopropyl)ethynyl)-2,3-dihydrobenzo[e][1,4]oxazepin-1(5H)-yl)-1,2-dihydropyrido[3,2-d]pyrimidine-6-carbonitrile

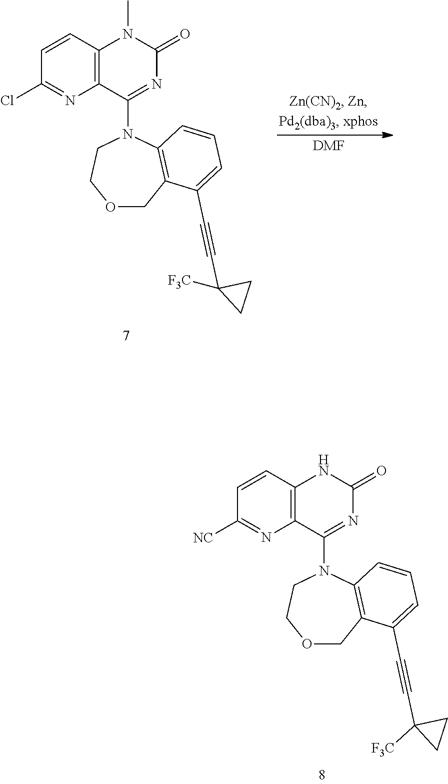

To a mixture of compound 7 (250 mg, 0.53 mmol) in DMF (5 mL) was added Pd₂(dba)₃ (48.2 mg, 0.053 mmol), Zn(CN)₂ (309 mg, 2.63 mmol), Zn (34.4 mg, 0.53 mmol) and XPHOS (50.2 mg, 0.11 mmol). The mixture was stirred at 120° C. for 1 h under microwave in a sealed tube. The mixture was diluted with EtOAc (20 mL) and the organic layer was washed with brine (20 mL×3), dried over Na₂SO₄, filtered, and concentrated under reduced pressure. The resulting residue was purified by prep-HPLC separation to give example 8. LCMS: MS m/z (ESI) [M+H]⁺ 466.2; ¹H NMR (400 MHz, DMSO) δ 8.09 (d, J=8.5 Hz, 1H), 7.99 (d, J=9.0 Hz, 1H), 7.43 (d, J=7.5 Hz, 1H), 7.27-7.11 (m, 2H), 5.57-4.38 (m, 3H), 4.15-3.77 (m, 2H), 3.50 (s, 3H), 3.40-2.96 (m, 1H), 1.53-1.39 (m, 4H).

### Example 9: 6-fluoro-1-methyl-4-(6-((1-(trifluoromethyl)cyclopropyl)ethynyl)-2,3-dihydrobenzo[e][1,4]oxazepin-1(5H)-yl)quinazolin-2(1H)-one

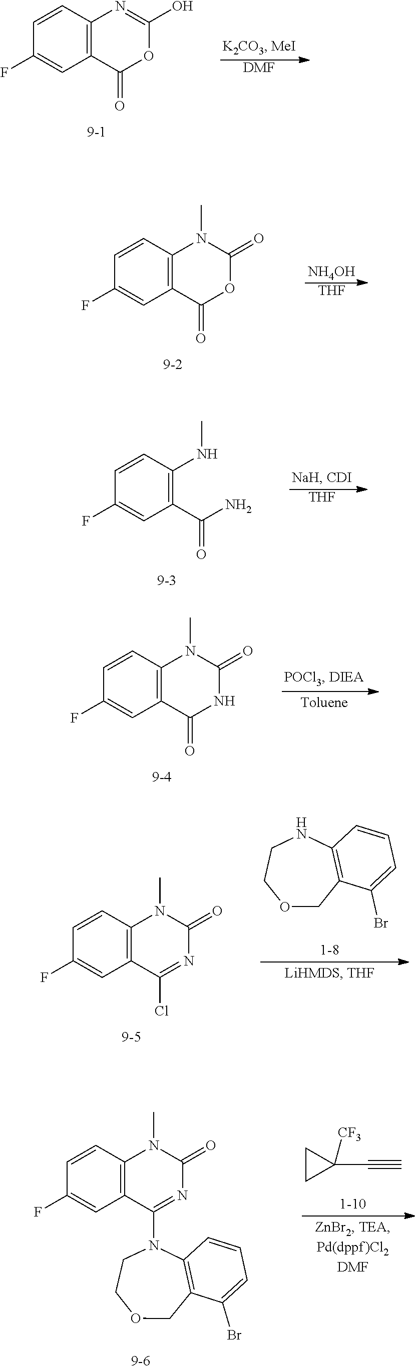

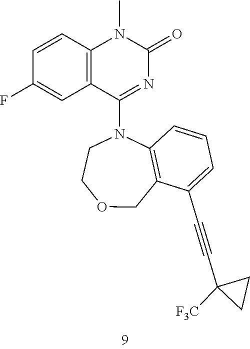 [SMILES: Cn1c(=O)nc(N2CCOCc3c(C#CC4(C(F)(F)F)CC4)cccc32)c2cc(F)ccc21]

To a mixture of compound 9-1 (3 g, 16.56 mmol) in DMF (20 mL) was added K₂CO₃ (6.87 g, 49.69 mmol) and Mel (3.53 g, 24.85 mmol). The mixture was stirred at 20° C. for 2 h under N₂. The mixture was quenched with H₂O (50 mL) and precipitate formed. The mixture was filtered. The filter cake was washed with H₂O (20 mL) and dried under reduced pressure to give 9-2, which was used for the next step without further purification. ¹H NMR (400 MHz, DMSO) δ 7.85-7.68 (m, 2H), 7.58-7.41 (m, 1H), 3.47 (s, 3H).

To a mixture of compound 9-2 (600 mg, 3.08 mmol) in THF (7 mL) was added ammonium hydroxide (3.5 mL, 25.44 mmol). The mixture was stirred at 15° C. for 30 min. The mixture was concentrated under reduced pressure. A solution of HCl (4 M) in MeOH was added to the residue and the mixture was stirred at 15° C. for 16 h. The mixture was concentrated under reduced pressure. The residue was adjusted to pH=8-9 with aq. NaHCO₃ solution and extracted with DCM (50 mL). The organic layer was washed with brine (50 mL×3), dried over Na₂SO₄, filtered, and concentrated under reduced pressure to give compound 9-3, which was used for the next step without further purification. MS (ESI) m/z (M+H)⁺=169.0.

To a mixture of compound 9-3 (440 mg, 2.62 mmol) in THF (5 mL) was added NaH (313.92 mg, 13.08 mmol, 60% in mineral oil) and CDI (636.28 mg, 3.92 mmol). The mixture was stirred at 15° C. for 3 h under N₂. The mixture was quenched with sat. NH₄Cl solution (30 mL) and the precipitate formed. The mixture was filtered. The filter cake was rinsed with DCM (30 mL) and dried under reduced pressure to give compound 9-4. MS (ESI) m/z (M+H)⁺=195.0.

To a mixture of compound 9-4 (356 mg, 1.83 mmol) in toluene (5 mL) was added DIEA (1.52 mL, 9.17 mmol) and POCl₃ (0.85 mL, 9.17 mmol). The mixture was stirred at 110° C. for 3 h under N₂. The residue was adjusted to pH-7-8 with aq. NaHCO₃ solution and extracted with DCM (30 mL). The organic layer was washed with brine (3×30 mL), dried over Na₂SO₄, filtered, and concentrated under reduced pressure to give compound 9-5, which was used for the next step without further purification. MS (ESI) m/z (M+H)⁺=212.9.

To a mixture of 1-8 in THE (8 mL) was added LiHMDS (2.37 mL) at 20° C. under N₂. After stirring for 10 min at 20° C., DIEA (1.30 mL, 7.89 mmol) and 9-5 (252 mg, 1.18 mmol) were added to the mixture at −10° C. The mixture was stirred at −10° C. for 1 h under N₂. The mixture was quenched with aq. NH₄Cl solution (15 mL) and extracted with EtOAc (15 mL×2). The combined organic layers were dried over Na₂SO₄, filtered, and concentrated under reduced pressure. The resulting residue was purified by column chromatography on silica gel to give 9-6. MS (ESI) m/z (M+H)⁺=404.0.

To a mixture of 9-6 (170 mg, 0.25 mmol) and 1-10 (166 mg, 0.30 mmol) in DMF (1 mL) was added ZnBr₂ (284 mg, 1.26 mmol), Pd(dppf)Cl₂ (18.5 mg, 0.025 mmol) and TEA (0.70 mL, 5.05 mmol) under N₂. The mixture was stirred at 100° C. for 15 min in a sealed tube. The mixture was diluted with EtOAc (20 mL). The organic layer was washed with brine (20 mL×3), dried over Na₂SO₄, filtered, and concentrated under reduced pressure. The resulting residue was purified by prep-HPLC separation to give example 9. MS (ESI) m/z (M+H)⁺=458.2. ¹H NMR (400 MHz, DMSO) δ 7.60-7.40 (m, 3H), 7.22 (t, J=7.9 Hz, 1H), 7.01 (d, J=7.9 Hz, 1H), 6.34 (dd, J=10.2, 2.7 Hz, 1H), 5.25-4.25 (m, 4H), 4.03-3.80 (m, 2H), 3.54 (s, 3H), 1.56-1.40 (m, 4H).

### Example 10: 6-fluoro-1-methyl-2-oxo-4-(6-((1-(trifluoromethyl)cyclopropyl)ethynyl)-2,3-dihydrobenzo[e][1,4]oxazepin-1(5H)-yl)-1,2-dihydroquinoline-3-carbonitrile

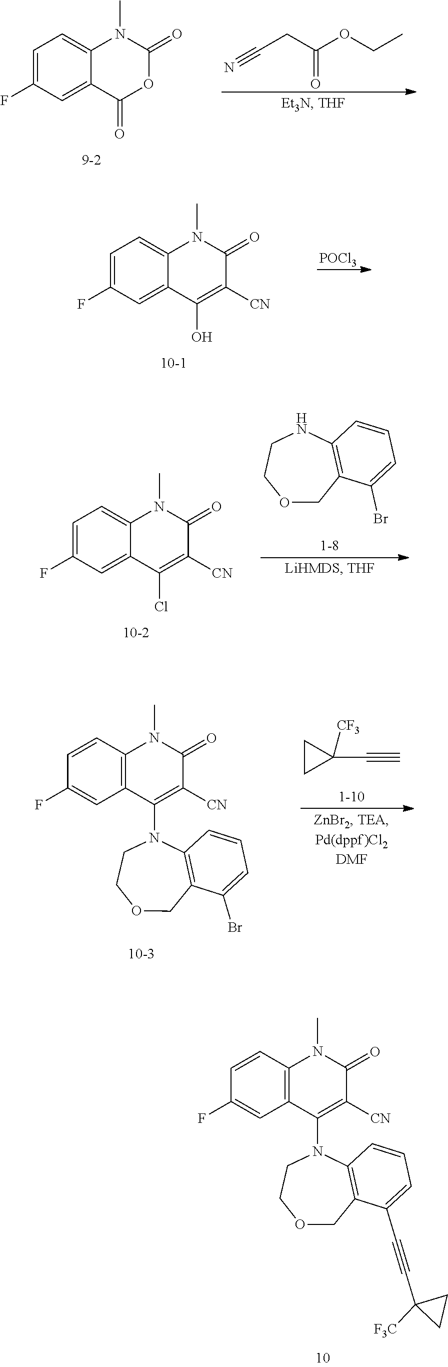

To a mixture of 9-2 (100 mg, 0.51 mmol) in THF (5 mL) was added ethyl 2-cyanoacetate (0.295 mL, 2.77 mmol) and Et₃N (0.71 mL, 5.12 mmol). The reaction mixture was stirred at 90° C. for 72 h. The reaction was diluted with EtOAc (20 mL) and H₂O (10 mL). The organic layer was separated, and then washed with brine, dried over Na₂SO₄, filtered, and concentrated under reduced pressure to give 10-1. MS (ESI) m/z (M+H)⁺=218.9. ¹H NMR (400 MHz, DMSO) δ 7.82 (dd, J=9.3, 2.9 Hz, 1H), 7.71-7.51 (m, 2H), 3.54 (s, 3H).

A mixture of 10-1 (500 mg, 2.29 mmol) in POCl₃ (2.5 mL) was stirred at 90° C. for 12 h. After cooling to room temperature, the reaction mixture was diluted with hexane and the resulting solid was collected by filtration. The filter cake was carefully added to saturated sodium bicarbonate solution. The solid was filtered and dried under reduced pressure to give 10-2. ¹H NMR (400 MHz, CDCl₃) δ 7.80 (dd, J=8.6, 2.8 Hz, 1H), 7.53 (tt, J=20.0, 10.0 Hz, 1H), 7.44 (dd, J=9.3, 4.2 Hz, 1H), 3.76 (s, 3H).

To a mixture of 1-8 (48.2 mg, 0.21 mmol) in THF (3 mL) was added LiHMDS (0.63 mL) at room temperature under N₂. After stirring for 10 min, 10-2 (50 mg, 0.21 mmol) was added to the mixture at −10° C. under N₂. The reaction mixture was stirred at −10° C. for 1 h. The mixture was quenched with aq. NH₄Cl solution (15 mL) and extracted with EtOAc (15 mL×2). The combined organic layers were dried over Na₂SO₄, filtered, and concentrated under reduced pressure to give 10-3, which was directly used for the next step without further purification. MS (ESI) m/z (M+H)⁺=427.9.

To a mixture of 10-3 (110 mg, 0.26 mmol) and 1-10 (141 mg, 0.26 mmol) in DMF (0.5 mL) were added ZnBr₂ (289 mg, 1.28 mmol), Pd(dppf)Cl₂ (18.8 mg, 0.026 mmol) and TEA (0.71 mL, 5.14 mmol) under N₂. The mixture was stirred at 100° C. for 15 min in a sealed tube. The mixture was diluted with EtOAc (20 mL). The organic layer was washed with brine (20 mL×3), dried over Na₂SO₄, filtered, and concentrated under reduced pressure. The resulting residue was purified by prep-HPLC separation to give example 10. MS (ESI) m/z (M+H)⁺=482.1. ¹H NMR (400 MHz, DMSO) δ 7.75-7.60 (m, 2H), 7.34 (dd, J=9.5, 2.6 Hz, 1H), 7.20 (d, J=7.4 Hz, 1H), 7.09 (t, J=7.9 Hz, 1H), 6.79 (d, J=8.1 Hz, 1H), 5.17 (d, J=14.4 Hz, 1H), 5.04 (d, J=14.5 Hz, 1H), 4.17-4.07 (m, 1H), 4.06-3.80 (m, 3H), 3.66 (s, 3H), 1.56-1.40 (m, 4H).

### Example 11: 6-chloro-1-methyl-7-((tetrahydrofuran-3-yl)oxy)-4-(6-((1-(trifluoromethyl)cyclopropyl)ethynyl)-2,3-dihydrobenzo[e][1,4]oxazepin-1(5H)-yl)quinazolin-2(1H)-one

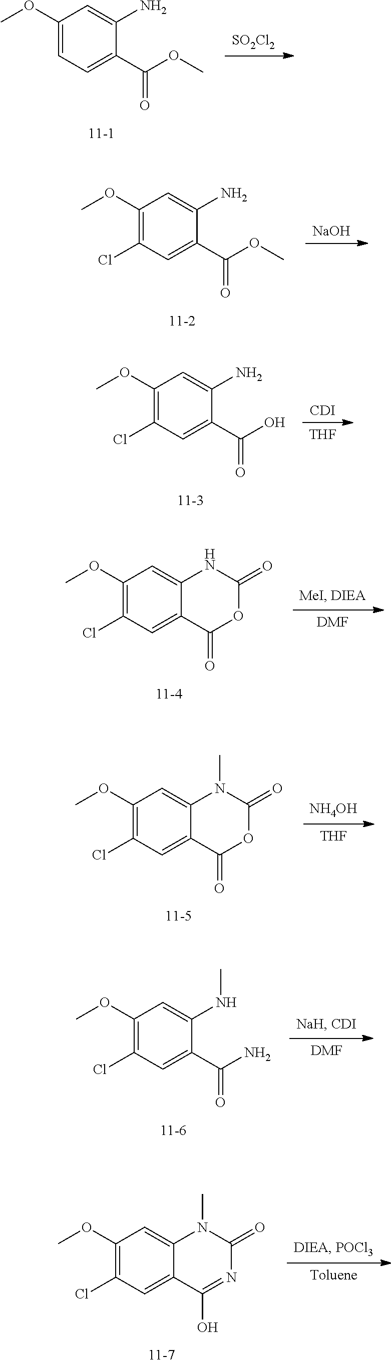

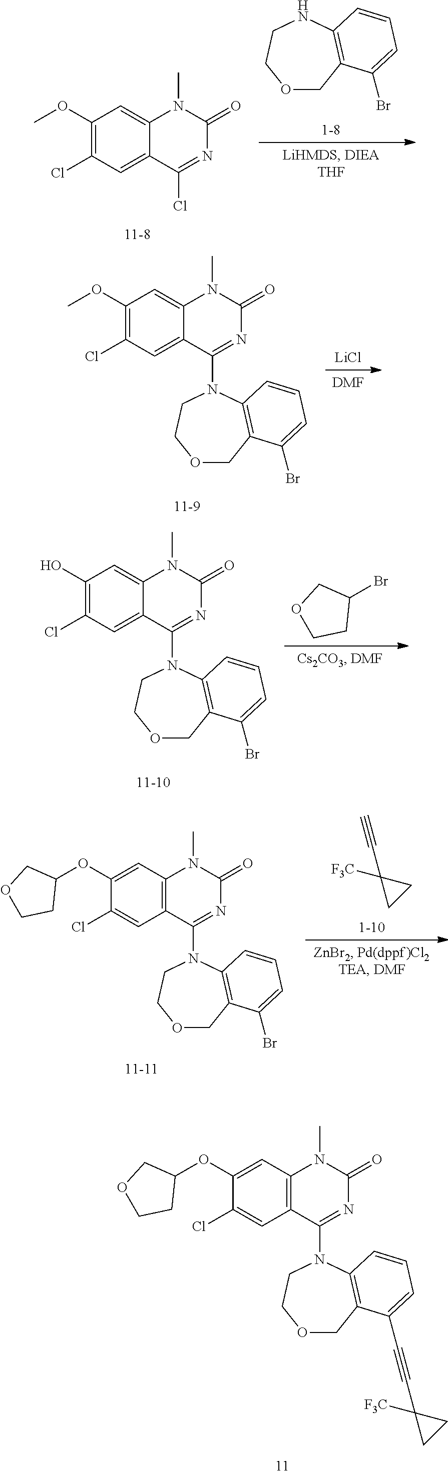

To a solution of 11-1 (10.0 g, 55.2 mmol) in CHCl₃ (100 mL) was added SO₂Cl₂ (8.94 g, 66.23 mmol) at ice bath temperature. The mixture was refluxed for 12 h. The reaction mixture was concentrated under reduced pressure to give 11-2. MS (ESI) m/z (M+H)⁺=216.1. To a solution of NaOH (2.77 g, 69.3 mmol) in H₂O (100 mL) and THF (100 mL) was added 11-2 (9.0 g, 34.64 mmol). The mixture was stirred at 105° C. overnight. The reaction mixture was acidified with HCl (2M) and the precipitate formed. The precipitate was collected by filtration. The filter cake was washed with water and dried under reduced pressure to provide 11-3, MS (ESI) m/z (M+MeCN+H)⁺=243.1. A mixture of 11-3 (6.0 g, 29.8 mmol) and CDI (4.83 g, 29.76 mmol) in THF (100 mL) was stirred at room temperature for 12 h. The mixture was quenched with water and precipitate formed. The precipitate was collected by filtration, and the filter cake was washed with water and dried under reduced pressure to give 11-4. MS (ESI) m/z (M−H)⁺=226.0.

To a mixture of 11-4 (4.2 g, 18.5 mmol) in DMF (50 mL) was added DIEA (6.10 mL, 36.9 mmol) and CH₃I (7.86 g, 55.4 mmol). The reaction mixture was stirred at rt for 12 h. The reaction was quenched with ice and the precipitate formed. The precipitate was collected by filtration and the filter cake was washed with water and dried under reduced pressure to give 11-5 as a brown solid. MS (ESI) m/z (M+H)⁺=242.1.

To a mixture of 11-5 (3.0 g, 12.4 mmol) in THF (20 mL) was added NH₄OH (0.96 mL, 24.8 mmol). After stirring at 60° C. for 2 h, the reaction mixture was diluted with EtOAc (80 mL) and washed with water and brine. The organic layer was collected, dried over Na₂SO₄, filtered, and concentrated in vacuo to give 11-6. MS (ESI) m/z (M+H)⁺=214.9.

To a mixture of 11-6 (2.0 g, 9.32 mmol) in DMF (50 mL) was added NaH (1.86 g, 46.6 mmol, 60% in mineral oil) at 0° C. After stirring at room temperature for 1 h, the mixture was added CDI (2.27 g, 13.98 mmol). The reaction mixture was stirred at 70° C. for 2 h. After cooling to room temperature, the mixture was diluted with water (30 mL) and filtered. The filter cake was washed with 1 M HCl solution and dried under reduced pressure to 11-7. MS (ESI) m/z (M+H)⁺=241.1.

To a suspension of 11-7 (1.0 g, 4.16 mmol) in dry toluene (30 mL) were added POCl₃ (1.93 mL, 20.8 mmol) and DIEA (3.43 mL, 20.8 mmol) at room temperature. The reaction mixture was heated at 110° C. for 2 h under N₂. The reaction mixture was concentrated under reduced pressure to give 11-8, which was directly used for next step without further purification.

To a mixture of 1-8 (1.42 g, 6.23 mmol) in THF (30 mL) was added LiHMDS (18.7 mL) at 20° C. under N₂. The mixture was stirred at 20° C. for 5 min under N₂. Then DIEA (6.86 mL, 41.53 mmol) and a solution of 11-8 (1.08 g, 4.15 mmol) in THF (10 mL) were added to the mixture. The mixture was stirred at 20° C. for 1 h under N₂. The mixture was quenched with aq. NH₄Cl solution (50 mL) and extracted with DCM (50 mL×2). The combined organic layers were dried over Na₂SO₄, filtered, and concentrated under reduced pressure. The resulting residue was purified by column chromatography on silica gel to give 11-9. MS (ESI) m/z (M+H)⁺=450.0.

To a mixture of 11-9 (200 mg, 0.44 mmol) in DMF (1 mL) was added LiCl (113 mg, 2.66 mmol). The mixture was stirred at 140° C. for 18 h. The mixture was concentrated under reduced pressure. The residue was diluted with DCM/MeOH (10/1, 15 mL) and filtered. The filtrate was concentrated under reduced pressure to give 11-10, which was directly used for the next step without further purification. MS (ESI) m/z (M+H)⁺=436.0.

To a mixture of 11-10 (110 mg, 0.25 mmol) in DMF (3 mL) was added Cs₂CO₃ (410 mg, 1.26 mmol) and 3-bromotetrahydrofuran (114 mg, 0.76 mmol). The mixture was stirred at 100° C. for 4 h under N₂. The mixture was diluted with EtOAc (30 mL). The organic layer was washed with brine (30 mL×3), dried over Na₂SO₄, filtered, and concentrated under reduced pressure. The resulting residue was purified by column chromatography on silica gel to give 11-11. MS (ESI) m/z (M+H)⁺=506.0.

To a mixture of 11-11 (63 mg, 0.124 mmol) and 1-10 (68.0 mg, 0.124 mmol) in DMF (0.8 mL) was added ZnBr₂ (140 mg, 0.622 mmol), Pd(dppf)Cl₂ (9.10 mg, 0.012 mmol) and Et₃N (0.35 mL, 2.49 mmol) under N₂. The mixture was stirred at 100° C. for 15 min in a sealed tube. The mixture was diluted with EtOAc (20 mL). The organic layer was washed with brine (20 mL×3), dried over Na₂SO₄, filtered, and concentrated under reduced pressure. The resulting residue was purified by prep-HPLC separation to give example 11. MS (ESI) m/z (M+H)⁺=560.2. ¹H NMR (400 MHz, DMSO) δ 7.46 (d, J=7.8 Hz, 1H), 7.24 (t, J=7.9 Hz, 1H), 7.02 (d, J=7.4 Hz, 1H), 6.86 (s, 1H), 6.57 (s, 1H), 5.45-5.33 (m, 1H), 5.15-4.75 (m, 2H), 4.13-3.68 (m, 6H), 3.55 (s, 3H), 3.40-3.25 (m, 2H), 2.40-2.24 (m, 1H), 2.06-1.91 (m, 1H), 1.55-1.40 (m, 4H).

### Example 12: 1-methyl-2-oxo-7-((tetrahydrofuran-3-yl)oxy)-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

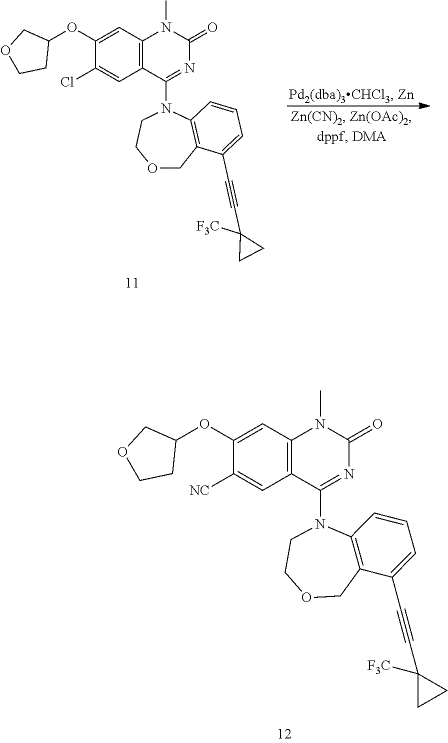 [SMILES: Cn1c(=O)nc(N2CCOCc3c(C#CC4(C(F)(F)F)CC4)cccc32)c2cc(Cl)c(OC3CCOC3)cc21 ; Cn1c(=O)nc(N2CCOCc3c(C#CC4(C(F)(F)F)CC4)cccc32)c2cc(C#N)c(OC3CCOC3)cc21]

To a mixture of compound 11 (90 mg, 0.153 mmol) in DMA (3 mL) was added Zn(CN)₂ (89.6 mg, 0.76 mmol), Zn (10 mg, 0.15 mmol), zinc bis(acetate) (28 mg, 0.15 mmol), dppf (25.9 mg, 0.046 mmol) and Pd₂(dba)₃CHCl₃ (31.6 mg, 0.031 mmol). The mixture was stirred at 150° C. for 2 h under microwave in a sealed tube. The mixture was filtered and the filtrate was purified by pre-HPLC separation to give example 12. MS (ESI) m/z (M+H)⁺=551.3. ^{f}H NMR (400 MHz, DMSO) δ 7.48 (d, J=7.7 Hz, 1H), 7.24 (t, J=7.9 Hz, 1H), 7.04 (d, J=7.9 Hz, 1H), 6.87 (s, 2H), 5.47-5.42 (m, 1H), 5.16-4.82 (m, 2H), 3.97-3.74 (m, 6H), 3.56 (s, 3H), 3.40-3.25 (m, 2H), 2.38-2.29 (m, 1H), 2.05-1.96 (m, 1H), 1.53-1.46 (m, 4H).

### Example 13: 6-chloro-7-(2-hydroxyethoxy)-1-methyl-4-(6-((1-(trifluoromethyl)cyclopropyl)ethynyl)-2,3-dihydrobenzo[e][1,4]oxazepin-1(5H)-yl)quinazolin-2(1H)-one

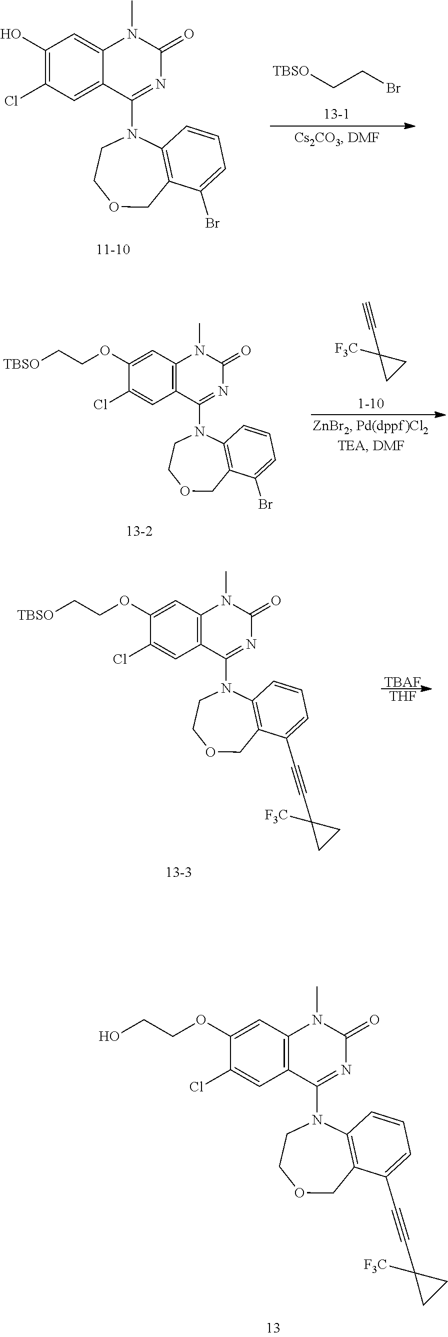

To a mixture of 11-10 (120 mg, 0.28 mmol) and 13-1 (197 mg, 0.82 mmol) in DMF (5 mL) was added Cs₂CO₃ (228 mg, 1.65 mmol). The reaction was stirred at 60° C. for 16 h. The reaction mixture was diluted with EtOAc (10 ml) and washed with water and brine. The organic layer was dried over Na₂SO₄, filtered, and concentrated in vacuum to give 13-2. MS (ESI) m/z (M+H)⁺=594.2.

To a mixture of 13-2 (110 mg, 0.19 mmol) in DMF (1 mL) was added ZnBr₂ (208 mg, 0.93 mmol), Pd(dppf)Cl₂ (13.5 mg, 0.019 mmol), TEA (0.51 mL, 3.70 mmol) and 1-10 (121 mg, 0.22 mmol). The reaction mixture was stirred at 100° C. for 15 min in a sealed tube. The reaction mixture was diluted with EtOAc (30 mL) and washed with brine. The organic layer was dried over Na₂SO₄, filtered, and concentrated in vacuum. The resulting residue was purified by column chromatography on silica gel to give example 13-3. MS (ESI) m/z (M+H)⁺=648.3.

To a mixture of 13-3 (40 mg, 0.063 mmol) in THF (3 mL) was added TBAF (0.12 mL, 0.12 mmol). The reaction mixture was stirred at room temperature for 2 h. The mixture was concentrated under reduced pressure. The resulting residue was purified by prep-HPLC separation to give example 13. MS (ESI) m/z (M+H)⁺=534.1. ¹H NMR (400 MHz, DMSO) δ 7.46 (d, J=7.7 Hz, 1H), 7.24 (t, J=7.9 Hz, 1H), 7.00 (d, J=7.9 Hz, 1H), 6.94 (s, 1H), 6.57 (s, 1H), 5.25-4.75 (m, 3H), 4.25 (t, J=4.6 Hz, 2H), 3.99-3.86 (m, 2H), 3.80-3.74 (m, 2H), 3.56 (s, 3H), 3.50-3.24 (m, 2H), 1.54-1.46 (m, 4H).

### Example 14: 7-(2-hydroxyethoxy)-1-methyl-2-oxo-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

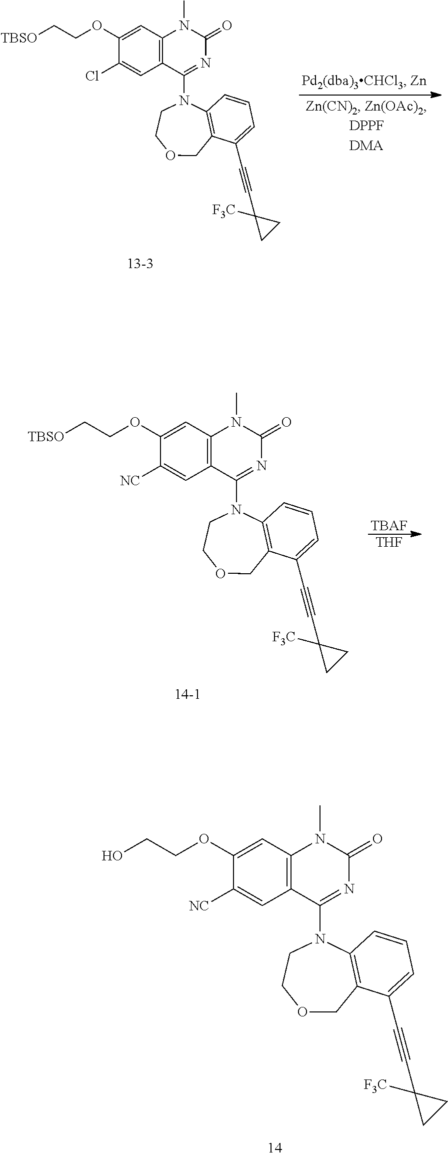

To a mixture of 13-3 (70 mg, 0.11 mmol), Zn (7.06 mg, 0.11 mmol), Zn(OAc)₂ (2.0 mg, 0.011 mmol), Zn(CN)₂ (25.4 mg, 0.22 mmol) and DPPF (12.0 mg, 0.022 mmol) in DMA (3 mL) was added Pd₂(dba)₃CHCl₃ (12 mg, 0.011 mmol). The reaction was stirred at 150° C. under microwave for 2 h in a sealed tube. The reaction mixture was diluted with EtOAc (30 ml) and washed with brine. The organic layer was dried over Na₂SO₄, filtered, and concentrated in vacuum. The resulting residue was purified by silica gel column chromatography to give 14-1. MS (ESI) m/z (M+H)⁺=639.4.

To a mixture of 14-1 (60 mg, 0.094 mmol) in THE (5 mL) was added TBAF (0.094 mL, 0.188 mmol, 1 M in THF). The reaction mixture was stirred at room temperature for 2 h. The mixture was concentrated under reduced pressure. The resulting residue was purified by prep-HPLC separation to give example 14. MS (ESI) m/z (M+H)⁺=525.2. ¹H NMR (400 MHz, DMSO) δ 7.48 (d, J=7.6 Hz, 1H), 7.24 (t, J=8.0 Hz, 1H), 7.03 (d, J=8.0 Hz, 1H), 6.94 (s, 1H), 6.86 (s, 1H), 5.20-4.90 (m, 3H), 4.31 (t, J=4.4 Hz, 2H), 4.17-3.81 (m, 2H), 3.79-3.69 (m, 2H), 3.55 (s, 3H), 3.34-3.25 (m, 2H), 1.55-1.45 (m, 4H).

### Example 15: 6-chloro-7-((2-hydroxyethyl)amino)-1-methyl-4-(6-((1-(trifluoromethyl)cyclopropyl)ethynyl)-2,3-dihydrobenzo[e][1,4]oxazepin-1(5H)-yl)quinazolin-2(1H)-one

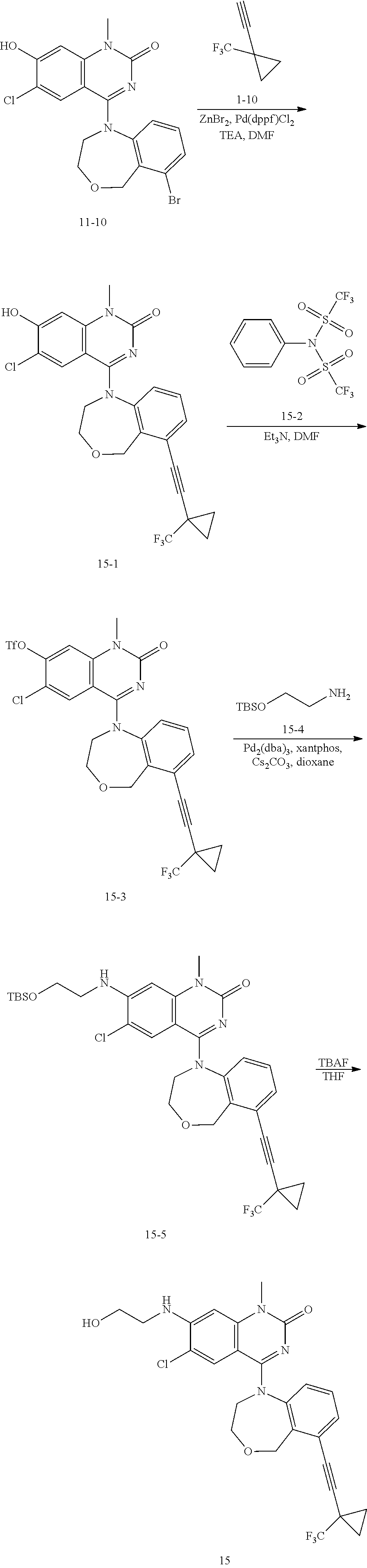

To a mixture of 11-10 (1.0 g, 2.29 mmol) and 1-10 (1.57 g, 1.15 mmol) in DMF (4 mL) was added ZnBr₂ (1.55 g, 6.87 mmol), Pd(dppf)Cl₂ (0.17 g, 0.23 mmol) and Et₃N (3.82 mL, 27.48 mmol) under N₂. The mixture was stirred at 100° C. for 15 min in a sealed tube. The mixture was concentrated under reduced pressure. The resulting residue was purified by column chromatography on silica gel to give 15-1. MS (ESI) m/z (M+H)⁺=490.1.

To a solution of 15-1 (50 mg, 0.097 mmol) in DMF (10 mL) was added Et₃N (0.081 mL, 0.58 mmol) and 15-2 (52.0 mg, 0.15 mmol). The mixture was stirred at 25° C. for 18 h under N₂. The mixture was poured into water (100 mL) and extracted with DCM (50 mL×3). The combined organic layers were dried over Na₂SO₄, filtered, and concentrated under reduced pressure. The resulting residue was purified by flash column chromatography to give 15-3. MS (ESI) m/z (M+H)⁺=622.0.

To a solution of 15-3 (40 mg, 0.061 mmol) in dioxane (10 mL) was added 15-4 (32.14 mg, 0.183 mmol), Pd₂(dba)₃ (5.60 mg, 0.006 mmol), xantphos (3.54 mg, 0.006 mmol) and Cs₂CO₃ (99.5 mg, 0.31 mmol). The mixture was stirred at 100° C. for 5 h under N₂. The mixture was poured into water (100 mL) and extracted with DCM (50 mL×3). The combined organic layers were dried over Na₂SO₄, filtered, and concentrated under reduced pressure. The resulting residue was purified by flash column chromatography on silica gel to give 15-5. MS (ESI) m/z (M+H)⁺=647.2.

To a solution of 15-5 (30 mg, 0.042 mmol) in THF (3 mL) was added TBAF solution (0.21 mL, 0.21 mmol). The mixture was stirred at 25° C. for 18 h under N₂. The mixture was poured into water (15 mL) and extracted with DCM (10 mL×3). The combined organic layers were dried over Na₂SO₄, filtered, and concentrated under reduced pressure. The resulting residue was purified by preparative HPLC separation to give example 15. MS (ESI) m/z (M+H)⁺=533.0. ¹H NMR (400 MHz, DMSO) δ 7.42 (d, J=7.7 Hz, 1H), 7.22 (t, J=7.8 Hz, 1H), 6.96 (d, J=8.0 Hz, 1H), 6.45 (s, 1H), 6.38 (s, 1H), 6.16 (t, J=5.4 Hz, 1H), 5.23-4.70 (m, 3H), 3.96-3.80 (m, 2H), 3.64-3.56 (m, 2H), 3.49 (s, 3H), 3.28-3.42 (m, 4H), 1.52-1.46 (m, 4H).

### Example 16: 7-((2-hydroxyethyl)amino)-1-methyl-2-oxo-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

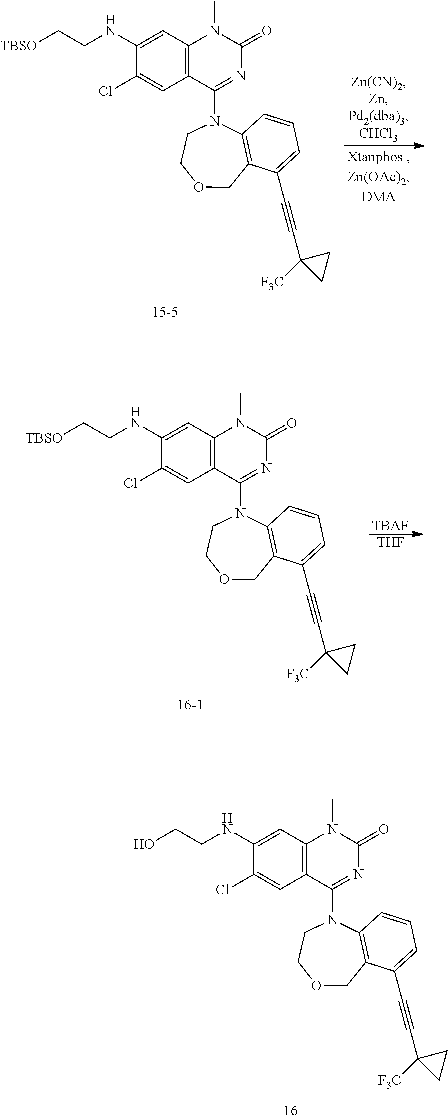

To a solution of 15-5 (60 mg, 0.088 mmol) in DMA (1 mL) was added Zn(CN)₂ (51.7 mg, 0.44 mmol), Zn (5.8 mg, 0.088 mmol), Zn(OAc)₂ (17.0 mg, 0.093 mmol), xantphos (26.8 mg, 0.046 mmol) and Pd₂(dba)₃CHCl₃ (26.8 mg, 0.046 mmol). The mixture was stirred at 150° C. for 1 h under microwave in a sealed tube. The mixture was poured into water (100 mL) and extracted with DCM (50 mL×3). The combined organic layers were dried over Na₂SO₄, filtered, and concentrated under reduced pressure. The resulting residue was purified by flash column chromatography on silica gel to give 16-1. MS (ESI) m/z (M+H)⁺=638.3.

To a solution of 16-1 (30 mg, 0.041 mmol) in THF (1 mL) was added TBAF solution (0.12 mL, 1 M). The mixture was stirred at 25° C. for 16 h under N₂. The mixture was poured into water (20 mL) and extracted with DCM (15 mL×3). The combined organic layers were dried over Na₂SO₄, filtered, and concentrated under reduced pressure. The resulting residue was purified by preparative HPLC separation to give example 16. MS (ESI) m/z (M+H)⁺=524.2. ¹H NMR (400 MHz, DMSO) δ 7.45 (dd, J=7.7, 1.0 Hz, 1H), 7.24 (t, J=7.9 Hz, 1H), 7.06-6.97 (m, 1H), 6.68 (s, 1H), 6.60 (t, J=5.6 Hz, 1H), 6.35 (s, 1H), 5.25-4.65 (m, 3H), 4.25-3.70 (m, 2H), 3.64-3.54 (m, 2H), 3.47 (s, 3H), 3.42-3.25 (m, 4H), 1.53-1.44 (m, 4H).

### Example 17: 6-chloro-7-(2-methoxyethoxy)-1-methyl-4-(6-((1-(trifluoromethyl)cyclopropyl)ethynyl)-2,3-dihydrobenzo[e][1,4]oxazepin-1(5H)-yl)quinazolin-2(1H)-one

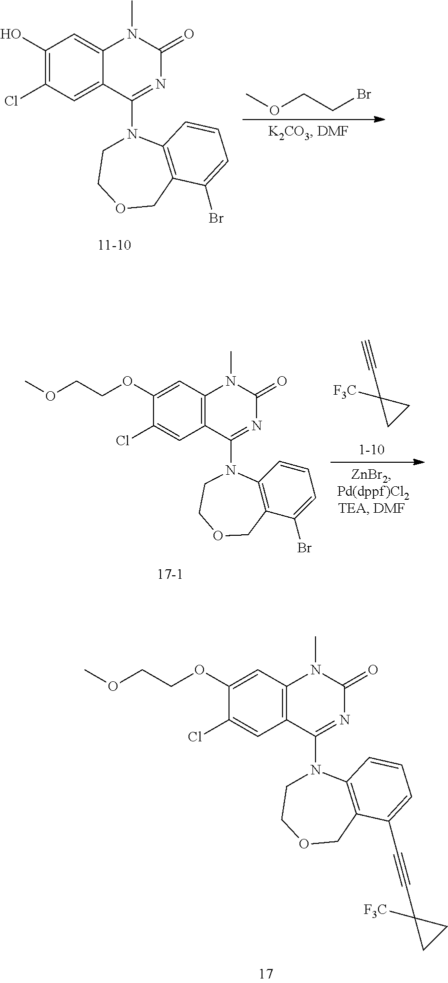

To a mixture of 11-10 (180 mg, 0.41 mmol) in DMF (2 mL) was added K₂CO₃ (285 mg, 2.06 mmol) and 1-bromo-2-methoxyethane (172 mg, 1.24 mmol). The mixture was stirred at 50° C. for 18 h under N₂. The mixture was diluted with EtOAc (30 mL) and washed with brine (30 mL×3), dried over Na₂SO₄, filtered, and concentrated under reduced pressure. The resulting residue was purified by column chromatography on silica gel to give 17-1. MS (ESI) m/z (M+H)⁺=494.0.

To a mixture of 17-1 (100 mg, 0.20 mmol) and 1-10 (111 mg, 0.20 mmol) in DMF (2 mL) was added ZnBr₂ (227 mg, 1.01 mmol), Pd(dppf)Cl₂ (14.8 mg, 0.020 mmol) and Et₃N (0.56 mL, 4.04 mmol) under N₂. The mixture was stirred at 100° C. for 15 min in a sealed tube. The mixture was diluted with EtOAc (20 mL), washed with brine (20 mL×3), dried over Na₂SO₄, filtered, and concentrated under reduced pressure. The resulting residue was purified by prep-HPLC separation to give example 17. MS (ESI) m/z (M+H)⁺=548.2. ¹H NMR (400 MHz, DMSO) δ 7.45 (d, J=7.6 Hz, 1H), 7.23 (t, J=7.9 Hz, 1H), 7.00 (d, J=7.9 Hz, 1H), 6.93 (s, 1H), 6.56 (s, 1H), 5.22-4.73 (m, 2H), 4.45-4.25 (m, 2H), 4.03-3.83 (m, 2H), 3.76-3.66 (m, 2H), 3.55 (s, 3H), 3.34-3.31 (m, 5H), 1.57-1.40 (m, 4H).

### Example 18: 7-(2-methoxyethoxy)-1-methyl-2-oxo-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

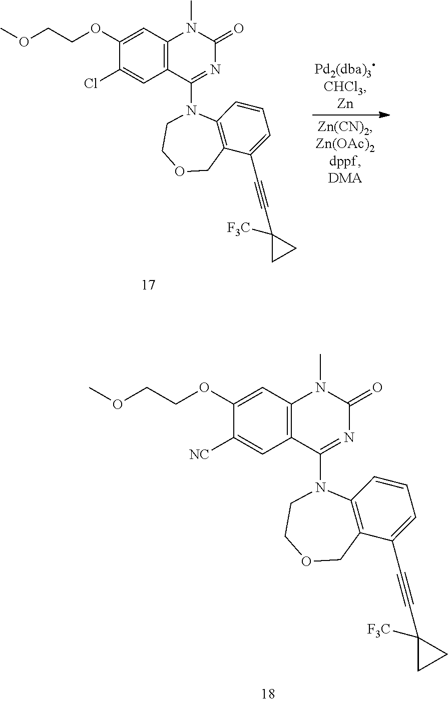 [SMILES: COCCOc1cc2c(cc1Cl)c(N1CCOCc3c(C#CC4(C(F)(F)F)CC4)cccc31)nc(=O)n2C ; COCCOc1cc2c(cc1C#N)c(N1CCOCc3c(C#CC4(C(F)(F)F)CC4)cccc31)nc(=O)n2C]

To a mixture of compound 17 (60 mg, 0.11 mmol) in DMA (1 mL) was added Pd₂(dba)₃-CHCl₃ (11.3 mg, 0.011 mmol), Zn(CN)₂ (25.7 mg, 0.22 mmol), Zn (7.2 mg, 0.11 mmol), Zn(OAc)₂ (2.0 mg, 0.011 mmol) and dppf (12.4 mg, 0.022 mmol). The mixture was stirred at 150° C. for 2 h under microwave in a sealed tube. The mixture was filtered and the filter cake was washed with MeOH (10 mL). The filtrate was concentrated under reduced pressure. The resulting residue was purified by prep-HPLC separation to give example 18. MS (ESI) m/z (M+H)⁺=539.3. ¹H NMR (400 MHz, DMSO) δ 7.48 (d, J=7.5 Hz, 1H), 7.23 (t, J=7.9 Hz, 1H), 7.03 (d, J=7.9 Hz, 1H), 6.94 (s, 1H), 6.86 (s, 1H), 5.16-4.90 (m, 2H), 4.45-4.36 (m, 2H), 4.02-3.83 (m, 2H), 3.74-3.68 (m, 2H), 3.55 (s, 3H), 3.32 (s, 3H), 3.29-3.28 (m, 2H), 1.53-1.45 (m, 4H).

### Example 19: 6-chloro-1-methyl-7-morpholino-4-(6-((1-(trifluoromethyl)cyclopropyl)ethynyl)-2,3-dihydrobenzo[e][1,4]oxazepin-1(5H)-yl)quinazolin-2(1H)-one

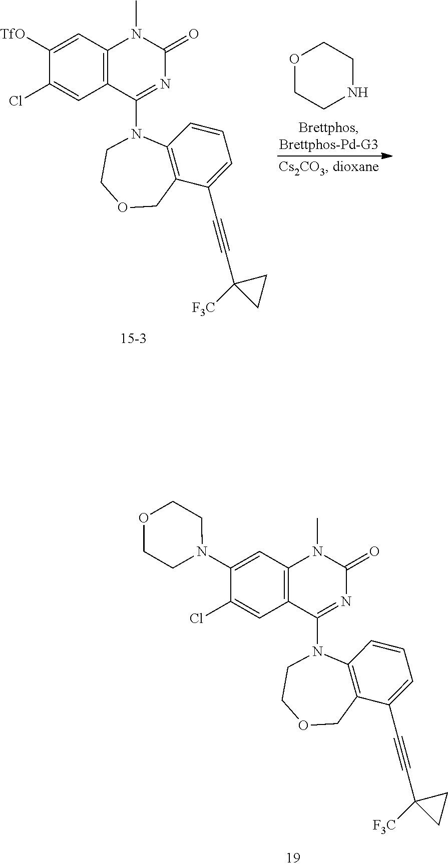 [SMILES: Cn1c(=O)nc(N2CCOCc3c(C#CC4(C(F)(F)F)CC4)cccc32)c2cc(Cl)c(OS(=O)(=O)C(F)(F)F)cc21 ; C1COCCN1 ; Cn1c(=O)nc(N2CCOCc3c(C#CC4(C(F)(F)F)CC4)cccc32)c2cc(Cl)c(N3CCOCC3)cc21]

To a solution of 15-3 (100 mg, 0.15 mmol) in dioxane (10 mL) was added morpholine (0.050 mL, 0.76 mmol), Brettphos-Pd-G3 (42 mg, 0.046 mmol), Brettphos (25 mg, 0.046 mmol) and Cs₂CO₃ (248.8 mg, 0.76 mmol). The mixture was stirred at 100° C. for 5 h under N₂. The mixture was poured into water (20 mL) and extracted with DCM (30 mL×3). The combined organic layers were dried over Na₂SO₄, filtered, and concentrated under reduced pressure. The resulting residue was purified by preparative HPLC separation to give example 19. MS (ESI) m/z (M+H)⁺=559.2. ¹H NMR (400 MHz, CD₃OD) δ 7.51 (d, J=7.6 Hz, 1H), 7.28 (t, J=7.8 Hz, 1H), 7.00 (d, J=7.7 Hz, 1H), 6.91 (s, 1H), 6.71 (s, 1H), 5.26-4.75 (m, 2H), 4.12-3.95 (m, 2H), 3.92-3.78 (m, 4H), 3.69 (s, 3H), 3.40-3.14 (m, 6H), 1.54-1.39 (m, 4H).

### Example 20: 7-(3-aminopropoxy)-6-chloro-1-methyl-4-(6-((1-(trifluoromethyl)cyclopropyl)ethynyl)-2,3-dihydrobenzo[e][1,4]oxazepin-1(5H)-yl)quinazolin-2(1H)-one H

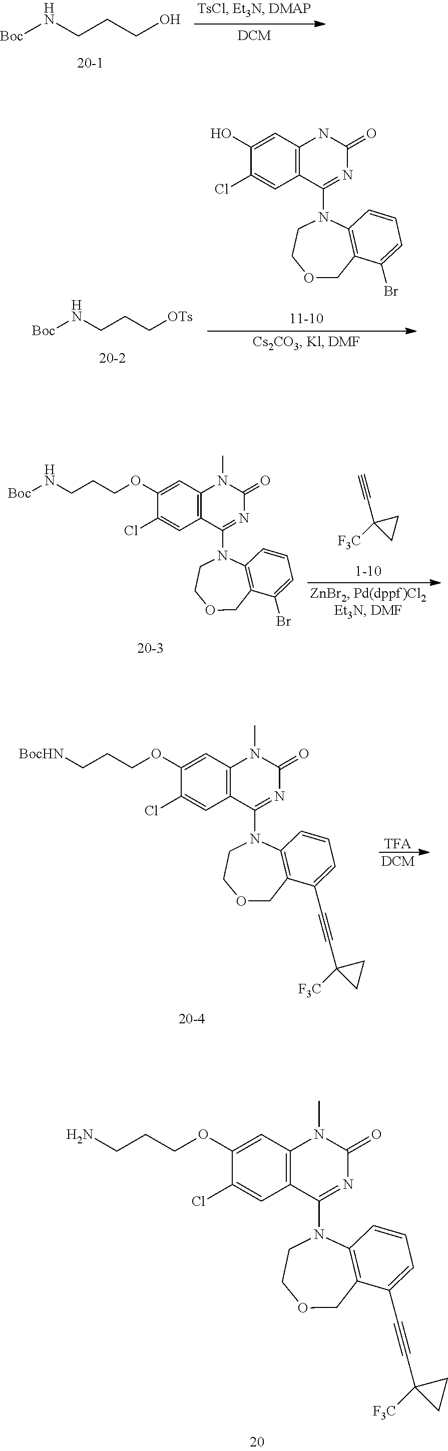

To a solution of 20-1 (5.0 g, 28.53 mmol) in DCM (50 mL) was added TEA (3.97 mL, 28.53 mmol), DMAP (0.35 g, 2.85 mmol) and TsCl (5.98 g, 31.39 mmol). The reaction mixture was stirred at 25° C. for 18 h. The mixture was poured into water (200 mL) and extracted with DCM (50 mL×3). The combined organic layers were dried over Na₂SO₄, filtered, and concentrated under reduced pressure. The resulting residue was purified by flash column chromatography on silica gel to give 20-2. MS (ESI) m/z (M+H−100)⁺=229.9.

To a solution of 11-10 (200 mg, 0.46 mmol) in DMF (10 mL) were added 20-2 (754 mg, 2.29 mmol), Cs₂CO₃ (1.49 g, 4.58 mmol) and potassium iodide (38.0 mg, 0.23 mmol) at 25° C. under N₂. The reaction mixture was stirred for 16 hour at 100° C. under N₂. The mixture was poured into water (100 mL) and extracted with EtOAc (50 mL×3). The combined organic layers were dried over Na₂SO₄, filtered, and concentrated under reduced pressure. The resulting residue was purified by flash column chromatography on silica gel to give 20-3. MS (ESI) m/z (M+H)⁺=593.0.

To a mixture of 20-3 (200 mg, 0.34 mmol) in TEA (2 mL) and DMF (2 mL) were added 1-10 (226 mg, 0.40 mmol), ZnBr₂ (379 mg, 1.68 mmol) and Pd(dppf)Cl₂ (24.6 mg, 0.034 mmol) at 25° C. The mixture was stirred at 100° C. for 0.5 h under N₂. The mixture was poured into water (100 mL) and extracted with DCM (50 mL×3). The combined organic layers were dried over Na₂SO₄, filtered, and concentrated under reduced pressure. The resulting residue was purified by flash column chromatography on silica gel to give 20-4. MS (ESI) m/z (M+H)⁺=647.2.

To a mixture of 20-4 (170 mg, 0.26 mmol) in DCM (5 mL) was added TFA (0.5 mL). The mixture was stirred at room temperature for 2 h under N₂. The mixture was poured into water (100 mL) and extracted with DCM (50 mL×3). The combined organic layers were dried over Na₂SO₄, filtered, and concentrated under reduced pressure. The resulting residue was purified by prep-HPLC separation to give example 20. MS (ESI) m/z (M+H)⁺=547.1. ¹H NMR (400 MHz, DMSO) δ 7.45 (d, J=7.2 Hz, 1H), 7.23 (t, J=7.9 Hz, 1H), 7.00 (d, J=7.6 Hz, 1H), 6.91 (s, 1H), 6.57 (s, 1H), 5.25-4.70 (m, 2H), 4.35-4.19 (m, 2H), 4.00-3.84 (m, 2H), 3.55 (s, 3H), 2.83-2.77 (m, 2H), 2.63-2.58 (m, 2H), 1.98-1.90 (m, 2H), 1.51-1.49 (m, 4H).

### Example 21: (S)-6-chloro-1-methyl-7-((tetrahydrofuran-3-yl)oxy)-4-(6-((1-(trifluoromethyl)cyclopropyl)ethynyl)-2,3-dihydrobenzo[e][1,4]oxazepin-1(5H)-yl)quinazolin-2(1H)-one

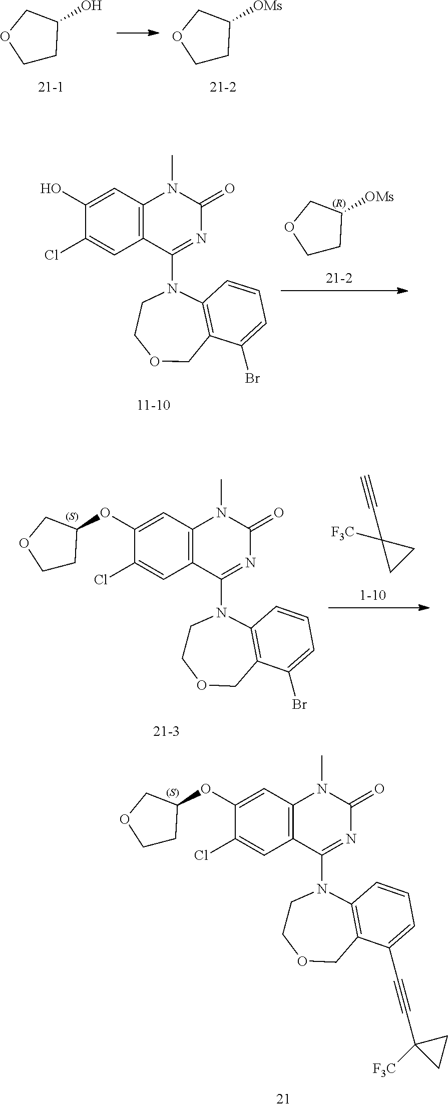

To a solution of 21-1 (3.65 mL, 45.4 mmol) in THF (100 mL) was added TEA (8.20 mL, 59.0 mmol) at 0° C. was added MsCl (6.76 g, 59.0 mmol). After stirring at 25° C. for 18 h under N₂, the mixture was quenched with H₂O (70 mL) and extracted with DCM (3×70 mL). The combined organic layers were dried over Na₂SO₄, filtered, and concentrated under reduced pressure to give compound 21-2 (5.5 g, 69.3%), which was used for the next step without further purification.

To a mixture of 11-10 (70 mg, 0.16 mmol) in DMF (10 mL) was added 21-2 (399 mg, 2.40 mmol), KI (13.3 mg, 0.080 mmol) and Cs₂CO₃ (522 mg, 1.60 mmol). After stirring at 100° C. for 3 h under N₂, the mixture was poured into water (20 mL) and extracted with EtOAc (3×20 mL). The combined organic layers were washed with brine (3×50 mL), dried over Na₂SO₄, filtered, and concentrated under reduced pressure. The resulting residue was purified by silica gel column chromatography to give 21-3 (60 mg, 73.9%). LCMS: MS (ESI) m/z (M+H)⁺=505.8.

To a mixture of 21-3 (60 mg, 0.12 mmol) in TEA (1 mL) and DMF (1 mL) were added 1-10 (119 mg, 0.14 mmol), ZnBr₂ (133 mg, 0.59 mmol) and Pd(dppf)Cl₂ (8.7 mg, 0.012 mmol) at 25° C. After stirring at 100° C. for 0.5 h in a sealed tube, the mixture was poured into water (10 mL) and extracted with EtOAc (3×10 mL). The combined organic layers were washed with brine (3×50 mL), dried over Na₂SO₄, filtered, and concentrated under reduced pressure. The resulting residue was purified by prep-HPLC to give example 21 (17.0 mg, 8.5%). LCMS: MS (ESI) m/z (M+H)⁺=560.2. ¹H NMR (400 MHz, DMSO) δ 7.46 (d, J=7.8 Hz, 1H), 7.24 (t, J=7.8 Hz, 1H), 7.02 (d, J=7.9 Hz, 1H), 6.86 (s, 1H), 6.57 (s, 1H), 5.41-5.36 (m, 1H), 5.25-4.80 (m, 2H), 4.00-3.70 (m, 6H), 3.55 (s, 3H), 3.41-3.36 (m, 2H), 2.35-2.25 (m, 1H), 2.05-1.95 (m, 1H), 1.57-1.43 (m, 4H).

### Example 22: (R)-6-chloro-1-methyl-7-((tetrahydrofuran-3-yl)oxy)-4-(6-((1-(trifluoromethyl)cyclopropyl)ethynyl)-2,3-dihydrobenzo[e][1,4]oxazepin-1(5H)-yl)quinazolin-2(1H)-one

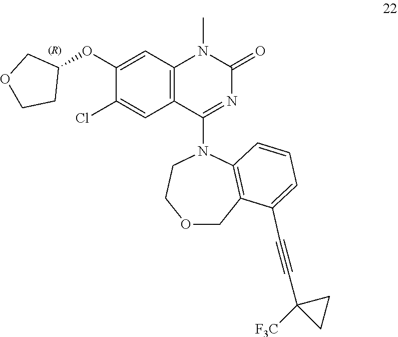

The preparation of example 22 referred to the similar procedure as example 21 with the reactant (R)-tetrahydrofuran-3-ol replaced by (S)-tetrahydrofuran-3-ol. LCMS: MS (ESI) m/z (M+H)⁺=560.2. ¹H NMR (400 MHz, DMSO) δ 7.46 (d, J=7.7 Hz, 1H), 7.24 (t, J=7.9 Hz, 1H), 7.02 (d, J=7.9 Hz, 1H), 6.86 (s, 1H), 6.57 (s, 1H), 5.41-5.35 (m, 1H), 5.25-4.75 (m, 2H), 3.98-3.71 (m, 6H), 3.55 (s, 3H), 3.31-3.25 (m, 2H), 2.33-2.26 (m, 1H), 2.05-1.93 (m, 1H), 1.51-1.45 (m, 4H).

### Example 23: (S)-1-methyl-2-oxo-7-((tetrahydrofuran-3-yl)oxy)-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

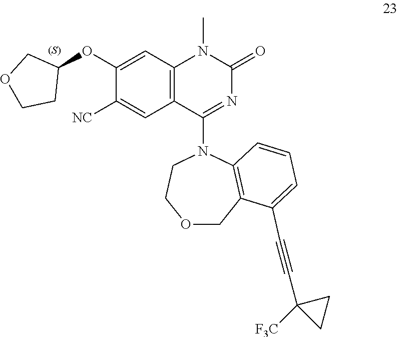

The preparation of example 23 referred to the similar procedure as example 12 with the reactant example 11 replaced by example 21. LCMS: MS (ESI) m/z (M+H)⁺=551.2. ¹H NMR (400 MHz, DMSO) δ 7.48 (dd, J=7.8, 1.0 Hz, 1H), 7.24 (t, J=7.9 Hz, 1H), 7.04 (dd, J=8.0, 1.0 Hz, 1H), 6.87 (s, 1H), 6.86 (s, 1H), 5.48-5.42 (m, 1H), 5.20-4.80 (m, 2H), 3.99-3.71 (m, 6H), 3.56 (s, 3H), 3.38-3.32 (m, 2H), 2.37-2.28 (m, 1H), 2.05-1.96 (m, 1H), 1.52-1.44 (m, 4H).

### Example 24: (R)-1-methyl-2-oxo-7-((tetrahydrofuran-3-yl)oxy)-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

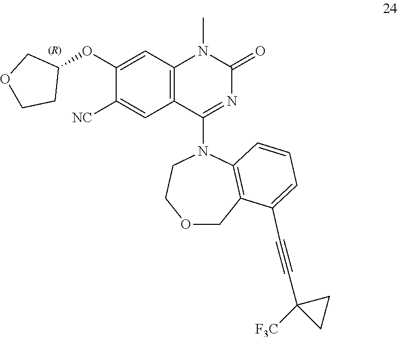

The preparation of example 24 referred to the similar procedure as example 12 with the reactant example 11 replaced by example 22. LCMS: MS (ESI) m/z (M+H)⁺=551.5. ¹H NMR (400 MHz, DMSO) δ 7.48 (d, J=7.6 Hz, 1H), 7.24 (t, J=7.9 Hz, 1H), 7.04 (d, J=7.8 Hz, 1H), 6.86 (d, J=2.1 Hz, 2H), 5.50-5.37 (m, 1H), 5.25-4.80 (m, 2H), 4.04-3.69 (m, 6H), 3.56 (s, 3H), 3.31-3.21 (m, 2H), 2.39-2.28 (m, 1H), 2.06-1.96 (m, 1H), 1.51-1.46 (m, 4H).

### Example 25: 6-chloro-1-methyl-7-((tetrahydro-2H-pyran-4-yl)oxy)-4-(6-((1-(trifluoromethyl)cyclopropyl)ethynyl)-2,3-dihydrobenzo[e][1,4]oxazepin-1(5H)-yl)quinazolin-2(1H)-one

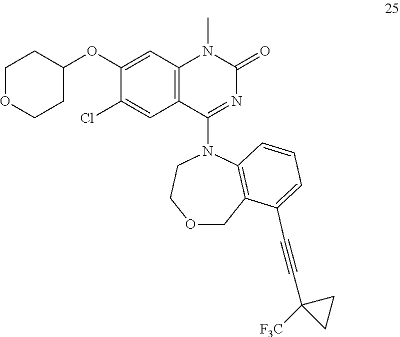 [SMILES: Cn1c(=O)nc(N2CCOCc3c(C#CC4(C(F)(F)F)CC4)cccc32)c2cc(Cl)c(OC3CCOCC3)cc21]

The preparation of example 25 referred to the similar procedure as example 11 with the reactant 3-bromotetrahydrofuran replaced by 4-bromotetrahydropyran. LCMS: MS (ESI) m/z (M+H)⁺=574.2. ¹H NMR δ 7.46 (d, J=7.6 Hz, 1H), 7.25 (t, J=7.6 Hz, 1H), 7.02 (d, J=8.0 Hz, 1H), 6.96 (s, 1H), 6.58 (s, 1H), 5.20-4.80 (m, 3H), 4.05-3.75 (m, 4H), 3.60-3.50 (m, 5H), 3.43-3.38 (m, 2H), 2.08-1.95 (m, 2H), 1.69-1.58 (m, 2H), 1.54-1.44 (m, 4H).

### Example 26: 1-methyl-2-oxo-7-((tetrahydro-2H-pyran-4-yl)oxy)-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

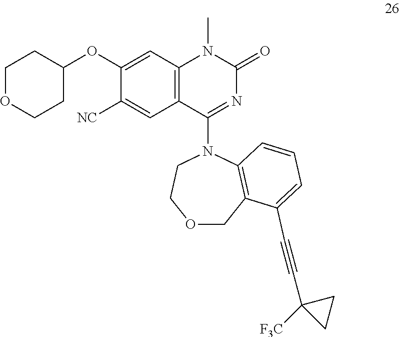 [SMILES: Cn1c(=O)nc(N2CCOCc3c(C#CC4(C(F)(F)F)CC4)cccc32)c2cc(C#N)c(OC3CCOCC3)cc21]

The preparation of example 26 referred to the similar procedure as example 12 with the reactant example 11 replaced by example 25. LCMS: MS (ESI) m/z (M+H)⁺=565.3. ¹H NMR (400 MHz, DMSO) δ 7.51-7.45 (m, 1H), 7.25 (t, J=8.0 Hz, 1H), 7.05 (d, J=7.2 Hz, 1H), 6.97 (s, 1H), 6.87 (s, 11H), 5.15-4.87 (m, 3H), 4.03-3.73 (m, 4H), 3.60-3.50 (m, 5H), 3.31-3.30 (m, 2H), 2.08-1.97 (m, 2H), 1.70-1.59 (m, 2H), 1.52-1.46 (m, 4H).

### Example 27: 6-chloro-7-(3-hydroxypropoxy)-1-methyl-4-(6-((1-(trifluoromethyl)cyclopropyl)ethynyl)-2,3-dihydrobenzo[e][1,4]oxazepin-1(5H)-yl)quinazolin-2(1H)-one

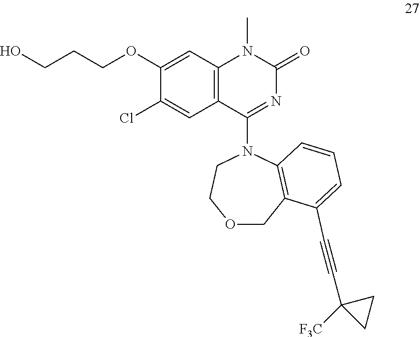 [SMILES: Cn1c(=O)nc(N2CCOCc3c(C#CC4(C(F)(F)F)CC4)cccc32)c2cc(Cl)c(OCCCO)cc21]

The preparation of example 27 referred to the similar procedure as example 17 with the reactant 1-bromo-2-methoxyethane replaced by 3-bromopropan-1-ol. LCMS: MS (ESI) m/z (M+H)⁺=548.0. ¹H NMR (400 MHz, DMSO) δ 7.45 (d, J=7.6 Hz, 1H), 7.23 (t, J=7.9 Hz, 1H), 7.00 (d, J=7.8 Hz, 1H), 6.92 (s, 1H), 6.56 (s, 1H), 5.13-4.85 (m, 2H), 4.63-4.55 (m, 1H), 4.27 (t, J=6.0 Hz, 2H), 3.99-3.83 (m, 2H), 3.59-3.52 (m, 5H), 3.35-3.31 (m, 2H), 1.94-1.86 (m, 2H), 1.52-1.46 (m, 4H).

### Example 28: 7-(3-hydroxypropoxy)-1-methyl-2-oxo-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

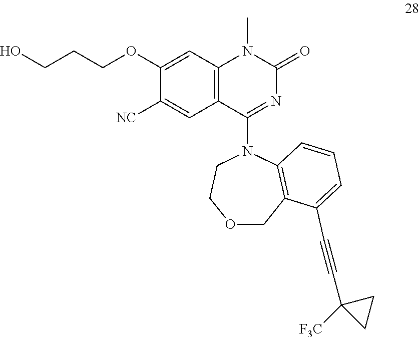 [SMILES: Cn1c(=O)nc(N2CCOCc3c(C#CC4(C(F)(F)F)CC4)cccc32)c2cc(C#N)c(OCCCO)cc21]

The preparation of example 28 referred to the similar procedure as example 12 with the reactant example 11 replaced by example 27. LCMS: MS (ESI) m/z (M+H)⁺=539.3. ¹H NMR (400 MHz, DMSO) δ 7.47 (d, J=7.6 Hz, 1H), 7.23 (t, J=8.0 Hz, 1H), 7.02 (d, J=8.0 Hz, 1H), 6.92 (s, 1H), 6.86 (s, 1H), 5.19-4.90 (m, 2H), 4.67-4.59 (m, 1H), 4.33 (t, J=6.0 Hz, 2H), 4.01-3.85 (m, 2H), 3.59-3.52 (m, 5H), 3.41-3.36 (m, 2H), 1.95-1.84 (m, 2H), 1.54-1.44 (m, 4H).

### Example 29: 6-chloro-7-(2-hydroxyethoxy)-1-methyl-4-(6-((1-methylcyclopropyl)ethynyl)-2,3-dihydrobenzo[e][1,4]oxazepin-1(5H)-yl)quinazolin-2(11H)-one

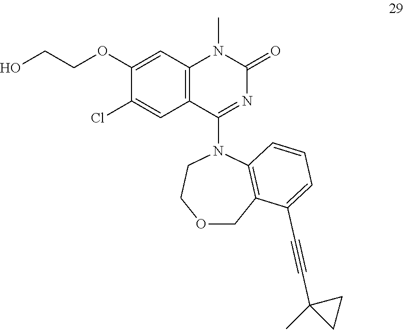 [SMILES: Cn1c(=O)nc(N2CCOCc3c(C#CC4(C)CC4)cccc32)c2cc(Cl)c(OCCO)cc21]

The preparation of example 29 referred to the similar procedure as example 13 with the intermediate 1-ethynyl-1-(trifluoromethyl)cyclopropane (1-10) replaced by 1-ethynyl-1-methylcyclopropane. LCMS: MS (ESI) m/z (M+H)⁺=480.0. ¹H NMR (400 MHz, DMSO) δ 7.36 (d, J=8.0 Hz, 1H), 7.21-7.15 (m, 1H), 6.96-6.87 (m, 2H), 6.56 (s, 1H), 5.40-4.70 (m, 3H), 4.33-4.17 (m, 2H), 3.97-3.82 (m, 2H), 3.80-3.72 (m, 2H), 3.54 (s, 3H), 3.49-3.45 (m, 2H), 1.37 (s, 3H), 1.05-0.97 (m, 2H), 0.84-0.76 (m, 2H).

### Example 30: 6-chloro-7-(2-hydroxyethoxy)-1-methyl-4-(6-((1-(trifluoromethyl)cyclopropyl)ethynyl)-2,3-dihydropyrido[2,3-e][1,4]oxazepin-1(5H)-yl)quinazolin-2(1H)-one

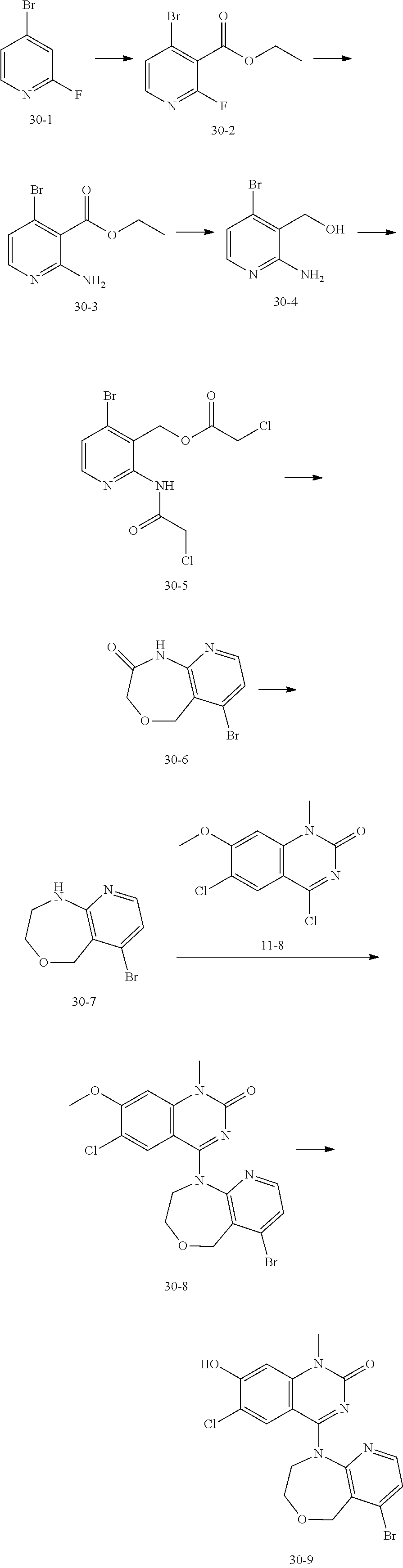

To a mixture of 30-1 (27 g, 153.4 mmol) in THF (200 mL) was added dropwise LDA (99.7 mL, 2M in THF) at −78° C. under N₂. The mixture was stirred at −78° C. for 30 min, and ethyl chloromethanoate (20 g, 184.3 mmol) was added to the above solution at −78° C. under N₂. After stirring at 25° C. for 16 h, the mixture was poured into water (100 mL) and extracted with EtOAc (3×100 mL). The combined organic layers were dried over Na₂SO₄, filtered, and concentrated under reduced pressure to give crude compound 30-2, which was directly used in the next step without further purification. LCMS: MS (ESI) m/z (M+H)⁺=247.9.

To a mixture of 30-2 (36 g, 145.1 mmol) in DMSO (80 mL) was added NH₄OH (40 mL, 145.1 mmol). After stirring at 100° C. for 3 h, the mixture was poured into water (100 mL) and extracted with EtOAc (3×100 mL). The mixture was concentrated under reduced pressure to give compound 30-3 (31 g, 87.2%). LCMS: MS (ESI) m/z (M+H)⁺=245.0.

To a mixture of 30-3 (31 g, 126.5 mmol) in THF (200 mL) at 0° C. was added LiAlH₄ (7.2 g, 189.7 mmol) under N₂. The mixture was stirred at 0° C. for 5 min and then warmed to 25° C. for 3 h. The mixture was quenched with sat. Na₂SO₄ solution and filtered. The filtrate was concentrated under reduced pressure. The resulting residue was purified by silica gel column chromatography to give 30-4 (7 g, 27.3%). LCMS: MS (ESI) m/z (M+H)⁺=202.9.

To a mixture of 30-4 (8.2 g, 40.4 mmol) in THF (150 mL) was added DIEA (33.4 mL) and chloroacetyl chloride (13.7 g, 121.2 mmol) at 0° C. After stirring at room temperature for 16 h, the mixture was quenched with aq. NaHCO₃ solution and extracted with DCM. The combined organic layers were dried over Na₂SO₄, filtered, and concentrated under reduced pressure. The resulting residue was purified by column chromatography on silica gel to give 30-5 (4.5 g, 31.3%). LCMS: MS (ESI) m/z (M+H)⁺=354.5.

A mixture of t-BuONa (4.2 g, 43.7 mmol) in t-BuOH (100 mL) was heated to 80° C. until all the solid was dissolved, 30-5 (7 g, 19.7 mmol) was added to the solution. After stirring at 80° C. for 2 h, the mixture was concentrated under reduced pressure. The resulting residue was purified by column chromatography on silica gel to give 30-6 (3.7 g, 77.4%). LCMS: MS (ESI) m/z (M+H)⁺=242.8.

To a mixture of 30-6 (2.9 g, 11.9 mmol) in THF (100 mL) was added BH₃THF (60 mL, 1 M in THF) at 0° C. After stirring at 80° C. for 2 h, MeOH (10 mL) and 1 N HCl (10 mL) were added to the mixture. The mixture was stirred at 80° C. for 2 h, and then quenched with aq. NaHCO₃ solution (30 mL) and extracted with DCM (50 mL×3). The combined organic layers were dried over Na₂SO₄, filtered, and concentrated under reduced pressure. The resulting residue was purified by column chromatography on silica gel to give 30-7 (1.1 g, 40.2%). LCMS: MS (ESI) m/z (M+H)⁺=229.1.

To a mixture of 30-7 (132 mg, 0.58 mmol) in THF (50 mL) was added LIHMDS (1.7 mL, 1 M in THF) at 20° C. under N₂. After stirring at 20° C. for 5 min under N₂, DIEA (0.64 mL, 3.86 mmol) and a solution of 11-8 (100 mg, 0.39 mmol) in THF (10 mL) were added to the above mixture. The mixture was stirred at room temperature for 2 h under N₂ and then quenched with aq. NH₄Cl solution (50 mL) and extracted with DCM (2×50 mL). The combined organic layers were dried over Na₂SO₄, filtered, and concentrated under reduced pressure. The resulting residue was purified by prep-TLC to give 30-8 (83 mg, 47.7%). LCMS: MS (ESI) m/z (M+H)⁺=450.7.

To a mixture of 30-8 (50 mg, 0.11 mmol) in DMF (3 mL) was added LiCl (46.9 mg, 1.11 mmol). After stirring at 140° C. for 18 h, the mixture was concentrated under reduced pressure. The resulting residue was diluted with DCM/MeOH (10/1, 15 mL) and filtered. The filtrate was concentrated under reduced pressure to give crude product 30-9, which was used for the next step without further purification. LCMS: MS (ESI) m/z (M+H)⁺=437.0.

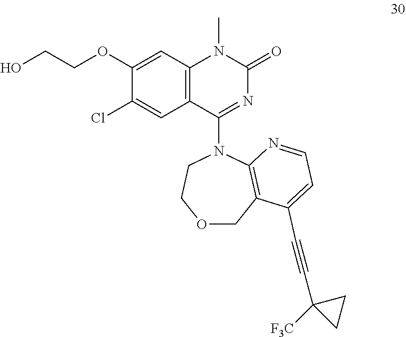 [SMILES: Cn1c(=O)nc(N2CCOCc3c(C#CC4(C(F)(F)F)CC4)ccnc32)c2cc(Cl)c(OCCO)cc21]

The preparation of example 30 referred to the similar procedure as example 13 with the intermediate 4-(6-bromo-2,3-dihydrobenzo[e][1,4]oxazepin-1(5H)-yl)-6-chloro-7-hydroxy-1-methylquinazolin-2(1H)-one (11-10) replaced by intermediate 4-(6-bromo-2,3-dihydropyrido[2,3-e][1,4]oxazepin-1(5H)-yl)-6-chloro-7-hydroxy-1-methylquinazolin-2(1H)-one (30-9). LCMS: MS (ESI) m/z (M+H)⁺=535.2. ¹H NMR ¹H NMR (400 MHz, DMSO) δ 8.09 (d, J=5.2 Hz, 1H), 7.32 (d, J=5.2 Hz, 1H), 6.97 (s, 1H), 6.65 (s, 1H), 5.18-4.78 (m, 3H), 4.27 (t, J=4.8 Hz, 2H), 3.98-3.89 (m, 2H), 3.79-3.74 (m, 2H), 3.59 (s, 3H), 3.33-3.31 (m, 2H), 1.57-1.52 (m, 4H).

### Example 31: 6-chloro-7-(2-hydroxyethoxy)-1-methyl-4-(6-((1-(trifluoromethyl)cyclopropyl)ethynyl)-2,3-dihydropyrido[3,4-e][1,4]oxazepin-1(5H)-yl)quinazolin-2(1H)-one

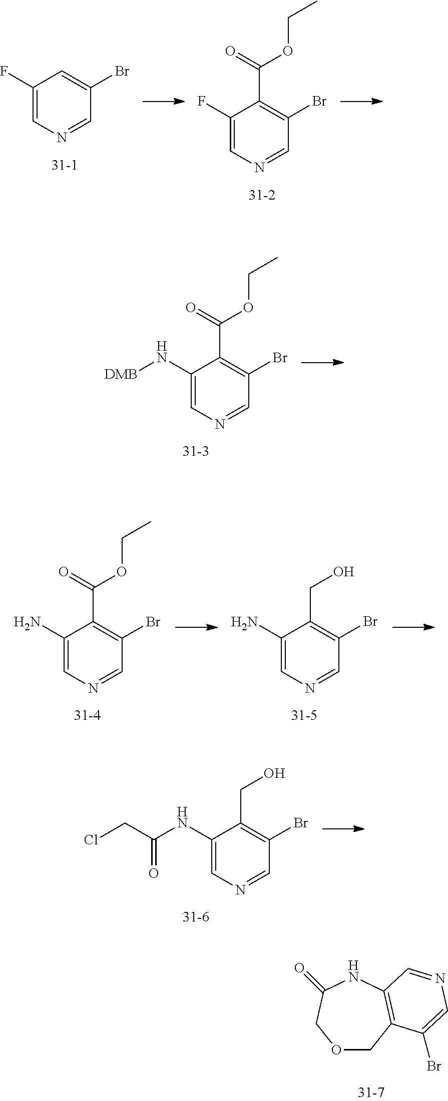

To a mixture of LDA (20.1 g, 187.5 mmol) in THE (100 mL) was added 31-1 (30.0 g, 170.5 mmol) at −78° C. under N₂. The reaction mixture was stirred for 1 h at −78° C. under N₂. Then ethyl chloromethanoate (22.2 g, 204.6 mmol) was added. After stirring for 1.5 h at 0° C. under N₂, the mixture was poured into water (100 mL) and extracted with EtOAc (3×10 mL). The combined organic layers were dried over Na₂SO₄, filtered, and concentrated under reduced pressure. The resulting residue was purified by silica gel column chromatography to give 31-2 (32.4 g, 76.6%). LCMS: MS (ESI) m/z (M+H)⁺=247.9.

To a mixture of 31-2 (32.4 g, 130.6 mmol) in DMF (400 mL) were added (2,4-dimethoxyphenyl)methanamine (32.8 g, 195.9 mmol) and K₂CO₃ (54.2 g, 391.9 mmol) at 20° C. After stirring at 50° C. for 12 h under N₂, the mixture was poured into water (100 mL) and extracted with EtOAc (3×100 mL). The combined organic layers were dried over Na₂SO₄, filtered, and concentrated under reduced pressure. The resulting residue was purified by flash column chromatography to give 31-3 (50.8 g, 98.4%).

To a mixture of 31-3 (4.0 g, 10.1 mmol) in DCM (30 mL) was added TFA (30 mL). After stirring at room temperature for 2 h under N₂. The mixture was concentrated under reduced pressure to give the crude, which was dissolved in DCM (100 mL) and adjusted to pH=8.0 with Sat. NaHCO₃(aq). The resulting mixture was extracted with DCM (3×50 mL). The combined organic layers were dried over Na₂SO₄, filtered, and concentrated under reduced pressure to give the crude, which was purified by silica gel column chromatography to give compound 31-4. LCMS: MS (EST) m/z (M+H)⁺=244.9.

To a mixture of 31-4 (8.76 g, 35.7 mmol) in THE (100 mL) was added LiAlH₄ (53.6 mL, 1 M in THF) at 0° C. under N₂. After stirring at room temperature for 18 h under N₂, the mixture was quenched with sat. Na₂SO₄ solution. The mixture was filtered and the filter cake was washed with THE (100 mL). The filtrate was concentrated under reduced pressure to give the crude, which was purified by flash column chromatography to give 31-5 (2.66 g, 36.7%). LCMS: MS (ESI) m/z (M+H)⁺=202.9.

To a mixture of 31-5 (2.66 g, 13.1 mmol) in THF (35 mL) was added DIEA (6.49 mL, 39.3 mmol), then chloroacetyl chloride (1.63 g, 14.4 mmol) was added to the mixture at 0° C. After stirring at room temperature for 16 h under N₂, the mixture was poured into water (50 mL) and extracted with EtOAc (3×50 mL). The combined organic layers were dried over Na₂SO₄, filtered, and concentrated under reduced pressure to give the crude, which was purified by flash column chromatography to give 31-6 (1.46 g, 39.9%). LCMS: MS (ESI) m/z (M+H)⁺=278.8.

To a mixture of t-BuONa (904 mg, 9.40 mmol) in t-BuOH (20 mL) was added 31-6 (1.46 g, 5.22 mmol). After stirring at 80° C. for 2 h under N₂, the mixture was concentrated under reduced pressure and quenched with H₂O (30 mL), and precipitation formed. The mixture was filtered and the filter cake was freeze dried to give compound 31-7, which was used for the next step without further purification. LCMS: MS (ESI) m/z (M+H)⁺=242.9.

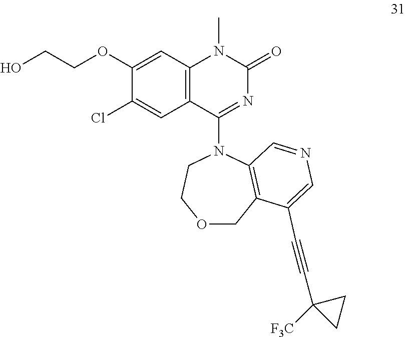 [SMILES: Cn1c(=O)nc(N2CCOCc3c(C#CC4(C(F)(F)F)CC4)cncc32)c2cc(Cl)c(OCCO)cc21]

The preparation of example 31 referred to the similar procedure as example 30 with the intermediate 6-bromo-1,5-dihydropyrido[2,3-e][1,4]oxazepin-2(3H)-one (30-6) replaced by intermediate 6-bromo-1,5-dihydropyrido[3,4-e][1,4]oxazepin-2(3H)-one (31-7). LCMS: MS (ESI) m/z (M+H)⁺=535.2. ¹H NMR (400 MHz, DMSO) δ 8.55 (s, 1H), 8.14 (s, 1H), 6.96 (s, 1H), 6.61 (s, 1H), 5.10-4.90 (m, 3H), 4.30-4.22 (m, 2H), 3.98-3.90 (m, 2H), 3.81-3.73 (m, 2H), 3.57 (s, 3H), 3.47-3.41 (m, 2H), 1.56-1.46 (m, 4H).

### Example 32: 7-(2-aminoethoxy)-6-chloro-1-methyl-4-(6-((1-(trifluoromethyl)cyclopropyl)ethynyl)-2,3-dihydrobenzo[e][1,4]oxazepin-1(5H)-yl)quinazolin-2(1H)-one

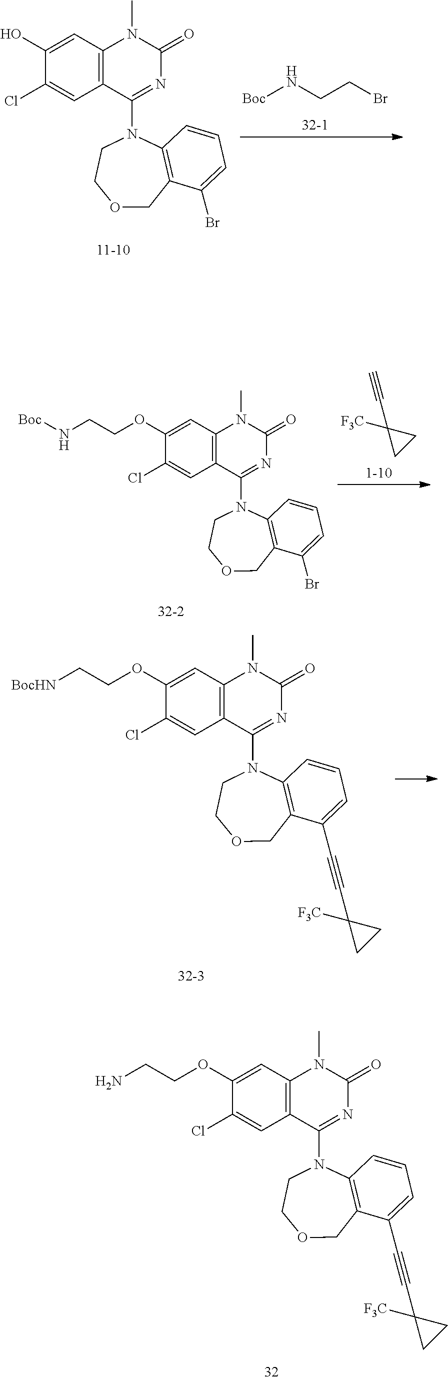

To a solution of 11-10 (150 mg, 0.34 mmol) in DMF (15 mL) were added 32-1 (384 mg, 1.72 mmol), KI (57 mg, 0.34 mmol) and Cs₂CO₃ (1.12 g, 3.43 mmol). After stirring at 100° C. for 3 hr, the reaction mixture was diluted with EtOAc (30 mL), and washed with brine (3×30 mL), dried over Na₂SO₄, filtered, and concentrated under reduced pressure. The resulting residue was purified by column chromatography on silica gel to give 32-2 (130 mg, 66.9%). LCMS: MS (ESI) m/z (M+H)⁺=579.0.

To a solution of 32-2 (130 mg, 0.22 mmol) in DMF (1 mL) were added TEA (0.62 mL, 4.48 mmol), ZnBr₂ (252 mg, 1.12 mmol), 1-10 (36 mg, 0.27 mmol) and Pd(dppf)Cl₂ (16.4 mg, 0.022 mmol). After stirring at 100° C. for 15 min in a sealed tube, the reaction was diluted with EtOAc (20 mL), and washed with brine (3×20 mL), dried over Na₂SO₄, filtered, and concentrated under reduced pressure. The resulting residue was purified by silica gel column chromatography to give 32-3 (100 mg, 70.5%). LCMS: MS (ESI) m/z (M+H)⁺=633.4.

To a solution of 32-3 (100 mg, 0.16 mmol) in DCM (3 mL) was added TFA (0.3 mL). The reaction mixture was stirred at room temperature for 1 hr. The reaction mixture was concentrated in vacuo and the resulting residue was purified by prep-HPLC to give example 32 (19.0 mg, 22.6%). LCMS: MS (ESI) m/z (M+H)⁺=533.4. ¹H NMR (400 MHz, CD₃OD) δ 7.48 (dd, J=7.8, 1.0 Hz, 1H), 7.26 (t, J=7.9 Hz, 1H), 7.03-6.97 (m, 1H), 6.60 (s, 1H), 6.51 (s, 1H), 5.30-4.95 (m, 2H), 4.00 (s, 2H), 3.78 (t, J=5.6 Hz, 2H), 3.63 (s, 3H), 3.45 (t, J=5.6 Hz, 2H), 3.36-3.32 (m, 2H), 1.50-1.37 (m, 4H).

### Example 33: 7-(2-aminoethoxy)-1-methyl-2-oxo-4-(6-((1-(trifluoromethyl)cyclopropyl)ethynyl)-2,3-dihydrobenzo[e][1,4]oxazepin-1(5H)-yl)-1,2-dihydroquinazoline-6-carbonitrile

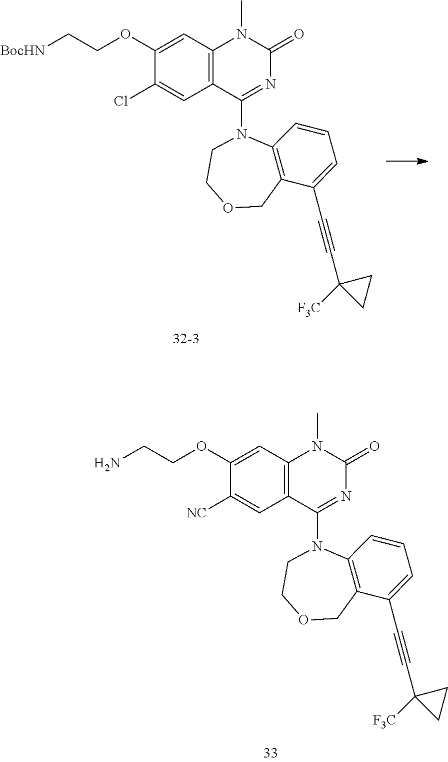 [SMILES: Cn1c(=O)nc(N2CCOCc3c(C#CC4(C(F)(F)F)CC4)cccc32)c2cc(Cl)c(OCCNC(=O)OC(C)(C)C)cc21 ; Cn1c(=O)nc(N2CCOCc3c(C#CC4(C(F)(F)F)CC4)cccc32)c2cc(C#N)c(OCCN)cc21]

To a mixture of 32-3 (40 mg, 0.063 mmol) in DMA (2 mL) was added Zn(CN)₂ (37 mg, 0.32 mmol), Zn (4.1 mg, 0.063 mmol), Zn(OAc)₂ (11.6 mg, 0.063 mmol), DPPF (17.8 mg, 0.032 mmol) and Pd₂(dba)₃CHCl₃ (32.6 mg, 0.032 mmol). The mixture was stirred at 160° C. for 1.5 h under microwave condition. Then TFA (0.2 mL) was added to the mixture, and the mixture was stirred at room temperature for another 1 h. The mixture was concentrated under reduced pressure. The residue was diluted with DCM (20 mL) and filtered through a pad of celite. The filtrate was concentrated under reduced pressure. The resulting residue was purified by prep-HPLC to give example 33 (1.8 mg, 4.8%). LCMS: MS (ESI) m/z (M+H)⁺=524.3. ¹H NMR (400 MHz, d6-DMSO) δ 7.45 (d, J=7.7 Hz, 1H), 7.24 (t, J=7.9 Hz, 1H), 7.01 (d, J=7.9 Hz, 1H), 6.68 (s, 1H), 6.36 (s, 1H), 5.16-4.91 (m, 2H), 3.99-3.81 (m, 2H), 3.71-3.50 (m, 4H), 3.47 (s, 3H), 3.42-3.30 (m, 2H), 1.52-1.46 (m, 4H).

### Example 34: 6-chloro-1-methyl-7-(pyrrolidin-3-yloxy)-4-(6-((1-(trifluoromethyl)cyclopropyl)ethynyl)-2,3-dihydrobenzo[e][1,4]oxazepin-1(5H)-yl)quinazolin-2(1H)-one

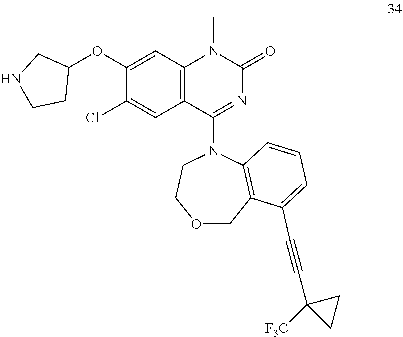 [SMILES: Cn1c(=O)nc(N2CCOCc3c(C#CC4(C(F)(F)F)CC4)cccc32)c2cc(Cl)c(OC3CCNC3)cc21]

The preparation of example 34 referred to the similar procedure as example 32 with the intermediate tert-butyl (2-bromoethyl)carbamate (32-1) replaced by intermediate tert-butyl 3-bromopyrrolidine-1-carboxylate. LCMS: MS (ESI) m/z (M+H)⁺=559.2. ¹H NMR (400 MHz, DMSO) δ 7.46 (d, J=7.7 Hz, 1H), 7.24 (t, J=7.8 Hz, 1H), 7.02 (d, J=7.9 Hz, 1H), 6.87 (s, 1H), 6.57 (s, 1H), 5.35-5.25 (m, 1H), 5.15-4.70 (m, 2H), 4.02-3.82 (m, 2H), 3.55 (s, 3H), 3.49-3.25 (m, 3H), 3.07-2.97 (m, 3H), 2.22-2.07 (m, 1H), 2.00-1.82 (m, 1H), 1.54-1.44 (m, 4H).

### Example 35: 6-chloro-1-methyl-7-((1-methylpyrrolidin-3-yl)oxy)-4-(6-((1-(trifluoromethyl)cyclopropyl)ethynyl)-2,3-dihydrobenzo[e][1,4]oxazepin-1(5H)-yl)quinazolin-2(1H)-one

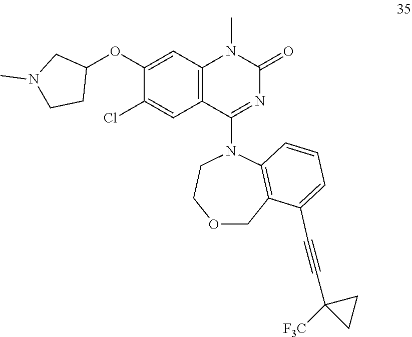 [SMILES: CN1CCC(Oc2cc3c(cc2Cl)c(N2CCOCc4c(C#CC5(C(F)(F)F)CC5)cccc42)nc(=O)n3C)C1]

The preparation of example 35 referred to the similar procedure as example 21 with the intermediate (R)-tetrahydrofuran-3-ol (21-1) replaced by intermediate 1-methylpyrrolidin-3-ol. LCMS: MS (ESI) m/z (M+H)⁺=573.2. ¹H NMR (400 MHz, DMSO) δ 7.46 (d, J=7.5 Hz, 1H), 7.24 (t, J=7.9 Hz, 1H), 7.02 (d, J=7.9 Hz, 1H), 6.78 (s, 1H), 6.56 (s, 1H), 5.30-4.80 (m, 3H), 4.05-3.75 (m, 2H), 3.54 (s, 3H), 2.87-2.77 (m, 1H), 2.72-2.63 (m, 2H), 2.48-2.30 (m, 4H), 2.26 (s, 3H), 1.85-1.73 (m, 1H), 1.53-1.45 (m, 4H).

### Example 36 & 37: 6-chloro-4-(2,3-dihydrobenzo[e][1,4]oxazepin-1(5H)-yl)-7-(2-hydroxyethoxy)-1-methylquinazolin-2(1H)-one and 6-chloro-4-(6-cyclopropyl-2,3-dihydrobenzo[e][1,4]oxazepin-1(5H)-yl)-7-(2-hydroxyethoxy)-1-methylquinazolin-2(1H)-one

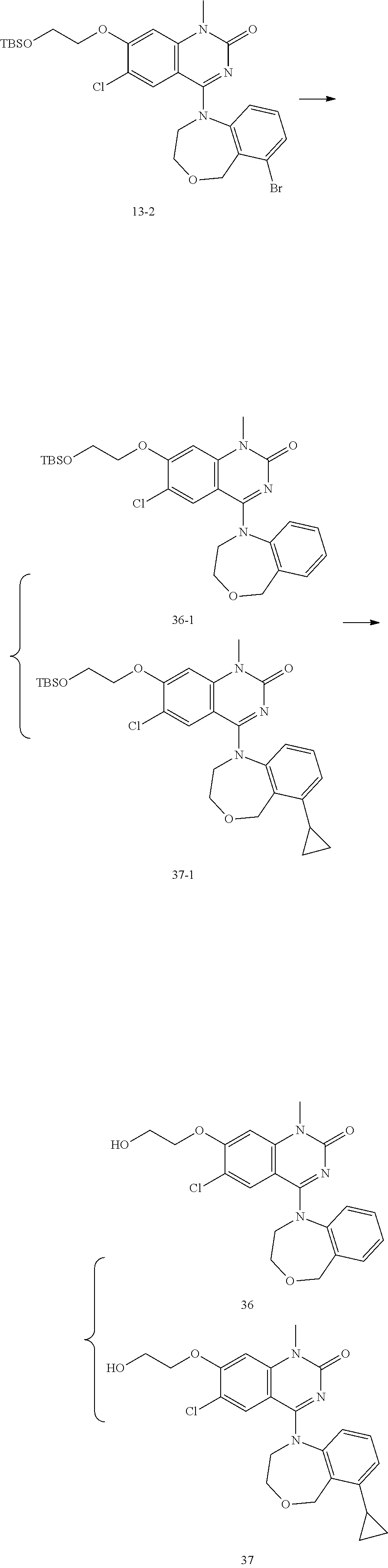 [SMILES: Cn1c(=O)nc(N2CCOCc3c(Br)cccc32)c2cc(Cl)c(OCCO[Si](C)(C)C(C)(C)C)cc21 ; Cn1c(=O)nc(N2CCOCc3ccccc32)c2cc(Cl)c(OCCO[Si](C)(C)C(C)(C)C)cc21 ; Cn1c(=O)nc(N2CCOCc3c(C4CC4)cccc32)c2cc(Cl)c(OCCO[Si](C)(C)C(C)(C)C)cc21 ; Cn1c(=O)nc(N2CCOCc3ccccc32)c2cc(Cl)c(OCCO)cc21 ; Cn1c(=O)nc(N2CCOCc3c(C4CC4)cccc32)c2cc(Cl)c(OCCO)cc21]

A mixture of 13-2 (100 mg, 0.17 mmol), cyclopropylboronic acid (20 mg, 0.23 mmol), K₂CO₃ (69.65 mg, 0.504 mmol) and Pd(dppf)Cl₂ (20 mg, 0.027 mmol) in dioxane (2 mL) and H₂O (0.2 mL) was stirred at 100° C. for 2 h under N₂. The mixture was added with water (10 mL) and extracted with EtOAc (10 mL×3). The combined organic layers were dried over Na₂SO₄, filtered, and concentrated under reduced pressure to give a mixture of compound 36-1 and compound 37-1. LCMS: MS (ESI) m/z (M+H)⁺=516.2, 556.2.

To a mixture of 36-1 and 37-1 (120 mg) in THE (5 mL) was added TBAF solution (0.4 mL, 1M in THF) dropwise. After stirring at room temperature for 1 h, the mixture was added with water (10 mL) and extracted with EtOAc (3×10 mL). The combined organic layers were washed with brine (10 mL), dried over Na₂SO₄, filtered, and concentrated under reduced pressure. The resulting residue was purified by Prep-HPLC to give example 36 (12.4 mg) and example 37 (24.8 mg).

Example 36: LCMS: MS (ESI) m/z (M+H)⁺=402.0. ¹H NMR (400 MHz, DMSO) δ 7.58 (d, J=6.5 Hz, 1H), 7.34 (t, J=7.4 Hz, 1H), 7.24 (t, J=7.6 Hz, 1H), 6.95 (d, J=7.6 Hz, 1H), 6.92 (s, 1H), 6.52 (s, 1H), 5.35-4.55 (m, 3H), 4.24 (t, J=4.7 Hz, 2H), 4.05-3.82 (m, 2H), 3.76 (t, J=4.7 Hz, 2H), 3.55 (s, 3H), 3.41-3.35 (m, 2H).

Example 37: LCMS: MS (ESI) m/z (M+H)⁺=442.3. ¹H NMR (400 MHz, DMSO) δ 7.18-7.07 (m, 2H), 6.90 (s, 1H), 6.80 (d, J=7.3 Hz, 1H), 6.51 (s, 1H), 5.55-4.65 (m, 3H), 4.24 (t, J=4.7 Hz, 2H), 4.05-3.85 (m, 2H), 3.75 (q, J=4.9 Hz, 2H), 3.54 (s, 3H), 3.37-3.33 (m, 2H), 2.24-2.14 (m, 1H), 1.01 (d, J=7.2 Hz, 2H), 0.80-0.60 (m, 2H).

### Example 38: 6-chloro-7-(2-hydroxyethoxy)-1-methyl-4-(6-methyl-2,3-dihydrobenzo[e][1,4]oxazepin-1(5H)-yl)quinazolin-2(11H)-one

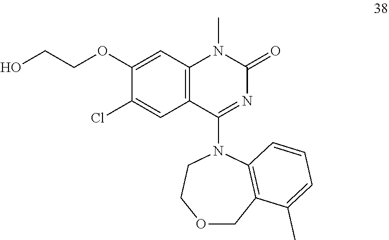 [SMILES: Cc1cccc2c1COCCN2c1nc(=O)n(C)c2cc(OCCO)c(Cl)cc12]

The preparation of example 38 referred to the similar procedure as example 37 with the intermediate cyclopropylboronic acid replaced by intermediate methylboronic acid. LCMS: MS (ESI) m/z (M+H)⁺=416.1. ¹H NMR (400 MHz, DMSO) δ 7.23 (d, J=7.4 Hz, 1H), 7.13 (t, J=7.6 Hz, 1H), 6.90 (s, 1H), 6.79 (d, J=7.7 Hz, 1H), 6.57 (s, 1H), 5.10-4.70 (m, 3H), 4.30-4.19 (m, 2H), 3.98-3.82 (m, 2H), 3.79-3.71 (m, 2H), 3.54 (s, 3H), 3.49-3.44 (m, 2H), 2.46 (s, 3H).

### Example 39: 6-chloro-4-(6-ethynyl-2,3-dihydrobenzo[e][1,4]oxazepin-1(5H)-yl)-7-(2-hydroxyethoxy)-1-methylquinazolin-2(1H)-one

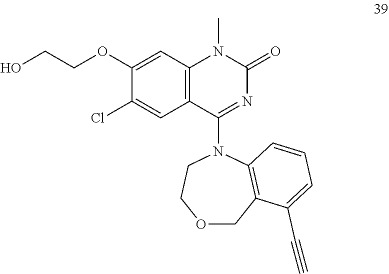 [SMILES: C#Cc1cccc2c1COCCN2c1nc(=O)n(C)c2cc(OCCO)c(Cl)cc12]

The preparation of example 39 referred to the similar procedure as example 13 with the intermediate 1-ethynyl-1-(trifluoromethyl)cyclopropane (1-10) replaced by intermediate ethynyltrimethylsilane. LCMS: MS (ESI) m/z (M+H)⁺=426.0. ¹H NMR (400 MHz, DMSO) δ 7.50 (d, J=7.7 Hz, 1H), 7.24 (t, J=7.9 Hz, 1H), 7.01 (d, J=7.7 Hz, 1H), 6.93 (s, 1H), 6.56 (s, 1H), 5.26-4.80 (m, 3H), 4.60 (s, 1H), 4.24 (t, J=4.7 Hz, 2H), 4.01-3.82 (m, 2H), 3.78-3.72 (m, 2H), 3.55 (s, 3H), 3.30-3.28 (m, 2H).

### Example 40: 6-chloro-7-(2-hydroxyethoxy)-1-methyl-4-(6-(prop-1-yn-1-yl)-2,3-dihydrobenzo[e][1,4]oxazepin-1(5H)-yl)quinazolin-2(1H)-one

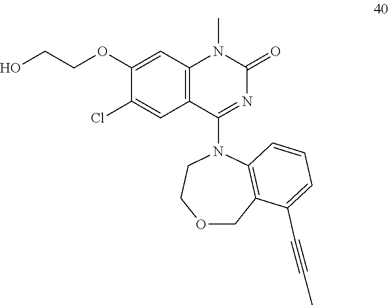 [SMILES: CC#Cc1cccc2c1COCCN2c1nc(=O)n(C)c2cc(OCCO)c(Cl)cc12]

The preparation of example 40 referred to the similar procedure as example 13 with the intermediate 1-ethynyl-1-(trifluoromethyl)cyclopropane (1-10) replaced by intermediate prop-1-yne. LCMS: MS (ESI) m/z (M+H)⁺=440.1. ¹H NMR (400 MHz, DMSO) δ 7.41 (d, J=7.8 Hz, 1H), 7.19 (t, J=7.9 Hz, 1H), 6.95-6.89 (m, 2H), 6.54 (s, 1H), 5.35-4.65 (m, 3H), 4.24 (t, J=4.8 Hz, 2H), 4.02-3.84 (m, 2H), 3.79-3.70 (m, 2H), 3.55 (s, 3H), 3.31-3.26 (m, 2H), 2.14 (s, 3H).

### Example 41: 6-chloro-4-(6-(cyclopropylethynyl)-2,3-dihydrobenzo[e][1,4]oxazepin-1(5H)-yl)-7-(2-hydroxyethoxy)-1-methylquinazolin-2(1H)-one

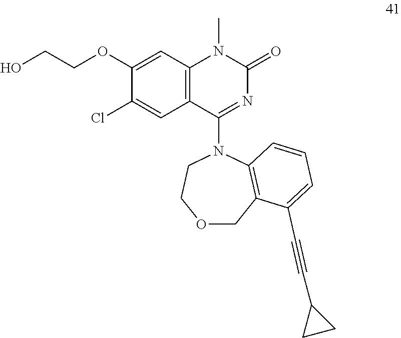 [SMILES: Cn1c(=O)nc(N2CCOCc3c(C#CC4CC4)cccc32)c2cc(Cl)c(OCCO)cc21]

The preparation of example 41 referred to the similar procedure as example 13 with the intermediate 1-ethynyl-1-(trifluoromethyl)cyclopropane (1-10) replaced by intermediate ethynylcyclopropane. LCMS: MS (ESI) m/z (M+H)⁺=466.0. ¹H NMR (400 MHz, DMSO) δ 7.38 (d, J=7.4 Hz, 1H), 7.18 (t, J=7.9 Hz, 1H), 6.94-6.88 (m, 2H), 6.55 (s, 1H), 5.20-4.70 (m, 3H), 4.24 (t, J=4.6 Hz, 2H), 4.04-3.82 (m, 2H), 3.79-3.72 (m, 2H), 3.54 (s, 3H), 3.31-3.24 (m, 2H), 1.71-1.58 (m, 1H), 1.01-0.90 (m, 2H), 0.85-0.77 (m, 2H).

### Example 42: 6-chloro-7-(2-hydroxyethoxy)-4-(6-methoxy-2,3-dihydrobenzo[e][1,4]oxazepin-1(5H)-yl)-1-methylquinazolin-2(1H)-one

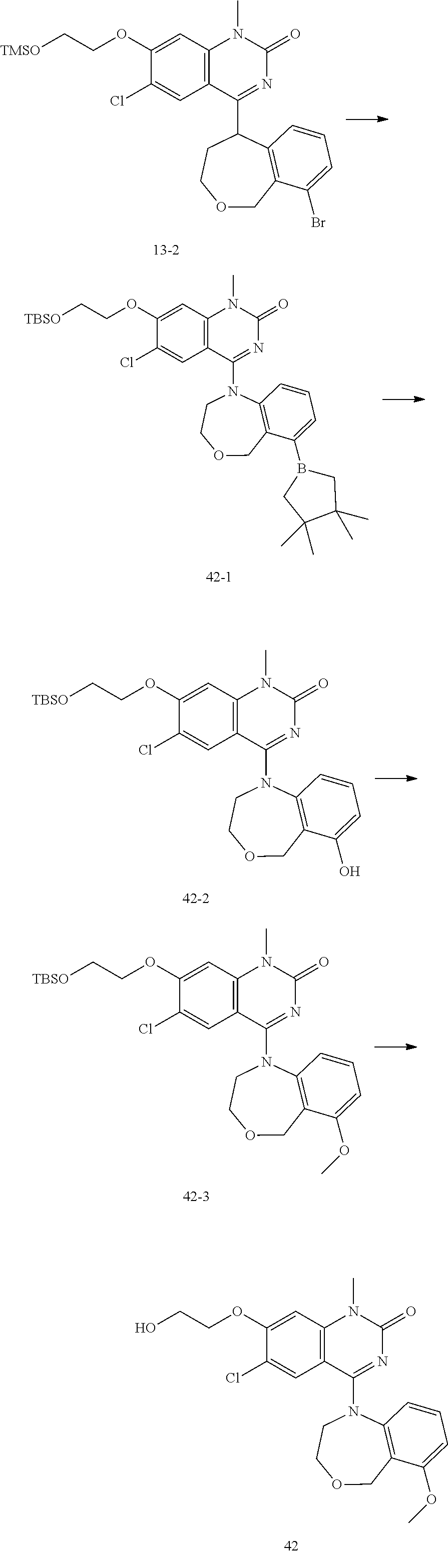

To a mixture of 13-2 (2.0 g, 3.36 mmol) in dioxane (20 mL) was added B₂(pin)₂ (1.7 g, 6.72 mmol), Pd(dppf)Cl₂ (0.20 g, 0.336 mmol) and KOAc (1.0 g, 10.1 mmol). After stirring at 100° C. for 3 h under N₂, the mixture was diluted with ethyl acetate (90 mL). The organic layer was washed with H₂O (50 mL×2), dried over Na₂SO₄, and concentrated to give compound 42-1 (2.0 g). LCMS: MS (ESI) m/z (M+H)⁺=642.0.

To a mixture of 42-1 (2.0 g, 3.12 mmol) in ACN (20 mL) was added urea hydrogen peroxide (0.60 g, 6.23 mmol). After stirring at room temperature overnight, the mixture was diluted with H₂O (100 mL) and extracted with ethyl acetate (50 mL×3). The combined organic layers were washed with water (70 mL×2), dried over Na₂SO₄, filtered, and concentrated under reduced pressure. The resulting residue was purified by column chromatography on silica gel to give 42-2 (1.1 g, 66.4%). LCMS: MS (ESI) m/z (M+H)⁺=532.2.

To a mixture of 42-2 (30 mg, 0.056 mmol) in DMF (2 mL) was added NaH (3 mg, 60% in mineral oil) at 0° C. under N₂. The mixture was stirred for 15 min, and then iodomethane (41 mg, 0.29 mmol) was added. The reaction mixture was stirred at room temperature overnight under N₂. The mixture was diluted with ethyl acetate (15 mL) and washed with H₂O (10 mL×2), dried over Na₂SO₄, filtered, and concentrated under reduced pressure. The resulting residue was purified by column chromatography on silica gel to give compound 42-3 (25 mg, 81.2%). LCMS: MS (ESI) m/z (M+H)⁺=546.0.

To a mixture of 42-3 (45 mg, 0.082 mmol) in THF (2 mL) was added TBAF (0.17 mL, 1M in THF). After stirring at room temperature for 1 h. The mixture was diluted with ethyl acetate (20 mL) and washed with H₂O (3×20 mL), dried over Na₂SO₄, filtered, and concentrated under reduced pressure. The resulting residue was purified by prep-HPLC to give example 42 (14.1 mg, 39.6%). LCMS: MS (ESI) m/z (M+H)⁺=432.1. ¹H NMR (400 MHz, DMSO) δ 7.20 (t, J=8.1 Hz, 1H), 7.04 (d, J=8.2 Hz, 1H), 6.92 (s, 1H), 6.68 (s, 1H), 6.56 (d, J=8.0 Hz, 1H), 4.96 (t, J=5.1 Hz, 1H), 4.27-4.21 (m, 2H), 3.90-3.85 (m, 4H), 3.78-3.73 (m, 2H), 3.56 (s, 3H), 3.37-3.34 (m, 2H), 3.31 (s, 3H).

### Example 43: 6-chloro-4-(6-cyclopropoxy-2,3-dihydrobenzo[e][1,4]oxazepin-1(5H)-yl)-7-(2-hydroxyethoxy)-1-methylquinazolin-2(1H)-one

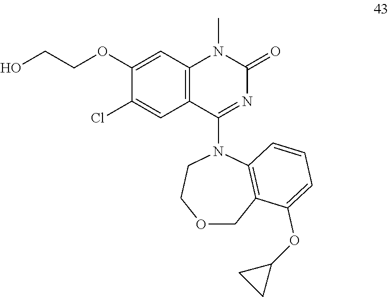 [SMILES: Cn1c(=O)nc(N2CCOCc3c(OC4CC4)cccc32)c2cc(Cl)c(OCCO)cc21]

The preparation of example 43 referred to the similar procedure as example 42 with the intermediate iodomethane replaced by intermediate cyclopropylbromide. LCMS: MS (ESI) m/z (M+H)⁺=458.0. ¹H NMR (400 MHz, DMSO) δ 7.32 (d, J=8.0 Hz, 1H), 7.21 (t, J=8.0 Hz, 1H), 6.90 (s, 1H), 6.60 (s, 1H), 6.56 (d, J=8.0 Hz, 1H), 5.20-4.75 (m, 2H), 3.31-4.18 (m, 2H), 4.02-3.93 (m, 1H), 3.88-3.82 (m, 2H), 3.81-3.70 (m, 2H), 3.53 (s, 3H), 3.48-3.37 (m, 2H), 3.88-3.80 (m, 2H), 3.78-3.70 (m, 2H).

### Example 44: 4-(6-cyclopropyl-2,3-dihydrobenzo[e][1,4]oxazepin-1(5H)-yl)-7-(2-hydroxyethoxy)-1-methyl-2-oxo-1,2-dihydroquinazoline-6-carbonitrile

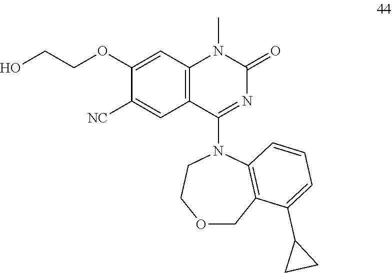 [SMILES: Cn1c(=O)nc(N2CCOCc3c(C4CC4)cccc32)c2cc(C#N)c(OCCO)cc21]

The preparation of example 44 referred to the similar procedure as example 14 with the intermediate 7-(2-((tert-butyldimethylsilyl)oxy)ethoxy)-6-chloro-1-methyl-4-(6-((1-(trifluoromethyl)cyclopropyl)ethynyl)-2,3-dihydrobenzo[e][1,4]oxazepin-1(5H)-yl)quinazolin-2(1H)-one (13-3) replaced by intermediate 7-(2-((tert-butyldimethylsilyl)oxy)ethoxy)-6-chloro-4-(6-cyclopropyl-2,3-dihydrobenzo[e][1,4]oxazepin-1(5H)-yl)-1-methylquinazolin-2(1H)-one (37-1). LCMS: MS (ESI) m/z (M+H)⁺=433.2. ¹H NMR (400 MHz, MeOD) δ 7.28-7.12 (m, 2H), 6.96 (s, 1H), 6.86-6.79 (m, 2H), 5.70-5.38 (m, 2H), 4.38-4.26 (m, 2H), 4.09-3.96 (m, 2H), 3.65 (s, 3H), 3.45-3.26 (m, 2H), 2.24-2.08 (m, 11H), 1.11-0.99 (m, 2H), 0.90-0.62 (m, 2H).

### Example 45: 4-(6-ethyl-2,3-dihydrobenzo[e][1,4]oxazepin-1(5H)-yl)-7-(2-hydroxyethoxy)-1-methyl-2-oxo-1,2-dihydroquinazoline-6-carbonitrile

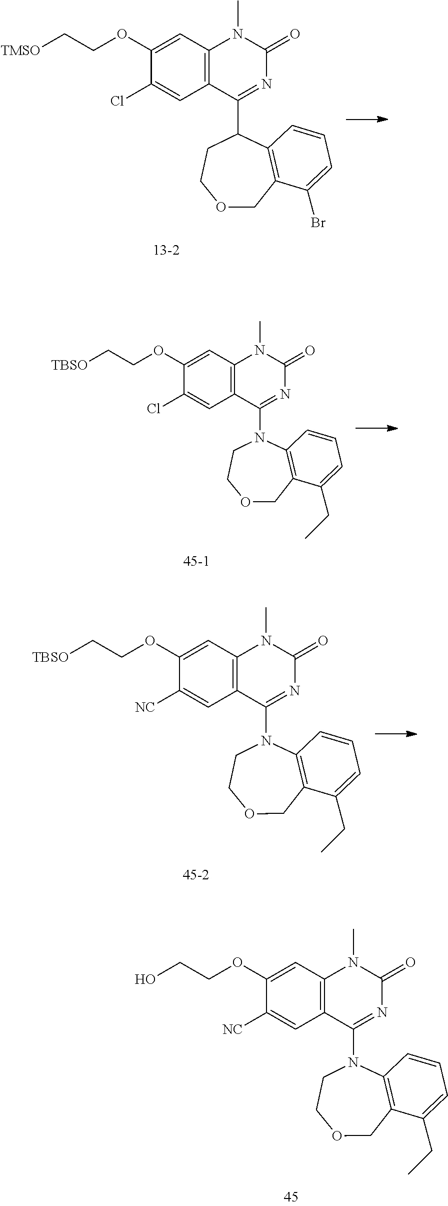

To a solution of 13-2 (150 mg, 0.25 mmol) in dioxane (10 mL) was added ethylboronic acid (37 mg, 0.50 mmol), K₂CO₃ (105 mg, 0.76 mmol) and PdCl₂(dppf) (18 mg, 0.025 mmol). After stirring at 100° C. overnight under N₂, the mixture was poured into water (30 mL) and extracted with EA (3×10 mL). The combined organic layers were dried over Na₂SO₄, filtered, and concentrated under reduced pressure. The resulting residue was purified by silica gel column chromatography to give 45-1 (130 mg, 94.8%). LCMS: MS (ESI) m/z (M+H)⁺=544.3.

To a solution of 45-1 (130 mg, 0.24 mmol) in DMA (3 mL) were added Zn(CN)₂ (56 mg, 0.48 mmol), Zn (16 mg, 0.24 mmol), Zn(OAc)₂ (44 mg, 0.24 mmol), Pd₂(dba)₃ (22 mg, 0.024 mmol) and DPPF (41 mg, 0.072 mmol). The mixture was stirred at 150° C. for 1 h in a sealed tube under microwave, and then the mixture was filtered through a pad of celite. The filtrate was poured into water (30 mL) and extracted with EA (3×10 mL). The combined organic layers were dried over Na₂SO₄, filtered, and concentrated under reduced pressure. The resulting residue was purified by silica gel column chromatography to give 45-2 (120 mg, 93.9%). LCMS: MS (ESI) m/z (M+H)⁺=535.3.

To a mixture of 45-2 (120 mg, 0.22 mmol) in THF (10 mL) was added TBAF (0.23 mL, 1 M in THF). After stirring at room temperature for 2 h, the mixture was quenched with water (30 mL) and extracted with ethyl acetate (10 mL×3). The combined organic layers were washed with water (10 mL), filtered, and concentrated under reduced pressure. The resulting residue was purified by prep-HPLC to give example 45 (11.5 mg). LCMS: MS (ESI) m/z (M+H)⁺=421.0. ¹H NMR (400 MHz, DMSO) δ 7.27 (d, J=7.2 Hz, 1H), 7.18 (t, J=7.6 Hz, 1H), 6.91 (s, 1H), 6.84 (d, J=7.6 Hz, 1H), 6.69 (s, 1H), 5.05-4.70 (m, 3H), 4.33-4.25 (m, 2H), 4.00-3.82 (m, 2H), 3.77-3.72 (m, 2H), 3.53 (s, 3H), 3.34-3.33 (m, 2H), 2.82 (d, J=7.6 Hz, 2H), 1.18 (t, J=7.6 Hz, 3H).

### Example 46: 7-(2-hydroxyethoxy)-1-methyl-2-oxo-4-(6-(pyrrolidin-1-yl)-2,3-dihydrobenzo[e][1,4]oxazepin-1(5H)-yl)-1,2-dihydroquinazoline-6-carbonitrile

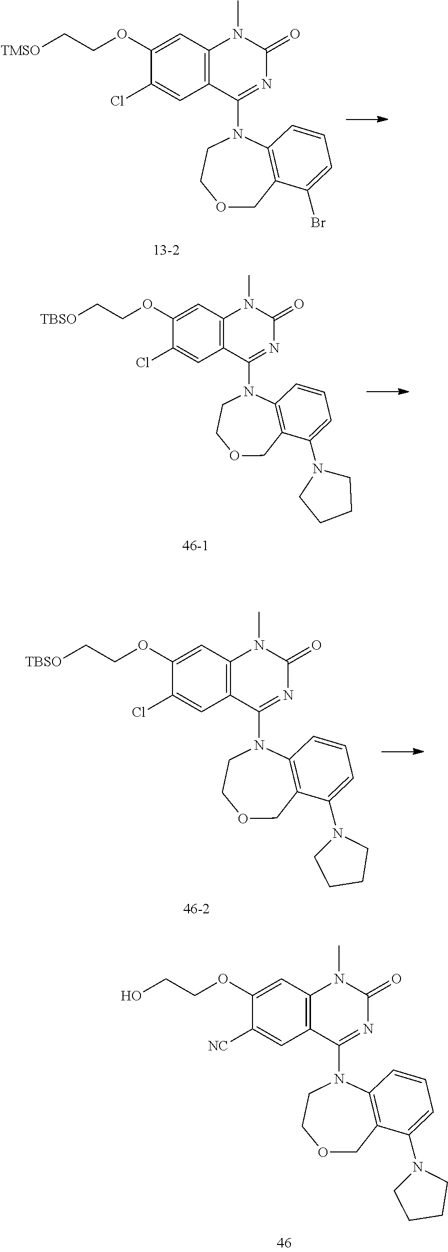

A mixture of 13-2 (110 mg, 0.19 mmol), pyrrolidine (0.04 mL, 0.46 mmol), Xphos (18 mg, 0.037 mmol), Cs₂CO₃ (181 mg, 0.56 mmol) and Pd₂(dba)₃ (17 mg, 0.018 mmol) in toluene (2 mL) was stirred at 80° C. for 2 h in a sealed tube under N₂. The mixture was added with water (10 mL) and extracted with EtOAc (3×10 mL). The combined organic layers were washed with brine (2×20 mL), dried over Na₂SO₄, filtered, and concentrated under reduced pressure. The resulting residue was purified by column chromatography on silica gel to give compound 46-1 (100 mg, 92.4%). LCMS: MS (ESI) m/z (M+H)⁺=585.4.

To a solution of 46-1 (100 mg, 0.17 mmol) in DMA (3 mL) was added Zn (11 mg, 0.17 mmol), Zn(CN)₂ (40 mg, 0.34 mmol), Zn(OAc)₂ (31 mg, 0.17 mmol), dppf (19 mg, 0.034 mmol) and Pd₂(dba)₃-CHCl₃ (18 mg, 0.017 mmol). After stirring under microwave at 150° C. for 1 h in a sealed tube, the mixture was diluted with EtOAc (10 mL) and filtered. The filtrate was washed with water (2×10 mL) and brine (10 mL), dried over Na₂SO₄, filtered, and concentrated under reduced pressure. The resulting residue was purified by column chromatography on silica gel to give compound 46-2 (50 mg, 50.8%). LCMS: MS (ESI) m/z (M+H)⁺=576.1.

To a mixture of 46-2 (50 mg, 0.087 mmol) in THE (2 mL) was added TBAF (0.17 mL, 1M in THF). After stirring at room temperature for 1 h, the mixture was diluted with EtOAc (15 mL). The mixture was washed with H₂O (3×20 mL), dried over Na₂SO₄, filtered, and concentrated under reduced pressure. The resulting residue was purified by prep-HPLC to give example 46 (13.6 mg, 33.8%). LCMS: MS (ESI) m/z (M+H)⁺=462.1. ¹H NMR (400 MHz, DMSO) δ 7.00 (t, J=7.9 Hz, 1H), 6.93-6.89 (m, 2H), 6.88-6.85 (m, 1H), 6.40 (d, J=7.7 Hz, 1H), 5.35-4.91 (m, 3H), 4.33-4.27 (m, 2H), 4.04-3.80 (m, 2H), 3.79-3.70 (m, 2H), 3.56 (s, 3H), 3.51-3.33 (m, 2H), 2.99-2.73 (m, 4H), 1.97-1.84 (m, 4H).

### Example 47: 4-(6-(cyclopropylethynyl)-2,3-dihydrobenzo[e][1,4]oxazepin-1(5H)-yl)-7-(2-hydroxyethoxy)-1-methyl-2-oxo-1,2-dihydroquinazoline-6-carbonitrile

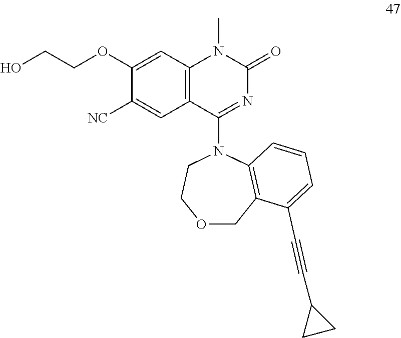 [SMILES: Cn1c(=O)nc(N2CCOCc3c(C#CC4CC4)cccc32)c2cc(C#N)c(OCCO)cc21]

The preparation of example 47 referred to the similar procedure as example 13 and example 14 with the intermediate 1-ethynyl-1-(trifluoromethyl)cyclopropane (1-10) replaced by intermediate ethynylcyclopropane. LCMS: MS (ESI) m/z (M+H)⁺=457.2. ¹H NMR (400 MHz, DMSO) δ 7.40 (d, J=8.0 Hz, 1H), 7.18 (t, J=8.0 Hz, 1H), 6.97-6.90 (m, 2H), 6.81 (s, 1H), 5.19-4.79 (m, 3H), 4.30 (t, J=4.6 Hz, 2H), 4.01-3.80 (m, 2H), 3.80-3.72 (m, 2H), 3.54 (s, 3H), 3.41-3.27 (m, 2H), 1.72-1.57 (m, 1H), 1.02-0.90 (m, 2H), 0.87-0.77 (m, 2H).

### Example A: DGKA Inhibition ADP-Glo Assay

The DGKA inhibition reactions were performed using ADP-Glo assay. The reactions were carried out in 50 mM HEPES, 100 mM NaCl, 10 mM MgCl₂, 1 mM CaCl₂, 0.005% TritonX-100 and 1 mM DTT as working solution (pH=7.5). 30 nL DMSO solution of each test compound (Top concentration 0.4 M with 10 point, 4-fold dilution series for each compound) were transferred to 384 well plate by Echo and 5 μL DGKA (SignalChem, D21-10BG) enzyme working solution at 2× final reaction concentration was added to each well. After incubated at 25° C. for 15 minutes, 5 μL substrate working solution contain 40 μM ATP (Promega, V915B) and 200 M DLG (SignalChem, D430-59) were added to initiate reaction. After enzymatic reaction at 25° C. for 60 min, ADP Glo assay reagents (Promega, V9102) were added and luminescence was recorded using an EnVision following the instruction of manual. The percent inhibition was calculated from luminescence by no enzyme control reactions for 100% inhibition and DMSO only reactions for 0% inhibition. The IC₅₀ value was calculated via a regression analysis of the inhibition rate.

The data for Example A is shown in Table 3.

TABLE 3

| Example | DGKA IC₅₀ |
|---|---|
| 1 | A |
| 2 | A |
| 3 | B |
| 4 | B |
| 5 | A |
| 6 | A |
| 7 | A |
| 8 | A |
| 9 | A |
| 10 | B |
| 11 | A |
| 12 | A |
| 13 | A |
| 14 | A |
| 15 | A |
| 16 | A |
| 17 | A |
| 18 | A |
| 19 | B |
| 20 | B |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | B |
| 35 | B |
| 36 | B |
| 37 | B |
| 38 | B |
| 39 | A |
| 40 | A |
| 41 | A |
| 42 | B |
| 43 | A |
| 44 | C |
| 45 | B |
| 46 | A |
| 47 | A |

IC₅₀ (nM): 0 < A ≤ 1; 1 < B ≤ 10; 10 < C ≤ 100

### Example B: IL-2 Release Detection Assay

Isolate Human Pan T cells from PBMC according to T cell isolation kit (Stemcell, 17951). The suspend T cell in RPMI 1640 medium containing 10% FBS, 1% PS and 55 M β-Mer at a density of 3*10\hat{ }6 cells/well/3 ml are seeded into 6 well plate, recovered overnight at 37° C.&55% CO₂. Harvest overnight recovered T cells are suspended in fresh RPMI1640 medium containing 10% FBS, 1% PS and 55 μM f-Mer, seeded 1*10\hat{ }5/well/200 μl cells into the anti-CD₃ pre-coated 96 well plate. Add dilutions of compounds into the cells and incubate at 37° C.&5% CO₂ for 24 hours. IL-2 in cell supernatant was detected by ELISA Kit (R&D, DY202). Fit the compound EC₅₀ in non-linear regression equation: Y=Bottom+(Top−Bottom)/(1+10\hat{ }((Log EC50−X)*HillSlope)), X=compound concentration, Y=Activation %. Top and Bottom: Plateaus in same units as Y. Log EC₅₀: same log units as X. Activation fold: Top activation/DMSO control. Activation % at 100 nM: Activation of non-linear regression equation curve at the concentration of 100 nM/DMSO control*100%.

The data for Example B is shown in Table 4.

TABLE 4
IL-2 release detection assay

| | | | Activation % |
|---|---|---|---|
| Example | EC₅₀ (nM) | Activation fold | at 100 nM |
| 3 | 422 | 6.3 | 247 |
| 4 | 479 | 10.0 | 313 |
| 5 | 444 | 7.7 | 395 |
| 9 | 1371 | 13.2 | 180 |
| 10 | 2606 | 5.8 | 95.5 |
| 11 | 335 | 8.6 | 324 |
| 12 | 1077 | 14.2 | 311 |
| 13 | 131 | 22.5 | 1232 |
| 14 | 265 | 15.3 | 639 |
| 17 | 1005 | 10.2 | 202 |
| 19 | 2188 | 25.2 | 234 |
| 20 | 62.8 | 1.7 | 173 |
| 25 | 243 | 5.4 | 275 |
| 32 | 702 | 33.9 | 540 |
| 33 | 714 | 38.4 | 631 |
| 41 | 608 | 26.3 | 510 |

### Example C: Pharmacokinetic Profile Evaluation

Species and strain: Male CD-1 mice of SPF. Source: Sino-British SIPPR/BK Lab Animal Ltd, Shanghai. 3 mice were orally gavage administrated with given compounds (Formulation: 5% DMSO+10% Solutol+85% Saline). The blood samples were taken via cephalic vein at timepoints 0.25 h, 0.5 h, 1 h, 2 h, 4 h, 6 h, 8 h and 24 h after oral gavage administration, 30 μL/time point. Blood samples were placed in tubes containing K2-EDTA and stored on ice until centrifuged. The blood samples were centrifuged at 6800 g for 6 minutes at 2-8° C. within 1 h after collected and stored frozen at approximately −80° C. An aliquot of 10 μL plasma samples were protein precipitated with 200 μL MeOH in which contains 10 ng/mL Verapamil (IS). The mixture was vortexed for 1 min and centrifuged at 18000 g for 7 min. Transfer 180 μL supernatant to 96 well plates. An aliquot of 6 μL supernatant was injected for LC-MS/MS analysis by LC-MS/MS-04 (API4000) instrument. The analytical results were confirmed using quality control samples for intra-assay variation. The accuracy of >66.7% of the quality control samples should be between 80-120% of the known value(s). Standard set of parameters including Area Under the Curve (AUC(0-t) and AUC(0-∞)), elimination half-live (T½), maximum plasma concentration (Cmax), will be calculated using noncompartmental analysis modules in FDA certified pharmacokinetic program Phoenix WinNonlin 7.0 (Pharsight, USA).

Species and strain: Male SD rats of SPF. Source: Sino-British SIPPR/BK Lab Animal Ltd, Shanghai. 3 rats were orally gavage administrated with given compounds (Formulation: 5% DMSO+10% Solutol+85% Saline). The blood samples were taken via submandibular vein or other suitable vein at timepoints 0.25 h, 0.5 h, 1 h, 2 h, 4 h, 6 h, 8 h and 24 h after oral gavage administration, 0.15 mL/time point. Blood samples were placed in tubes containing K2-EDTA and stored on ice until centrifuged. The blood samples were centrifuged at 6800 g for 6 minutes at 2-8° C. within 1 h after collected and stored frozen at approximately −80° C. An aliquot of 30 μL plasma samples were protein precipitated with 300 μL MeOH in which contains 100 ng/mL Verapamil (IS). The mixture was vortexed for 1 min and centrifuged at 18000 g for 7 min. Transfer 300 μL supernatant to 96 well plates. An aliquot of 8 μL supernatant was injected for LC-MS/MS analysis by LC-MS/MS-12 (TQ5500) instrument. The analytical results were confirmed using quality control samples for intra-assay variation. The accuracy of >66.7% of the quality control samples should be between 80-120% of the known value(s). Standard set of parameters including Area Under the Curve (AUC₍₀₋ₜ₎ and AUC_{(0-∞)}), elimination half-live (T_{1/2}), maximum plasma concentration (Cₘₐₓ), will be calculated using noncompartmental analysis modules in FDA certified pharmacokinetic program Phoenix WinNonlin 7.0 (Pharsight, USA).

The data for Example C is shown in Table 5 and 6:

TABLE 5
Mouse PK profile after oral administration at 5 mg/kg

| | | Cmax | AUC_{0~t} | AUC_{0~∞} |
|---|---|---|---|---|
| Example | T_{1/2} (h) | (ng/mL) | (ng*h/mL) | (ng*h/mL) |
| 5 | 2.03 | 985 | 6655 | 7109 |
| 12 | 1.03 | 1691 | 7743 | 7869 |
| 13 | 6.13 | 2401 | 26211 | 28382 |
| 41 | 3.28 | 2228 | 15339 | 15459 |

TABLE 6
Rat PK profile after oral administration at 5 mg/kg

| | | Cmax | AUC_{0~t} | AUC_{0~∞} |
|---|---|---|---|---|
| Example | T_{1/2} (h) | (ng/mL) | (ng*h/mL) | (ng*h/mL) |
| 5 | 6.32 | 292 | 3466 | 3761 |
| 13 | NA | 455 | 7517 | NA |
| 41 | 8.09 | 724 | 10223 | 12031 |

Examples 13 and 41 showed longer T_{1/2}, much higher Cₘₐₓ and AUC compared with compound based on both mouse and rat PK evaluation results.

What is claimed is:

1. A compound of Formula (A), or a pharmaceutically acceptable salt thereof: 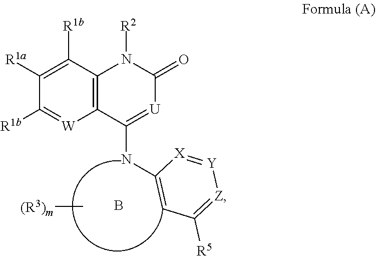
wherein:
R^{1a} is halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
W is N or CR^{1b};
each R^{1b} is independently hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
U is N or CR^{U};
R^{U} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Ring B is a heterocycloalkyl;
each R³ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R³ on the same atom are taken together to form an oxo;
or two R³ on the same or different carbons are taken together to form a cycloalkyl or heterocycloalkyl, each optionally substituted with one or more R;
m is 0-6;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
R⁵ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, 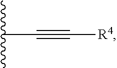
cycloalkyl, heterocycloalkyl, aryl, or heteroaryl; wherein each alkyl, alkenyl, cycloalkyl, heterocycloalkyl, aryl, and heteroaryl is optionally and independently substituted with one or more R;
R⁴ is hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{4a};
each R^{4a} is independently halogen, —CN, —NO₂, —OH, —OR^{a}, —OC(═O)R^{a}, —OC(O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or two R^{4a} on the same atom are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

2. The compound of claim 1, or a pharmaceutically acceptable salt thereof, wherein the compound is of Formula (Aa):

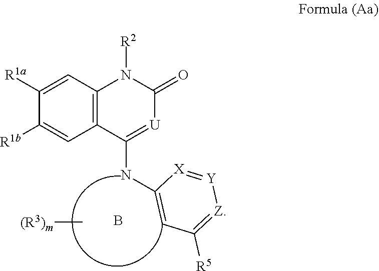

3. The compound of claim 1 or 2, or a pharmaceutically acceptable salt thereof, wherein R^{1a} is —OR^{a}.

4. The compound of claim 1 or 2, or a pharmaceutically acceptable salt thereof, wherein R^{1b} is halogen or —CN.

5. The compound of any one of claims 1-4, or a pharmaceutically acceptable salt thereof, wherein R² is hydrogen or C₁-C₆alkyl.

6. The compound of any one of claims 1-5, or a pharmaceutically acceptable salt thereof, wherein U is N.

7. The compound of any one of claims 1-6, or a pharmaceutically acceptable salt thereof, wherein Ring B is a 6- to 8-membered heterocycloalkyl.

8. The compound of any one of claims 1-7, or a pharmaceutically acceptable salt thereof, wherein 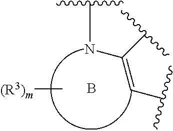
is

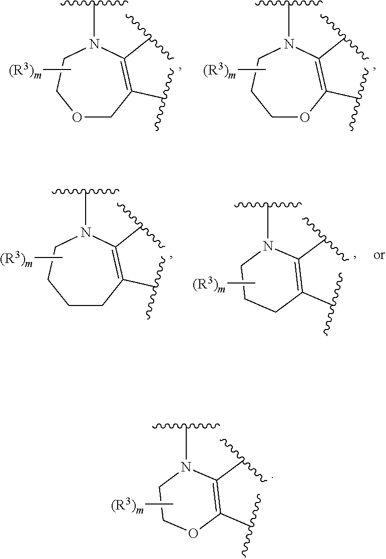

9. The compound of any one of claims 1-8, or a pharmaceutically acceptable salt thereof, wherein m is 0.

10. The compound of any one of claims 1-9, or a pharmaceutically acceptable salt thereof, wherein R^{X} is hydrogen.

11. The compound of any one of claims 1-10, or a pharmaceutically acceptable salt thereof, wherein R^{Y} is hydrogen.

12. The compound of any one of claims 1-11, or a pharmaceutically acceptable salt thereof, wherein R^{Z} is hydrogen.

13. The compound of any one of claims 1-12, or a pharmaceutically acceptable salt thereof, wherein R⁵ is

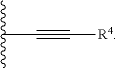

14. The compound of any one of claims 1-13, or a pharmaceutically acceptable salt thereof, wherein R⁴ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{4a}.

15. The compound of any one of claims 1-14, or a pharmaceutically acceptable salt thereof, wherein R⁴ is 3- to 6-membered cycloalkyl, 5- to 6-membered heterocycloalkyl, phenyl, or 5- to 6-membered heteroaryl; wherein the cycloalkyl, heterocycloalkyl, aryl, and heteroaryl is optionally substituted with one or more R^{4a}.

16. The compound of any one of claims 1-15, or a pharmaceutically acceptable salt thereof, wherein R⁴ is cyclopropyl, cyclobutyl, cyclopentyl, or cyclohexyl; each optionally substituted with one or more R^{4a}.

17. The compound of any one of claims 1-16, or a pharmaceutically acceptable salt thereof, wherein each R^{4a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, 3- to 6-membered cycloalkyl, or 5- to 6-membered heterocycloalkyl; wherein each alkyl, cycloalkyl, and heterocycloalkyl is optionally and independently substituted with one or more R.

18. The compound of claim 1, or a pharmaceutically acceptable salt thereof, wherein the compound is selected from a compound found in table 1 or table 2.

19. A pharmaceutical composition comprising the compound of any one of claims 1-18, or a pharmaceutically acceptable salt thereof, and a pharmaceutically acceptable excipient.

20. A method of inhibiting the activity of at least one of diacylglycerol kinase selected from diacylglycerol kinase alpha (DGKalpha) and diacylglycerol kinase zeta (DGKzeta), in a subject in need thereof, the method comprising administering to the subject the compound of any one of claims 1-18, or a pharmaceutically acceptable salt thereof.

21. A method of inhibiting the activity of diacylglycerol kinase alpha (DGKalpha), in a subject in need thereof, the method comprising administering to the subject the compound of any one of claims 1-18, or a pharmaceutically acceptable salt thereof.

22. A method of modulating the activity of at least one of diacylglycerol kinase selected from diacylglycerol kinase alpha (DGKalpha) and diacylglycerol kinase zeta (DGKzeta), in a subject in need thereof, the method comprising administering to the subject the compound of any one of claims 1-18, or a pharmaceutically acceptable salt thereof.

23. A method of modulating the activity of diacylglycerol kinase alpha (DGKalpha), in a subject in need thereof, the method comprising administering to the subject the compound of any one of claims 1-18, or a pharmaceutically acceptable salt thereof.

24. A method of treating a disease associated with aberrant diacylglycerol kinase signaling, in a subject in need thereof, the method comprising administering to the subject the compound of any one of claims 1-18, or a pharmaceutically acceptable salt thereof.

25. The method of claim 24, wherein the diacylglycerol kinase is diacylglycerol kinase alpha.

26. A method of treating a disease in a subject in need thereof, the method comprising administering to the subject the compound of any one of claims 1-18, or a pharmaceutically acceptable salt thereof.

27. The method of claim 25 or 26, wherein the disease is cancer or a viral infection.

28. The method of any one of claims 20-27, wherein the method further comprises administering an additional therapeutic agent.

29. The method of claim 28, wherein the additional therapeutic agent is an anti-cancer agent or an anti-viral agent.
